(19) United States
(12) Patent Application Publication
(10) Pub. No.: US 2026/0000698 A1
(43) Pub. Date: Jan. 1, 2026

(54) SARS-COV-2 NSP14 METHYLTRANSFERASE INHIBITORS

(71) Applicant: REGENTS OF THE UNIVERSITY OF MINNESOTA, MINNEAPOLIS, MN (US)

(72) Inventors: Liqiang CHEN, MINNEAPOLIS, MN (US); Robert GERAGHTY, MINNEAPOLIS, MN (US); Eunkyung JUNG, MINNEAPOLIS, MN (US)

(21) Appl. No.: 18/869,192

(22) PCT Filed: May 23, 2023
(86) PCT No.: PCT/US2023/023237

§ 371 Date: Nov. 25, 2024

Related U.S. Application Data
(60) Provisional application No. 63/344,943, filed on May 23, 2022.

(51) Int. Cl. A61K 31/7076; A61K 31/7064; A61P 31/14; C07D 487/04; C07H 19/167; C07H 19/23

(52) U.S. Cl. CPC A61K 31/7076; A61K 31/7064; A61P 31/14; C07D 487/04; C07H 19/167; C07H 19/23

(57) ABSTRACT

Disclosed herein are compounds of formula

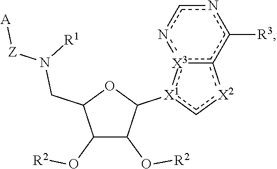

or pharmaceutically acceptable salts thereof, and methods of using the same for treating a viral infection or inhibiting replication of a virus.

### CROSS-REFERENCE TO RELATED APPLICATIONS

The present application claims priority to U.S. Provisional Patent Application No. 63/344,943, filed May 23, 2022, the entire contents of which are hereby incorporated by reference.

### STATEMENT REGARDING FEDERALLY SPONSORED RESEARCH

This invention was made with government support under AI166065 awarded by the National Institutes of Health. The government has certain rights in the invention.

### BACKGROUND OF THE INVENTION

Coronaviruses are enveloped, single-stranded positive-sense RNA viruses that belong to the family Coronaviridae. Severe acute respiratory syndrome coronavirus-2 (SARS-CoV-2) is a member of the genus Betacoronavirus along with the severe acute respiratory syndrome coronavirus (SARS-CoV) and the Middle East respiratory syndrome coronavirus (MERS-CoV).¹ As the causative agent of coronavirus disease 2019 (COVID-19), SARS-CoV-2 has posed a global health threat unseen in modern history, infecting more than 470 million and killing over 6 million people worldwide since the end of 2019.² While the availability of vaccines, antibodies, and the antiviral drug remdesivir in combination with new approvals of paxlovid and molnupiravir have offered hope to end this devastating pandemic, recent emergence and rapid spread of more infectious variants call for continued efforts to develop effective therapeutics to combat SARS-CoV-2 infection.

### BRIEF SUMMARY OF THE INVENTION

Disclosed herein are inhibitors of a viral methyltransferase, including SARS-CoV-2 Nsp14 methyltransferase, and methods of using the same. One aspect of the technology is directed to compounds of formula

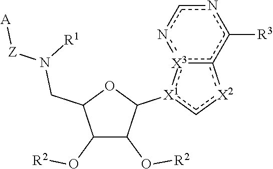

or a pharmaceutically acceptable salt or prodrug thereof,
wherein—
(i) X¹ is C, X² is C—R⁴, and X³ is N, or (ii) X¹ is N, X² is N, and X³ is C, or (iii) X¹ is C;
X² is S, X³ is C, or (iv) X¹ is N, X² is C—R⁴, and X³ is C;
R¹ is H or an alkyl;
each R² is independently selected from H, an alkyl, and a —C(O)-alkyl;
R³ is an amino or an amido;
R⁴ is H or a halo;
Q is O or —CH₂—.
Z is a sulfonyl, a carbonyl, or an alkyl; and
A is an aryl, a heteroaryl, or a biaryl, wherein the bicyclic aryl or bicyclic heteroaryl is optionally substituted in one or more positions with a halo, an alkyl optionally substituted at one or more position with a halo, an alkoxyl optionally substituted at one or more position with a halo, nitro, or cyano; and
   wherein when X¹ is N, X² is N, and X³ is C; R² is H; R³ is amino; and Q is O, then A is not a monocyclic aryl.

Another aspect of the technology is directed to pharmaceutical compositions comprising the compounds disclosed herein. In some embodiments, the pharmaceutical composition comprises an effective amount of the compound to treat a viral infection or inhibit replication of a virus. In some embodiments, the virus is SARS-CoV-2 or the viral infection is a SARS-CoV-2 infection.

Another aspect of the technology is directed to methods of treating a viral infection, such as a SARS-CoV-2 infection, by administering the compounds disclosed herein to a subject in need of a treatment for the viral infection. In some embodiments, the effective amount inhibits replication of the virus.

Another aspect of the invention is directed to inhibiting viral replication, such as SARS-CoV-2 replication, by contacting a cellular medium having a virus therein with the compounds disclosed herein. In some embodiments, the contacting the cellular medium with the compound occurs in vivo. In other embodiments, contacting the cellular medium with the compound occurs in vitro or ex vivo.

### DETAILED DESCRIPTION OF THE INVENTION

Disclosed herein are inhibitors of a viral methyltransferase, including SARS-CoV-2 Nsp14 methyltransferase, and methods of using the same. The disclosed compounds are bi-substrate inhibitors interacting with the SAM and RNA substrate binding pockets. The disclosed compounds demonstrate remarkable advantages, including inhibition at nanomolar concentrations, excellent selectivity profiles over human methyltransferases, high antiviral activity with low cytotoxicity, and low metabolic liability. The Examples demonstrate potent and selective anti-SARS-CoV-2 activity can be achieved by targeting the Nsp14 methyltransferase and that the compounds can be used as COVID-19 therapeutics.

The compounds disclosed herein have the formula

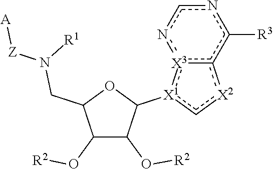

or a pharmaceutically acceptable salt or prodrug thereof,
wherein—
(i) X¹ is C, X² is C—R⁴, and X³ is N, or (ii) X¹ is N, X² is N, and X³ is C, or (iii) X¹ is C;
X² is S, X³ is C, or (iv) X¹ is N, X² is C—R⁴, and X³ is C;
R¹ is H or an alkyl;
each R² is independently selected from H, an alkyl, and a —C(O)-alkyl;
R³ is an amino or an amido;
R⁴ is H or a halo;
Q is O or —CH₂—.
Z is a sulfonyl, a carbonyl, or an alkyl; and
A is an aryl, a heteroaryl, or a biaryl, wherein the bicyclic aryl or bicyclic heteroaryl is optionally substituted in one or more positions with a halo, an alkyl optionally substituted at one or more position with a halo, an alkoxyl optionally substituted at one or more position with a halo, nitro, or cyano.
   In some instances, if X¹ is N, X² is N, and X³ is C; R² is H; R³ is amino; and Q is O, then A is not a monocyclic aryl.

In some embodiments, the compound is a C-nucleoside and X¹ is C, X² is C—R⁴, and X³ is N. The compound may comprise formula

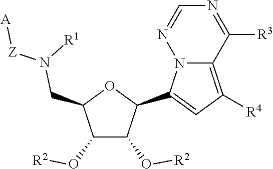

In some embodiments, X¹ is N, X² is N, and X³ is C. The compound may comprise formula

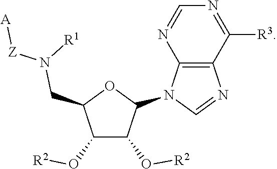

The compound may comprise formula

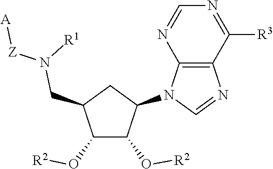

The compound may comprise formula

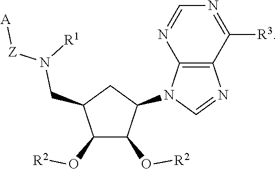

In some embodiments, X¹ is C, X² is S, and X³ is C. The compound may comprise formula

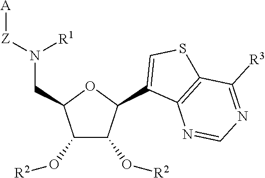

In some embodiments, X¹ is N, X² is C—R⁴, and X³ is C. The compound may comprise formula

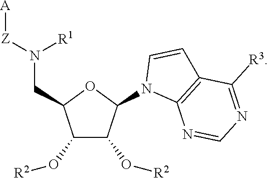

In some embodiments, Z is a sulfonyl. In other embodiments, Z is a carbonyl. In yet other embodiments, Z is an alkyl. In some embodiments, Z is —S(O)₂—, —C(O)—, or —CH₂—. In a particular embodiment, Z is —S(O)₂—.

Group A may be an aryl optionally substituted in one or more positions with a halo, an alkyl optionally substituted at one or more position with a halo, an alkoxyl optionally substituted at one or more position with a halo, nitro, or cyano. A may be a bicyclic aryl optionally substituted in one or more positions with a halo, an alkyl optionally at one or more position with a halo, an alkoxyl optionally at one or more position with a halo, nitro, or cyano. A may be a monocyclic aryl optionally substituted in one or more positions with a halo, an alkyl optionally at one or more position with a halo, an alkoxyl optionally at one or more position with a halo, nitro, or cyano. In some instances, if X¹ is N, X² is N, and X³ is C; R² is H; R³ is amino (e.g., —NH₂ or —NHCH₃); and Q is O, A is not a monocyclic aryl. Exemplary substituents include, without limitation, one or more substituents independently selected from F, Cl, Br, I, -Me, —CF₃, —OMe, —NO₂, and CN.

Group A may be a heteroaryl optionally substituted in one or more positions with a halo, an alkyl optionally at one or more position with a halo, an alkoxyl optionally at one or more position with a halo, nitro, or cyano. Exemplary substituents include, without limitation, one or more substituents independently selected from F, Cl, Br, I, -Me, —CF₃, —OMe, —NO₂, and CN. A may be a bicyclic heteroaryl optionally substituted in one or more positions with a halo, an alkyl optionally at one or more position with a halo, an alkoxyl optionally at one or more position with a halo, nitro, or cyano. Exemplary substituents include, without limitation, one or more substituents independently selected from F, Cl, Br, I, -Me, —CF₃, —OMe, —NO₂, and CN.

Group A may be a biaryl, such as biphenyl.

In some embodiments, A is a bicyclic aryl or bicyclic heteroaryl. Suitably A may be naphthyl, a quinolinyl, or a benzothiophenyl, wherein the wherein the naphthyl, quinolinyl, or the benzothiophenyl is optionally substituted in one or more positions with a halo. In particular embodiments, A is selected from:

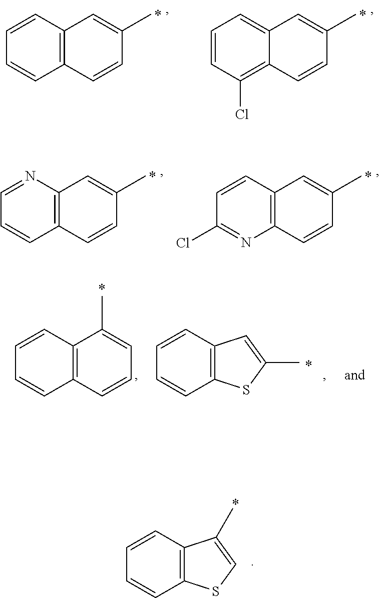

In particular embodiments, A is a naphthyl or, optionally,

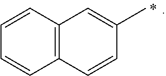

In particular embodiments, A is a halo-substituted naphthyl or, optionally,

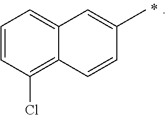

In particular embodiments, A is a benzothiophenyl or, optionally,

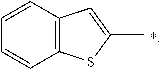

In some embodiments, A is a monocyclic aryl. Suitably A may be phenyl optionally substituted in one or more positions with alkoxyl (e.g., methoxy), nitro, cyano, or any combination thereof. In particular embodiments, A is selected from:

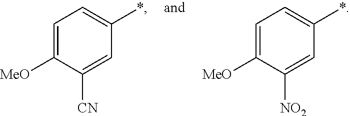

In some embodiments, Q is O. In other embodiments, Q is —CH₂—.

In some embodiments, R¹ is H. In other embodiments, R¹ is an alkyl, such as ethyl.

In some embodiments, one or both of R² are H.

In other embodiments, at least one R² is selected to form a prodrug. Prodrugs of the compounds described herein are those compounds that readily undergo chemical changes under physiological conditions to provide biologically active compounds of the present invention. Additionally, prodrugs can be converted to the compounds of the present invention by chemical or biochemical methods in an er vivo environment. Prodrugs can utilize transient protective groups to alter or eliminate undesirable properties of the biologically active compound. Prodrugs may improve bioavailability, improve selectivity, or reduce adverse or unintended side effects.

Prodrugs may comprise a moiety that can be hydrolyzed under physiological conditions to provide the biologically active compounds. Moieties that can be hydrolyzed under physiological conditions include, without limitation, esters, amides, carbamates, carbonates, ureides, and phosphates. Exemplary prodrugs include one or both R² comprising —C(O)-alkyl, such as —C(O)CH₃, or —C(O)CH(CH₃)₂. Prodrugs can be prepared using well-known methods, such as those described by 1 BURGER'S MEDICINAL CHEMISTRY AND DRUG DISCOVERY (1995) 172-178, 949-982 (Manfred E. Wolff ed., 5th ed).

In some embodiments, R³ is an amino, such as —NH₂. In other embodiments R³ is an amido, such as —NHC(O)CH₃.

In some embodiments, R⁴ is hydrogen. In other embodiments, R⁴ is halo, such as I or F.

Exemplary compounds of the present disclosure include:
(2R,3R,4S,5R)-2-(6-Amino-9H-purin-9-yl)-5-(((naphthalen-2-ylmethyl)amino)methyl)tetrahydrofuran-3,4-diol (1);
N-(((2R,3S,4R,5R)-5-(6-Amino-9H-purin-9-yl)-3,4-dihydroxytetrahydrofuran-2-yl)methyl)-2-naphthamide (2);
N-(((2R,3S,4R,5R)-5-(6-Amino-9H-purin-9-yl)-3,4-dihydroxytetrahydrofuran-2-yl)methyl)naphthalene-2-sulfonamide (3);
N-(((2R,3S,4R,5R)-5-(6-Amino-9H-purin-9-yl)-3,4-dihydroxytetrahydrofuran-2-yl)methyl)quinoline-7-sulfonamide (4);
N-(((2R,3S,4R,5R)-5-(6-Amino-9H-purin-9-yl)-3,4-dihydroxytetrahydrofuran-2-yl)methyl)-2-chloroquinoline-6-sulfonamide (5);
N-(9-((2R,3R,4S,5R)-3,4-Dihydroxy-5-((naphthalene-2-sulfonamido)methyl)tetrahydrofuran-2-yl)-9H-purin-6-yl)acetamide (6);
(2R,3R,4R,5R)-2-(6-Amino-9H-purin-9-yl)-5-((naphthalene-2-sulfonamido)methyl)tetrahydrofuran-3,4-diyl bis(2-methylpropanoate) (7)
(2R,3R,4R,5R)-2-(6-Amino-9H-purin-9-yl)-5-((naphthalene-2-sulfonamido)methyl)tetrahydrofuran-3,4-diyl diacetate (8);
(2R,3R,4R,5R)-2-(6-Acetamido-9H-purin-9-yl)-5-((naphthalene-2-sulfonamido)methyl)tetrahydrofuran-3,4-diyl diacetate (9);
N-(((2R,3S,4R,5S)-5-(4-Aminopyrrolo[2,1-f][1,2,4]triazin-7-yl)-3,4-dihydroxytetrahydrofuran-2-yl)methyl)naphthalene-2-sulfonamide (10);
(2S,3S,4R,5R)-2-(4-Aminopyrrolo[2,1-f][1,2,4]triazin-7-yl)-5-((naphthalene-2-sulfonamido)methyl)tetrahydrofuran-3,4-diyl diacetate (11);
N-(((2R,3S,4R,5S)-5-(4-Aminopyrrolo[2,1-f][1,2,4]triazin-7-yl)-3,4-dihydroxytetrahydrofuran-2-yl)methyl)naphthalene-1-sulfonamide (12);
N-(((2R,3S,4R,5S)-5-(4-aminopyrrolo[2,1-f][1,2,4]triazin-7-yl)-3,4-dihydroxytetrahydrofuran-2-yl)methyl)benzo[b]thiophene-2-sulfonamide (13);
N-(((2R,3 S,4R,5S)-5-(4-Aminopyrrolo[2,1-J][1,2,4]triazin-7-yl)-3,4-dihydroxytetrahydrofuran-2-yl)methyl)benzo[b]thiophene-3-sulfonamide (14);
N-(((1R,2R,3 S,4R)-4-(6-Amino-9H-purin-9-yl)-2,3-dihydroxycyclopentyl)methyl)naphthalene-2-sulfonamide (A1);
N-(((1R,2R,3S,4R)-4-(6-Amino-9H-purin-9-yl)-2,3-dihydroxycyclopentyl)methyl)-N-ethylnaphthalene-2-sulfonamide (A2);
N-(((1R,2S,3R,4R)-4-(6-Amino-9H-purin-9-yl)-2,3-dihydroxycyclopentyl)methyl)naphthalene-2-sulfonamide (A3);
N-(((1R,2S,3R,4R)-4-(6-Amino-9H-purin-9-yl)-2,3-dihydroxycyclopentyl)methyl)-N-ethylnaphthalene-2-sulfonamide (A4);
N-(((2R,3 S,4R,5 S)-5-(4-aminopyrrolo[2,1-f][1,2,4]triazin-7-yl)-3,4-dihydroxytetrahydrofuran-2-yl)methyl)-5-chloronaphthalene-2-sulfonamide (39);
N-(((2R,3 S,4R,5S)-5-(4-aminopyrrolo[2,1-f][1,2,4]triazin-7-yl)-3,4-dihydroxytetrahydrofuran-2-yl)methyl)-3-cyano-4-methoxybenzenesulfonamide (41);
N-(((2R,3 S,4R,5S)-5-(4-aminopyrrolo[2,1-f][1,2,4]triazin-7-yl)-3,4-dihydroxytetrahydrofuran-2-yl)methyl)-4-methoxy-3-nitrobenzenesulfonamide (43);
N-(((2R,3 S,4R,5S)-5-(4-Aminopyrrolo[2,1-f][1,2,4]triazin-7-yl)-3,4-dihydroxytetrahydrofuran-2-yl)methyl)-[1,1′-biphenyl]-3-sulfonamide (45);
N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-[1,1′-biphenyl]-4-sulfonamide (47);
N-(((2R,3S,4R,5S)-5-(4-Amino-5-iodopyrrolo[2,1-f][1,2,4]triazin-7-yl)-3,4-dihydroxytetrahydrofuran-2-yl)methyl)naphthalene-2-sulfonamide (49);
N-(((2R,3 S,4R,5S)-5-(4-Amino-5-fluoropyrrolo[2,1-f][1,2,4]triazin-7-yl)-3,4-dihydroxytetrahydrofuran-2-yl)methyl)naphthalene-2-sulfonamide (51);
N-(((2R,3S,4R,5S)-5-(4-Aminopyrrolo[2,1-][1,2,4]triazin-7-yl)-3,4-dihydroxytetrahydrofuran-2-yl)methyl)-N-ethylnaphthalene-2-sulfonamide (53);
N-(((2R,3 S,4R,5S)-5-(4-Aminothieno[3,2-d]pyrimidin-7-yl)-3,4-dihydroxytetrahydrofuran-2-yl)methyl)naphthalene-2-sulfonamide (B12);
N-(((2R,3S,4R,5R)-5-(4-Amino-7H-pyrrolo[2,3-d]pyrimidin-7-yl)-3,4-dihydroxytetrahydrofuran-2-yl)methyl)naphthalene-2-sulfonamide (C9);
N-(((2R,3 S,4R,5R)-5-(4-Amino-7H-pyrrolo[2,3-d]pyrimidin-7-yl)-3,4-dihydroxytetrahydrofuran-2-yl)methyl)-3-cyano-4-methoxybenzenesulfonamide (C11); and
N-(((2R,3S,4R,5R)-5-(4-Amino-7H-pyrrolo[2,3-d]pyrimidin-7-yl)-3,4-dihydroxytetrahydrofuran-2-yl)methyl)-3-cyano-N-ethyl-4-methoxybenzenesulfonamide (C13);
   or a pharmaceutically acceptable salt thereof.

Representative methods of making the compounds are provided in the Examples.

As used herein, an asterick “*” or a plus sign “+” may be used to designate the point of attachment for any radical group or substituent group.

The term “alkyl” as contemplated herein includes a straight-chain or branched alkyl radical in all of its isomeric forms, such as a straight or branched group of 1-12, 1-10, 1-6, or 1-4 carbon atoms, referred to herein as C₁-C₁₂ alkyl, C₁-C₁₀-alkyl, C₁-C₆-alkyl, C₁-C₄-alkyl respectively.

The term “alkylene” refers to a diradical of an alkyl group. An exemplary alkylene group is —CH₂—.

The term “haloalkyl” refers to an alkyl group that is substituted with at least one halogen. For example, —CH₂F, —CHF, —CF₃, —CH₂CF₃, —CF₂CF₃, and the like

The term “heteroalkyl” as used herein refers to an “alkyl” group in which at least one carbon atom has been replaced with a heteroatom (e.g., an O, N, or S atom). One type of heteroalkyl group is an “alkoxyl” group The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number oring carbon atoms. For example, the partially unsaturated carbocyclyl may contain 5-14, 5-12, 5-8, or 5-6 ring carbon atoms, and accordingly be referred to as a C₅-C₁₄, C₅-C₁₂, C₅-C₈, or C₅-C₆ membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

An “epoxide” is a cyclic ether with a three-atom ring typically include two carbon atoms and whose shape approximates an isosceles triangle. Epoxides can be formed by oxidation of a double bound where the carbon atoms of the double bond form an epoxide with an oxygen atom.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —R¹C(O)N(R²)—, —R¹C(O)N(R²) R³—, —C(O)N R² R³, or —C(O)NH₂, wherein R¹, R² and R³ are independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Compositions comprising substantially purified stereoisomers, epimers, or enantiomers, or analogs or derivatives thereof are contemplated herein (e.g., a composition comprising at least about 90%, 95%, or 99% pure stereoisomer, epimer, or enantiomer.)

### Pharmaceutical Compositions

The compounds disclosed herein may be formulated as pharmaceutical compositions that include: an effective amount of one or more compounds and one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The compounds utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

### Methods

Methods for treating subjects with the compounds disclosed herein are provided. Suitably the method for treating a subject comprises administering to the subject an effective amount of one or more of the compounds disclosed herein or a pharmaceutical composition comprising the effective amount of one or more of the compounds disclosed herein.

A “subject” may be interchangeable with “patient” or “individual” and means an animal, which may be a human or non-human animal, in need of treatment. A “subject in need of treatment” may include a subject having a disease, disorder, or condition that is responsive to therapy with one or more of the compounds disclosed herein. In some embodiments, the subject is responsive to therapy with one or more of the compounds disclosed herein in combination with one or more additional therapeutic agents.

The terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

The term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A “subject in need of treatment” may include a subject in need of treatment for a viral infection. The viral infection may be an infection of the subject by an enveloped, single-stranded positive-sense RNA virus of the family Coronaviridae and optionally of the genus Betacoronavirus. Exemplary viruses of the genus include SARS-CoV-2, SARS-CoV, and MERS-CoV. The effective amount of the compound administered to the subject may have the desired effect may be inhibiting the replication of the virus in the subject or to slow or reverse the progression or severity of resultant symptoms associated with the viral infection.

In some embodiments, the virus infecting the subject is SARS-CoV-2. SARS-CoV-2's RNA genome encodes many viral proteins, including four structural and 16 nonstructural proteins (Nsp1-16). The present technology targets S-adenosylmethionine (SAM)-dependent viral RNA cap methylation machinery. The RNA 5′-cap structure is methylated to ensure viral translation/replication and to evade host immune surveillance.⁷ In SARS-CoV-2, the RNA 5′-cap structure is sequentially methylated at the N7 position of the guanosine by the guanine N7-methyltransferase (N7-MTase, Nsp14)⁸ and the 2′-O position of the ribose of the first RNA nucleotide by the 2′-O-methyltransferase (2′-O-MTase, Nsp16).⁹ As a bifunctional protein, Nsp14 also contains an exoribonuclease domain,¹⁰ which is important for proofreading, mismatch excision, and drug resistance.¹¹⁻¹² While the Nsp14 ExoN activity of SARS-CoV needs to be activated through an interaction with Nsp10,¹³⁻¹⁴ such activation is not required for Nsp14 MTase.^{9, 13}

Mutational analysis of Nsp14 MTase has shown that the MTase activity is key for SARS-CoV virus replication/transcription and viability.^{8,15} Furthermore, the Nsp14 N7-MTase activity of SARS-CoV-2 is required for host translational shutdown and evasion of the innate immune response.¹⁶⁻¹⁷ Known SARS-CoV Nsp14 MTase inhibitors are either obtained from screening^{9, 18} or are adenine dinucleoside SAM analogs.¹⁹ Many inhibitors of SARS-CoV-2 Nsp14 MTase that have been identified through screening of commercially available libraries have been found to be relatively weak.²⁰⁻²² There are potent S-adenosylhomocysteine (SAH)/SAM analogs that have been identified from a collection of other SAM-dependent MTase inhibitors²³ or rationally designed.²⁴⁻²⁵ However, most of these inhibitors are highly polar and no anti-SARS-CoV-2 activity has been reported. In addition, virtual screenings have been performed to identify potential Nsp14 MTase inhibitors.²⁶⁻²⁸

Structural studies have revealed that the MTase domain of the SARS-CoV Nsp14 protein is distinct from the canonical Rossmann fold due to a rare β-sheet insertion and a peripheral zinc finger.^{12, 29} Indeed, the MTase domain of the SARS-CoV Nsp14-Nsp10 complex (PDBs 5C8S and 5C8T) features a uniquely constricted SAM and RNA substrate binding site, in which the guanine ring of the cap structure (GpppA) is placed in a narrow pocket and positioned in close proximity to SAM for the methyl group transfer. Newly reported cryo-EM structures of SARS-CoV-2 Nsp14 MTase,³⁰⁻³¹ albeit in an apo form, also have a similarly constrained MTase active site.

This constricted spatial arrangement allows for bi-substrate inhibitors of Nsp14 MTase, such as those compounds disclosed herein. Bi-substrate inhibitors generally entail structural moieties from two substrates involved in a biochemical reaction. Nsp14 MTase brings together SAM and an RNA substrate and transfers a methyl group from SAM to the N7 atom of the guanine. The disclosed compounds comprise an adenosine moiety or adenosine-like moiety from SAM. To mimic the guanine ring moiety of the RNA substrate, a bicyclic aromatic ring, such as naphthyl, may be used. The adenosine moiety and the aromatic ring were connected via a structurally varied linker.

Methods of inhibiting viral replication are also provided. The method comprises contacting a cellular medium having a virus therein with an effective amount of a compound described herein.

In some embodiments, the methods described herein are practiced in vivo. In other embodiments, the methods described herein are practiced in vitro or ex vivo.

### Abbreviations

SARS-CoV-2, severe acute respiratory syndrome coronavirus-2; SARS-CoV, severe acute respiratory syndrome coronavirus; COVID-19, coronavirus disease 2019; MERS-CoV, Middle East respiratory syndrome coronavirus; Nsp, nonstructural protein; RdRp, RNA-dependent RNA polymerase; SAM, S-adenosylmethionine; MTase, methyltransferase; N7-MTase, guanine N7-methyltransferase; 2′-O-MTase, 2′-O-methyltransferase; SAH, S-adenosylhomocysteine; SAR, structure-activity relationship; IF, immunofluorescence; TI, therapeutic index; ADME, absorption, distribution, metabolism, and excretion; PAMPA, parallel artificial membrane permeability assay; NADPH, nicotinamide adenine dinucleotide phosphate; UDPGA, uridine 5′-diphosphoglucuronic acid; DPPA, diphenylphosphoryl azide; HMBC, heteronuclear multiple bond correlation.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

A biochemical assay was established in which an LC-MS/MS method was used to monitor the production of SAH, the byproduct of the methylation reaction. This assay offered an unbiased method to evaluate a compound's inhibitory activity. Our initial attempts to use alternative SAH detecting methods, such as an AptaFluor SAH MTase assay kit, showed difficulty in differentiating SAH from our adenosine-derived inhibitors, leading to erratic results. Sinefungin (IC₅₀=260 nM, Table 1), a potent pan-MTase inhibitor, was used as a control and benchmark for MTase inhibition.

SAR studies were initiated by exploring several common functionalities as potential linkers (Table 1). First, when a secondary amine was used to connect the adenosine moiety and 2-naphthalene, the resulting compound 1 had an IC₅₀ value of 9.6 μM, demonstrating the feasibility of designing bi-substrate inhibitors of Nsp14 MTase. Compound 2, which contained an amide linker, displayed enhanced inhibitory activity (IC₅₀=2.7 μM). Remarkably, installation of a sulfonamide functionality as a linker led to compound 3, which was >150-fold more active than compound 1. With an IC₅₀ value of 61 nM, compound 3 was a promising starting point for further SAR studies. To that end, the effect of 2-naphthalene modifications was examined. Replacement with a 7-quinoline ring gave compound 4, which showed inhibitory activity comparable to that of compound 1, suggesting that this minor structural change was tolerated. However, use of a 6-(2-chloroquinoline) ring (5, Table 1) led to a 7-fold reduction in inhibitory activity, probably due to the relatively bulky chloro group. Furthermore, N6-acetylated compound 6 was prepared, which had an IC₅₀ value of 48 nM, suggesting that acylation at N6 was tolerated. Taken together, the Examples proved that highly active bi-substrate inhibitors of SARS-CoV-2 Nsp14 methyltransferase could be obtained by connecting the adenosine moiety and a simply aromatic ring via a sulfonamide linker.

To assess the anti-SARS-CoV-2 activity of these compounds, A549 cells over-expressing ACE2 and TMPRSS2 (A549 AT) were used in an immunofluorescence (IF)-based assay. Remdesivir,³² an FDA-approved polymerase inhibitor, was used as a control. As expected, sinefungin with a polar amino acid side chain failed to show any activity. Disappointingly, compounds 1-5 at 50 μM showed no significant antiviral activity, likely due to poor cell permeability resulting from high polarity of sulfonamides and nucleosides. To mitigate high polarity and improve cell permeability, compound 7 was prepared, compound 3's di-isobutyrate prodrug (Table 1). While compound 7 showed antiviral activity, it was difficult to determine an EC₅₀ value because of compound 7's low solubility. As a result, a less lipophilic diacetate prodrug 8 was prepared, which was more soluble and possessed moderate antiviral activity (EC₅₀=53 μM). Compound 9 was also prepared, compound 6's diacetate prodrug, which exhibited antiviral activity similar to that of compound 8. In addition, both compounds showed minimal cytotoxicity at 100 μM. Taken together, the results demonstrate antiviral activity via a prodrug approach.

One drawback of nucleoside/nucleotide-based inhibitors is their metabolically labile glycosidic bond. To address this issue, C-nucleosides³⁴ were examined in which the glycosidic bond is replaced by a metabolically stable C—C bond. Compound 10 was prepared, in which a pyrrolo[2,1-f][1,2,4]triazin-4-amine ring (the nucleobase of remdesivir), was used as a nucleobase. Compound 10 possessed an IC₅₀ value comparable to that of 3, suggesting that C-nucleosides were well tolerated. Strikingly, compound 10 exhibited excellent antiviral activity (EC₅₀=0.72 μM) with a therapeutic index (TI=CC₅₀/EC₅₀) greater than 139 (Table 1). Surprisingly, compound 11, the diacetate prodrug of 10, was less active and more toxic than 10. Taken together, these results suggested that metabolic stability played a crucial role in antiviral activity and that the impact of the prodrug might depend on the nature of the parent nucleosides. These observations will be examined in future SAR studies.

To evaluate selectivity over human MTases, compound 3 was screened against a panel of 10 human MTases including histone lysine, protein arginine, DNA and RNA MTases. At 10 μM, compound 3 showed minimal inhibition of this diverse set of human MTases (Table S1), indicative of an excellent selectivity profile. These preliminary results bolstered our premise that it was feasible to design selective Nsp14 MTase because of its unique structure in contrast to the canonical Rossmann fold.

To identify factors that might contribute to compound 10's improved antiviral activity, compounds 3 and 10 were assessed for their in vitro absorption, distribution, metabolism, and excretion (ADME) properties including plasma stability, liver S9 stability and permeability in parallel artificial membrane permeability assay (PAMPA) (Table 2). Liver S9 fraction was used because the liver is the predominant site of drug metabolism and the S9 fraction contains both microsomal and cytosolic enzymes, including a wide variety of Phase I and Phase II enzymes. To facilitate metabolic transformations, nicotinamide adenine dinucleotide phosphate (NADPH) and uridine 5′-diphosphoglucuronic acid (UDPGA) were supplemented as cofactors in Phase I and II transformations, respectively. Compound 3 was remarkably stable in mouse and human plasma. It was also resistant to phase I and II transformations in both human and mouse liver S9 fractions. In comparison, compound 10 was similarly stable in plasma and liver S9 fractions except that it was surprisingly labile in the mouse S9 phase I reactions. This metabolic instability was likely linked to compound 10's unnatural pyrrolo[2,1-f][1,2,4]triazin-4-amine nucleobase. Nonetheless, to our best knowledge no major metabolic liabilities associated with this nucleobase in the context of remdesivir have been reported. Compounds 3 and 10 were also assessed in PAMPA, which revealed that their passive diffusion rates were equally low. Metabolic profiling indicated that neither compound suffered from major metabolic liabilities in human plasma and liver S9 fractions. In particular, compound 10 will serve as an excellent template to further improve antiviral activity and to mitigate metabolic liabilities.

To gain insight into key protein-ligand interactions that contributed to inhibitory activity, compounds were docked into the MTase domain of the SARS-CoV-2 Nsp14 structure (PDB 7EGQ). For compound 3, its adenine ring was sandwiched between Cys387 and Phe367, and formed two hydrogen bonds with Tyr368. Furthermore, the 3′-hydroxyl group of the ribose ring had an additional hydrogen bond with Asp352. At the same time, the sulfonamide linker projected the naphthalene ring into the guanine (of RNA substrate) binding pocket and allowed it to engage a π-π stacking with Phe426, a residue that is important for Nsp14 MTase activity. Remarkably, Phe426 formed a π-π interaction with the guanine ring of GpppA (an RNA substrate mimic) in PDB 5C8S, a SARS-CoV Nsp14 cocrystal structure in which both GpppA and SAH were present in the MTase active site. Thus, this binding mode supported our design strategy in which a simple 2-naphthalene was used to mimic the guanine ring of the RNA substrate. Docking of compound 10 revealed that its pyrrolo[2,1-f][1,2,4]triazin-4-amine nucleobase maintained the hydrogen bonds elicited by the original adenine ring. However, examination of the top pose revealed no apparent π-π interaction between the naphthalene ring and Phe426 even though compound 10 displayed a binding configuration very similar to compound 3. Instead, the sulfonamide hydrogen formed a hydrogen bond with Asn386, a key residue for N7-methylation as judged by mutation analysis.^{15, 29} Interestingly, a similar interaction (between the amide hydrogen of compound 2 and Asn386) was also identified in the top pose of compound 2. Because compounds 3 and 10 possessed similar biochemical IC₅₀ values (Table 1) and docking scores (−10.4 and −10.5 for compounds 3 and 10, respectively), our docking experiment suggested that a π-π interaction with Phe426 and a hydrogen bond with Asn386 were key contributors to the enzymatic activity and therefore will be further investigated in our future SAR efforts. In contrast to compounds 1-5, docking of compound 6 produced no pose in which its acetylated adenine ring interacted with the protein via hydrogen bonding with Tyr368. This finding was not unexpected because the narrow adenine binding groove was incapable of accommodating the bulkier acetylated adenine ring of compound 6. An alternative pose yielded a poor docking score of −6.32, which apparently could not account for the high biochemical inhibitory activity of compound 6.

In summary, the uniquely constricted active site of SARS-CoV-2 Nsp14 MTase has allowed us to design bi-substrate inhibitors, which simultaneously engage residues in the SAM and RNA substrate binding pockets. Our preliminary effort has led to nanomolar inhibitors including 3 and 10, validating our bi-substrate drug design. Successful identification of potent inhibitors has also demonstrated that it is feasible to obtain inhibitors devoid of a polar amino acid moiety. Furthermore, a brief metabolic profiling of these two compounds suggests that they are less likely to have major metabolic liabilities. Moreover, compound 3, as a representative of these bi-substrate inhibitors, displays an excellent selectivity profile over a panel of human MTases. Remarkably, use of prodrugs has led to compound 8, which showed significant, albeit moderate, antiviral activity. C-nucleoside 10, which exhibits high antiviral activity and low cytotoxicity, clearly proving that superior anti-SARS-CoV-2 inhibition can be accomplished by targeting the Nsp14 MTase. The compounds are believed to be the first bi-substrate inhibitors that have been specifically designed to target the SARS-CoV-2 Nsp14 MTase. Among them, compound 10 is the first designed SARS-CoV-2 Nsp14 MTase inhibitor that has achieved sub-micromolar antiviral activity without significant cytotoxicity. Therefore, the disclosed compounds represent a substantial advancement in Nsp14 MTase drug discovery and allows for the use of Nsp14 MTase inhibitors as COVID-19 therapeutics.

### REFERENCES

1. Li, J.; Lai, S.; Gao, G. F.; Shi, W., The emergence, genomic diversity and global spread of SARS-CoV-2. Nature 2021, 600 (7889), 408-418.
2. https://coronavirus.jhu.edu/map.html.
3. Su, H.; Zhou, F.; Huang, Z.; Ma, X.; Natarajan, K.; Zhang, M.; Huang, Y.; Su, H., Molecular Insights into Small-Molecule Drug Discovery for SARS-CoV-2. Angew. Chem. hit. Ed. Engl. 2021, 60 (18), 9789-9802.
4. Gao, S.; Huang, T.; Song, L.; Xu, S.; Cheng, Y.; Cherukupalli, S.; Kang, D.; Zhao, T.; Sun, L.; Zhang, J.; Zhan, P.; Liu, X., Medicinal chemistry strategies towards the development of effective SARS-CoV-2 inhibitors. Acta Pharm Sin B 2022, 12 (2), 581-599.
5. Pillaiyar, T.; Wendt, L. L.; Manickam, M.; Easwaran, M., The recent outbreaks of human coronaviruses: A medicinal chemistry perspective. Med. Res. Rev. 2021, 41 (1), 72-135.
6. Decroly, E.; Ferron, F.; Lescar, J.; Canard, B., Conventional and unconventional mechanisms for capping viral mRNA. Nat Rev Microbiol 2011, 10 (1), 51-65.
7. Ferron, F.; Decroly, E.; Selisko, B.; Canard, B., The viral RNA capping machinery as a target for antiviral drugs. Antiviral Res. 2012, 96 (1), 21-31.
8. Chen, Y.; Cai, H.; Pan, J.; Xiang, N.; Tien, P.; Ahola, T.; Guo, D., Functional screen reveals SARS coronavirus nonstructural protein nsp14 as a novel cap N7 methyltransferase. Proc. Natl. Acad. Sci. U.S.A 2009, 106 (9), 3484-9.
9. Bouvet, M.; Debarnot, C.; Imbert, I.; Selisko, B.; Snijder, E. J.; Canard, B.; Decroly, E., In vitro reconstitution of SARS-coronavirus mRNA cap methylation. PLoS Pathog 2010, 6 (4), e1000863.
10. Minskaia, E.; Hertzig, T.; Gorbalenya, A. E.; Campanacci, V.; Cambillau, C.; Canard, B.; Ziebuhr, J., Discovery of an RNA virus 3′->5′ exoribonuclease that is critically involved in coronavirus RNA synthesis. Proc. Natl. Acad. Sci. U.S.A 2006, 103 (13), 5108-13.
11. Agostini, M. L.; Andres, E. L.; Sims, A. C.; Graham, R. L.; Sheahan, T. P.; Lu, X.; Smith, E. C.; Case, J. B.; Feng, J. Y.; Jordan, R.; Ray, A. S.; Cihlar, T.; Siegel, D.; Mackman, R. L.; Clarke, M. O.; Baric, R. S.; Denison, M. R., Coronavirus Susceptibility to the Antiviral Remdesivir (GS-5734) Is Mediated by the Viral Polymerase and the Proofreading Exoribonuclease. mBio 2018, 9 (2).
12. Ferron, F.; Subissi, L.; Silveira De Morais, A. T.; Le, N. T. T.; Sevajol, M.; Gluais, L.; Decroly, E.; Vonrhein, C.; Bricogne, G.; Canard, B.; Imbert, I., Structural and molecular basis of mismatch correction and ribavirin excision from coronavirus RNA. Proc. Natl. Acad. Sci. U.S.A. 2018, 115 (2), E162-E171.
13. Bouvet, M.; Imbert, I.; Subissi, L.; Gluais, L.; Canard, B.; Decroly, E., RNA 3′-end mismatch excision by the severe acute respiratory syndrome coronavirus nonstructural protein nsp10/nsp14 exoribonuclease complex. Proc. Natl. Acad. Sci. U.S.A 2012, 109 (24), 9372-7.
14. Bouvet, M.; Lugari, A.; Posthuma, C. C.; Zevenhoven, J. C.; Bernard, S.; Betzi, S.; Imbert, I.; Canard, B.; Guillemot, J. C.; Lecine, P.; Pfefferle, S.; Drosten, C.; Snijder, E. J.; Decroly, E.; Morelli, X., Coronavirus Nsp10, a critical co-factor for activation of multiple replicative enzymes. J. Biol. Chem. 2014, 289 (37), 25783-96.
15. Ogando, N. S.; El Kazzi, P.; Zevenhoven-Dobbe, J. C.; Bontes, B. W.; Decombe, A.; Posthuma, C. C.; Thiel, V.; Canard, B.; Ferron, F.; Decroly, E.; Snijder, E. J., Structure-function analysis of the nsp14 N7-guanine methyltransferase reveals an essential role in Betacoronavirus replication. Proc. Natl. Acad. Sci. U.S.A 2021, 118 (49).
16. Hsu, J. C.; Laurent-Rolle, M.; Pawlak, J. B.; Wilen, C. B.; Cresswell, P., Translational shutdown and evasion of the innate immune response by SARS-CoV-2 NSP14 protein. Proc. Natl. Acad. Sci. U S. A. 2021, 118 (24).
17. Pan, R.; Kindler, E.; Cao, L.; Zhou, Y.; Zhang, Z.; Liu, Q.; Ebert, N.; Zust, R.; Sun, Y.; Gorbalenya, A. E.; Perlman, S.; Thiel, V.; Chen, Y.; Guo, D., N7-Methylation of the Coronavirus RNA Cap Is Required for Maximal Virulence by Preventing Innate Immune Recognition. mBio 2022, e0366221.
18. Aouadi, W.; Eydoux, C.; Coutard, B.; Martin, B.; Debart, F.; Vasseur, J. J.; Contreras, J. M.; Morice, C.; Querat, G.; Jung, M. L.; Canard, B.; Guillemot, J. C.; Decroly, E., Toward the identification of viral cap-methyltransferase inhibitors by fluorescence screening assay. Antiviral Res. 2017, 144, 330-339.
19. Ahmed-Belkacem, R.; Sutto-Ortiz, P.; Guiraud, M.; Canard, B.; Vasseur, J. J.; Decroly, E.; Debart, F., Synthesis of adenine dinucleosides SAM analogs as specific inhibitors of SARS-CoV nsp14 RNA cap guanine-N7-methyltransferase. Eur J Med Chem 2020, 201, 112557.
20. Basu, S.; Mak, T.; Ulferts, R.; Wu, M.; Deegan, T.; Fujisawa, R.; Tan, K. W.; Lim, C. T.; Basier, C.; Canal, B.; Curran, J. F.; Drury, L. S.; McClure, A. W.; Roberts, E. L.; Weissmann, F.; Zeisner, T. U.; Beale, R.; Cowling, V. H.; Howell, M.; Labib, K.; Diffley, J. F. X., Identifying SARS-CoV-2 antiviral compounds by screening for small molecule inhibitors of Nsp14 RNA cap methyltransferase. Biochem. J. 2021, 478 (13), 2481-2497.
21. Kasprzyk, R.; Spiewla, T. J.; Smietanski, M.; Golojuch, S.; Vangeel, L.; De Jonghe, S.; Jochmans, D.; Neyts, J.; Kowalska, J.; Jemielity, J., Identification and evaluation of potential SARS-CoV-2 antiviral agents targeting mRNA cap guanine N7-Methyltransferase. Antiviral Res. 2021, 193, 105142.
22. Pearson, L. A.; Green, C. J.; Lin; Petit, A. P.; Gray, D. W.; Cowling, V. H.; Fordyce, E. A. F., Development of a High-Throughput Screening Assay to Identify Inhibitors of the SARS-CoV-2 Guanine-N7-Methyltransferase Using RapidFire Mass Spectrometry. SLAS Discov 2021, 26 (6), 749-756.
23. Devkota, K.; Schapira, M.; Perveen, S.; Khalili Yazdi, A.; Li, F.; Chau, I.; Ghiabi, P.; Hajian, T.; Loppnau, P.; Bolotokova, A.; Satchell, K. J. F.; Wang, K.; Li, D.; Liu, J.; Smil, D.; Luo, M.; Jin, J.; Fish, P. V.; Brown, P. J.; Vedadi, M., Probing the SAM Binding Site of SARS-CoV-2 Nsp14 In Vitro Using SAM Competitive Inhibitors Guides Developing Selective Bisubstrate Inhibitors. SLAS Discov 2021, 26 (9), 1200-1211.
24. Otava, T.; Sala, M.; Li, F.; Fanfrlik, J.; Devkota, K.; Perveen, S.; Chau, I.; Pakarian, P.; Hobza, P.; Vedadi, M.; Boura, E.; Nencka, R., The Structure-Based Design of SARS-CoV-2 nsp14 Methyltransferase Ligands Yields Nanomolar Inhibitors. ACS Infect Dis 2021, 7 (8), 2214-2220.
25. Bobileva, O.; Bobrovs, R.; Kanepe, I.; Patetko, L.; Kalnins, G.; Sisovs, M.; Bula, A. L.; Gri Nberga, S.; Boroduskis, M. R.; Ramata-Stunda, A.; Rostoks, N.; Jirgensons, A.; Ta Rs, K.; Jaudzems, K., Potent SARS-CoV-2 mRNA Cap Methyltransferase Inhibitors by Bioisosteric Replacement of Methionine in SAM Cosubstrate. ACS medicinal chemistry letters 2021, 12 (7), 1102-1107.
26. Bobrovs, R.; Kanepe, I.; Narvaiss, N.; Patetko, L.; Kalnins, G.; Sisovs, M.; Bula, A. L.; Grinberga, S.; Boroduskis, M.; Ramata-Stunda, A.; Rostoks, N.; Jirgensons, A.; Tars, K.; Jaudzems, K., Discovery of SARS-CoV-2 Nsp14 and Nsp16 Methyltransferase Inhibitors by High-Throughput Virtual Screening. Pharmaceuticals (Basel) 2021, 14 (12).
27. Gorgulla, C.; Padmanabha Das, K. M.; Leigh, K. E.; Cespugli, M.; Fischer, P. D.; Wang, Z. F.; Tesseyre, G.; Pandita, S.; Shnapir, A.; Calderaio, A.; Gechev, M.; Rose, A.; Lewis, N.; Hutcheson, C.; Yaffe, E.; Luxenburg, R.; Herce, H. D.; Durmaz, V.; Halazonetis, T. D.; Fackeldey, K.; Patten, J. J.; Chuprina, A.; Dziuba, I.; Plekhova, A.; Moroz, Y.; Radchenko, D.; Tarkhanova, O.; Yavnyuk, I.; Gruber, C.; Yust, R.; Payne, D.; Naar, A. M.; Namchuk, M. N.; Davey, R. A.; Wagner, G.; Kinney, J.; Arthanari, H., A multi-pronged approach targeting SARS-CoV-2 proteins using ultra-large virtual screening. iScience 2021, 24 (2), 102021.
28. Selvaraj, C.; Dinesh, D. C.; Panwar, U.; Abhirami, R.; Boura, E.; Singh, S. K., Structure-based virtual screening and molecular dynamics simulation of SARS-CoV-2 Guanine-N7 methyltransferase (nsp14) for identifying antiviral inhibitors against COVID-19. J. Biomol. Struct. Dyn. 2021, 39 (13), 4582-4593.
29. Ma, Y.; Wu, L.; Shaw, N.; Gao, Y.; Wang, J.; Sun, Y.; Lou, Z.; Yan, L.; Zhang, R.; Rao, Z., Structural basis and functional analysis of the SARS coronavirus nsp14-nsp10 complex. Proc. Natl. Acad. Sci. U.S.A 2015, 112 (30), 9436-41.
30. Yan, L.; Yang, Y.; Li, M.; Zhang, Y.; Zheng, L.; Ge, J.; Huang, Y. C.; Liu, Z.; Wang, T.; Gao, S.; Zhang, R.; Huang, Y. Y.; Guddat, L. W.; Gao, Y.; Rao, Z.; Lou, Z., Coupling of N7-methyltransferase and 3′-5′ exoribonuclease with SARS-CoV-2 polymerase reveals mechanisms for capping and proofreading. Cell 2021, 184 (13), 3474-3485 el1.
31. Liu, C.; Shi, W.; Becker, S. T.; Schatz, D. G.; Liu, B.; Yang, Y., Structural basis of mismatch recognition by a SARS-CoV-2 proofreading enzyme. Science 2021, 373 (6559), 1142-1146.
32. Wang, M.; Cao, R.; Zhang, L.; Yang, X.; Liu, J.; Xu, M.; Shi, Z.; Hu, Z.; Zhong, W.; Xiao, G., Remdesivir and chloroquine effectively inhibit the recently emerged novel coronavirus (2019-nCoV) in vitro. Cell Res. 2020, 30 (3), 269-271.
33. Dousson, C. B., Current and future use of nucleo(s)tide prodrugs in the treatment of hepatitis C virus infection. Antivir. Chem. Chemother. 2018, 26, 2040206618756430.
34. Stambasky, J.; Hocek, M.; Kocovsky, P., C-nucleosides: synthetic strategies and biological applications. Chem. Rev. 2009, 109 (12), 6729-64.
35. Chen, X.; Murawski, A.; Patel, K.; Crespi, C. L.; Balimane, P. V., A novel design of artificial membrane for improving the PAMPA model. Pharm. Res. 2008, 25 (7), 1511-20.
36. Xie, J.; Wang, Z., Can remdesivir and its parent nucleoside GS-441524 be potential oral drugs? An in vitro and in vivo DMPK assessment. Acta Pharm Sin B 2021, 11 (6), 1607-1616.
37. Liu, F.; Austin, D. J., Synthesis of 5′-functionalized adenosine: suppression of cyclonucleoside formation. Tetrahedron Lett. 2001, 42 (18), 3153-3154.
38. von Keutz, T.; Williams, J. D.; Kappe, C. O., Continuous Flow C-Glycosylation via Metal-Halogen Exchange: Process Understanding and Improvements toward Efficient Manufacturing of Remdesivir. Org Process Res Dev 2020, 24 (10), 2362-2368.
39. Metobo, S. E.; Xu, J.; Saunders, O. L.; Butler, T.; Aktoudianakis, E.; Cho, A.; Kim, C. U., Practical synthesis of 1′-substituted Tubercidin C-nucleoside analogs. Tetrahedron Lett. 2012, 53 (5), 484-486.
40. Li, Q.; Lescrinier, E.; Groaz, E.; Persoons, L.; Daelemans, D.; Herdewijn, P.; De Jonghe, S., Synthesis and Biological Evaluation of Pyrrolo[2,1-f][1,2,4]triazine C-Nucleosides with a Ribose, 2′-Deoxyribose, and 2′,3′-Dideoxyribose Sugar Moiety. ChemMedChem 2018, 13 (1), 97-104.

### Materials and Methods

### SARS-CoV-2 Nsp14 MTase Assay

Protein expression and purification. The plasmid pCDFDuet-1-nsp14S2 was transformed into E. coli BL21(DE3) STAR for expression. All media was supplemented with 50 μg/mL streptomycin for plasmid maintenance. Overnight cultures were used to inoculate 800 mL LB, 1% final concentration in a 3 L Fernbach flask. The culture was grown at 37° C. 220 RPM to an OD600 0.6. The flask was moved to 4° C. for 10 minutes and then induced with IPTG to a final concentration of 0.4 mM. The culture was incubated overnight at 18° C. and 220 RPM. Cultures were centrifuged and the cell pellet was frozen at −80° C. until protein isolation.

The cell pellet was sonicated 4 times 2 min using a power setting of 8 and 30% duty cycle on a Branson 250 Sonifier (Brookfield, CT, USA in 40 mL of lysis buffer (50 mM Tris pH 8.0, 200 mM NaCl, 20 mM imidazole). Cell debris was removed by centrifugation at 45000 g for 15 min at 4° C. The protein was purified using a HisTrap HP 5 mL column (Cytiva) on a Bio-Rad NGS system (Hercules, CA, USA). Protein was eluted using a linear gradient from buffer A (50 mM Tris pH 8.0, 200 mM NaCl, 5 mM imidazole) to buffer B (50 mM Tris pH 8.0, 200 mM NaCl, 500 mM imidazole). The purified protein was concentrated using an Amicon Ultra 15 3,000 MWCO filter and desalted using a PD-10 column into storage buffer (10 mM HEPES pH 7.5, 150 mM NaCl). Glycerol was added to 50% and the protein was stored at −20° C.

MTase assay. The assay was carried out generally following the procedures reported previously.¹ Assays were set up in 1.5 mL centrifuge tubes at a final volume of 50 μL and consisted of 40 mM Tris pH 8.0, 0.5 mM DTT, 2 mM MgCl₂, 100 nM cAMP (mass spec internal standard), 3 μM GpppA, and 10 nM Nsp14. A negative control reaction was included that lacked GpppA. Reactions were preincubated with DMSO or inhibitors (0.5 L) for 15 min at room temperature and initiated by adding SAM to 0.4 μM. Reactions were incubated for 15 min at 30° C. and quenched with 3 volumes (150 μL) acetonitrile.

LC/MS/MS method. The acetonitrile quenched samples were analyzed using a LC/MS/MS system consisting of an AB Sciex QTrap 5500 mass spectrometer and an Agilent 1260 Infinity HPLC (Santa Clara, CA, USA). Chromatographic separation of the analytes was achieved on a Thermo Aquasil C18 column (150×2.1 mm, 3 μm) using two eluents: (A) water with 0.10% formic acid, and (B) acetonitrile with 0.1% formic acid. The mobile phase was delivered at a flow rate of 0.3 mL/min with a gradient of A and B as follows: 0-2.3 min, 5-30% B (v/v); 2.3-2.8 min, 30% B (v/v); 2.8-3.0 min, 30-5% B (v/v); 3.0-6.0 min, 5% B (v/v). MS/MS detection of SAH and SAM was conducted using positive electrospray ionization with mass transitions of m/z 385.2→136.0 and m/z 399.2→250.1, respectively. Cyclic AMP (cAMP, m/z 330.0→136.0) was used as an internal standard. Quantitation of SAH and SAM were performed using matrix-matched 7-points standard curves. SAH concentrations were normalized to the cAMP signal and data was fit using GraphPad Prism (GraphPad Software; San Diego, CA, USA) to determine IC₅₀ values.

Detection using MTase-Glo™ methyltransferase assay kits. The assay was run according to Promega MTase-Glo™ kit (V7601) instructions. Nsp14 was used at a final concentration of 25 nM/reaction. GpppA was purchased by New England Biolabs and used at a final concentration of 3 uM/reaction. Sinefungin control was purchased from Abcam (ab144518). The assay was run in a low volume 384-well white plate as suggested by Promega. Compounds were tested at final concentrations of 10 μM and 1 μM. Compounds, Nsp14, GpppA at MTase-Glo™ reagent were pre-incubated for 15 min at room temperature. The assay was started with addition of SAM at final concentration of 1 uM/reaction (negative control had no SAM). The assay plate was mixed well with a plate shaker after each addition. The reaction was incubated for 15 min at 30° C. MTase-Glo™ detection solution was added, and the plate was incubated 60 min at room temperature. Luminescence was read on an M5e spectrophotometer.

### Cells and Viruses

A549/ACE2/TMPRSS2 cells² (provided by M. Saeed, Boston University; Boston, MA, USA) were maintained in Dulbecco's modified Eagle's medium (DMEM) high glucose supplemented with 10% fetal bovine serum (FBS), 100 IU streptomycin/penicillin per mL, 10 mM HEPES, 1× Non-Essential Amino Acids (NEAA), 1× Glutamax, 1 mM sodium pyruvate, 5 μg/mL plasmocin, 0.5 μg/mL puromycin and 0.5 μg/mL blasticidin). SARS-CoV-2 isolate USA-WA1/2020 (NR-52281) was obtained through BEI Resources (Manasses, VA, USA), and propagated in Vero-E6 cells.

### SARS-CoV-2 Immunofluorescence Antiviral Assay

1.5×10⁴ A549/ACE2/TMPRSS2 cells per well were plated in a 96 well plate. Next day, the medium was replaced with 50 μL of SARS-CoV-2 infection medium (DMEM high glucose supplemented with 5% FBS, 100 IU streptomycin/penicillin per mL, 10 mM HEPES, 1×NEAA, 1× Glutamax and 1 mM sodium pyruvate). Cells were treated with compound in 50 μL infection medium, transferred to the BSL-3 facility and inoculated with 50 μL infection medium containing SARS-CoV-2 at an MOI of 0.01. Forty-eight hours post infection, the cells were fixed with 4% PFA in 1×PBS for 30 min and processed for immunofluorescence using an anti-SARS-CoV-2 nucleoprotein antibody (Sino Biologicals 40588-T62; Wayne, PA, USA) as described.³ Images were acquired using a BioTek Cytation 1™ imaging reader (Winooski, VT, USA) and the total number of DAPI-stained cells and infected cells for each well were quantified in Gen5 software.

Percentage of infected cells was determined by dividing the number of infected cells by the total number of cells per well. The percentage of infected cells and total number of cells for each treatment was plotted into GraphPad prism (GraphPad Software; San Diego, CA, USA) to perform a non-linear regression analysis, generate infectious dose-response curves and to calculate EC₅₀ and CC₅₀ values.

### Cell Viability Assay

Viability assays for the non-infected A549/ACE2/TMPRSS2 cells were performed using the 3-(4,5-dimethylthiazol-2-yl)-5-(3-carboxymethoxyphenyl)-2-(4-sulfophenyl)-2H-tetrazolium (MTS)-based tetrazolium reduction CellTiter 96 Aqueous Non-Radioactive cell proliferation assay (Promega G5430; Madison, WI) as described. Assays were conducted using parallel plates to those used in the immunofluorescence assay. Experimental details (cell line, number of cells seeded and incubation time) corresponded to those used in the immunofluorescence assay above. The data obtained were used to calculate the CC₅₀ value using GraphPad Prism software.

### Human MTase Screening

Assay conditions. The human methyltransferase inhibition assays were performed at BPS Bioscience (San Diego, CA, USA). All of the enzymatic reactions were conducted in duplicate at room temperature for 60-960 min in a 50 μL mixture containing proper methyltransferase assay buffer, S-adenosylmethionine (SAM), enzyme, and the test compound. These 50 μL reactions were carried out in wells of a substrate pre-coated plate. First, enzyme and inhibitor were added to assay wells and preincubated for 30 min at room temperature, then SAM was added to start the reaction. Final DMSO concentration was 1% in all reactions.

After enzymatic reactions, the reaction mixtures were discarded and each of the wells was washed three times with TBST buffer, and slowly shaken with Blocking Buffer for 10 min. Wells were emptied, and 100 μL of diluted primary antibody was added. The plate was then slowly shaken for 60 minutes at room temperature. As before, the plate was emptied and washed three times with TBST, and shaken with Blocking Buffer for 10 minutes at room temperature. After discarding the Blocking Buffer, 100 μL of diluted secondary antibody was added. The plate was then slowly shaken for 30 min at room temperature. As before, the plate was emptied and washed three times with TBST, and shaken with Blocking Buffer for 10 minutes at room temperature. Blocking Buffer was discarded and a mixture of the HRP chemiluminescent substrates was freshly prepared. 100 μL of this mixture was added to each empty well. Immediately, the luminescence of the samples was measured in a BioTek Synergy™ 2 (Winooski, VT, USA) microplate reader using Gen5 software.

Data analysis. Enzyme activity assays were performed in duplicates at each concentration. The luminescence data/alpha counts were analyzed and compared. In the absence of the compound, the intensity (Cₑ) in each data set was defined as 100% activity. In the absence of enzyme, the intensity (C₀) in each data set was defined as 0% activity. The percent activity in the presence of each compound was calculated according to the following equation: % activity=(C−C₀)/(Cₑ−C₀), where C=the luminescence/alpha counts in the presence of the compound.

In vitro Metabolic Stabilities and Permeability

Plasma stability assay. The plasma stability assay was performed in triplicate by incubating each selected compound (1 μmol/L final concentration) in normal mouse (CD-1) or human plasma (Innovative Research; Novi, MI, USA) diluted to 80% with 0.1 mol/L potassium phosphate buffer (pH 7.4) at 37° C. At 0, 1, 3, 6, and 24 h, a 50 μL aliquot of the plasma mixture was taken and quenched with 150 μL of acetonitrile containing 0.1% formic acid. The samples were then vortexed and centrifuged at 15,000 rpm (Eppendorf centrifuge 5424R; Enfield, CT, USA) for 5 min. The supernatants were collected and analyzed by LC-MS/MS to determine the in vitro plasma half-life (t_{1/2}).

Liver S9 stability assay. The in vitro liver S9 stability assay was conducted in duplicate in commercially available mouse and human liver S9 fractions (Sekisui XenoTech; Kansas City, KS, USA), which were supplemented with either nicotinamide adenine dinucleotide phosphate (NADPH) or uridine 5′-diphosphoglucuronic acid (UDPGA) as a cofactor. Briefly, a compound (1 mol/L final concentration) was spiked into the reaction mixture containing liver microsomal protein (1 mg/mL final concentration), MgCl₂ (1 mmol/L final concentration), and/or alamethicin (50 μg/mg protein, only in incubation with cofactor UDPGA) in 0.1 mol/L potassium phosphate buffer (pH 7.4). For phase I metabolism, the reaction was initiated by addition of 1 mmol/L NADPH, followed by incubation at 37° C. A negative control was performed in parallel without NADPH to reveal any chemical instability or non-NADPH dependent enzymatic degradation for each compound. A reaction with positive control verapamil was also performed as an in-house quality control to confirm the proper functionality of the incubation systems. For phase II metabolic conjugation, the mixture was first pre-incubated on ice for 10 min to allow the pore-formation peptide alamethicin to penetrate the membrane of microsomes and to expose UGT enzymes. The reaction was then initiated by the addition of 5 mmol/L UDPGA, followed by incubation at 37° C. A negative control was performed in parallel without UDPGA to reveal any chemical instability or non-UDPGA dependent enzymatic degradation for each compound. A reaction with positive control umbelliferone was also performed as an in-house quality control to confirm the proper functionality of the incubation systems. At various time points (0, 5, 15, 30, 45 or 60 min), a 50 μL aliquot of reaction was taken and quenched with 150 μL of acetonitrile containing 0.1% formic acid. The samples were then vortexed and centrifuged at 15,000 rpm for 5 min at 4° C. The supernatants were collected and analyzed by LC-MS/MS to determine the in vitro metabolic half-life (t_{1/2}).

Parallel artificial membrane permeability assay (PAMPA).⁵ The membrane permeability of selected compounds was evaluated using the Corning® BioCoat™ Pre-coated PAMPA Plate System (Corning 353015; Glendale, AZ, USA). The pre-coated plate assembly, which was stored at −20° C., was taken to thaw for 30 min at room temperature. The permeability assay was carried out in accordance with the manufacturer's protocol. Briefly, the 96-well filter plate, pre-coated with lipids, was used as the permeation acceptor and a matching 96 well receiver plate was used as the permeation donor. Compound solutions were prepared by diluting the 10 mmol/L DMSO stock solutions with 10% methanol in DPBS to a final concentration of 10 mol/L. The compound solutions were added to the wells (300 μL/well) of the receiver plate and DPBS with 10% methanol was added to the wells (200 μL/well) of the pre-coated filter plate. The filter plate was then coupled with the receiver plate and the plate assembly was incubated at 25° C. without agitation for 5 h. At the end of the incubation, the plate was separated and the final concentrations of compounds in both donor wells and acceptor wells were analyzed using LC-MS/MS. Permeability of a compound was calculated using the Eq. (1):

$$Pe = \{ - ln [ 1 - {C}_{A} ( t ) / {C}_{eq} ] \} / [ A \times ( 1 / {V}_{D} + 1 / {V}_{A} ) \times t ] \qquad ( 1 )$$

where A=filter area (0.3 cm²), V_{D}=donor well volume (0.3 mL), V_{A}=acceptor well volume (0.2 mL), t=incubation time (seconds), C_{A}(t)=compound concentration in acceptor well at time t, C_{D}(t)=compound concentration in donor well at time t, and C_{eq} was calculated using the Eq. (2):

$${C}_{eq} = [ {C}_{D} ( t ) \times {V}_{D} + {C}_{A} ( t ) \times {V}_{A} ] / ( {V}_{D} + {V}_{A} ) \qquad ( 2 )$$

A cutoff criterion of Pe value at 1.5×10⁶ cm/s was used to classify the compounds into high and low permeability according to the literature report of this PAMPA plate system¹.

### Molecular Modeling

The docking study was carried out using the Schrodinger modeling package (release 2019-3). The cryo-EM structure of SARS-CoV-2 Nsp14 (PDB 7EGQ)⁶ was taken from the Protein Data Bank. The missing residues were added to the Nsp14 structure using Prime and the energy minimization was performed using OPLS3e forcefield. Nsp14 MTase inhibitors were generated by LigPrep and docked into the Glide grid encompassing the SAM and RNA substrate binding pockets of the SARS2-CoV-2 Nsp14 MTase active site. Docking was carried out in the XP mode with no constraints. Structural visualization and representation were performed using PyMOL.

### REFERENCES

1. Aouadi, W.; Eydoux, C.; Coutard, B.; Martin, B.; Debart, F.; Vasseur, J. J.; Contreras, J. M.; Morice, C.; Querat, G.; Jung, M. L.; Canard, B.; Guillemot, J. C.; Decroly, E., Toward the identification of viral cap-methyltransferase inhibitors by fluorescence screening assay. Antiviral Res. 2017, 144, 330-339.
2. Chen, D. Y.; Khan, N.; Close, B. J.; Goel, R. K.; Blum, B.; Tavares, A. H.; Kenney, D.; Conway, H. L.; Ewoldt, J. K.; Chitalia, V. C.; Crossland, N. A.; Chen, C. S.; Kotton, D. N.; Baker, S. C.; Fuchs, S. Y.; Connor, J. H.; Douam, F.; Emili, A.; Saeed, M., SARS-CoV-2 Disrupts Proximal Elements in the JAK-STAT Pathway. J. Virol. 2021, 95 (19), e0086221.
3. Soto-Acosta, R.; Edwards, T. C.; Dreis, C. D.; Krishna, V. D.; Cheeran, M. C.; Qiu, L.; Xie, J.; Bonnac, L. F.; Geraghty, R. J., Enhancing the Antiviral Potency of Nucleobases for Potential Broad-Spectrum Antiviral Therapies. Viruses 2021, 13 (12).
4. Soto-Acosta, R.; Jung, E.; Qiu, L.; Wilson, D. J.; Geraghty, R. J.; Chen, L., 4,7-Disubstituted 7H-Pyrrolo[2,3-d]pyrimidines and Their Analogs as Antiviral Agents against Zika Virus. Molecules 2021, 26 (13), 3779.
5. Chen, X.; Murawski, A.; Patel, K.; Crespi, C. L.; Balimane, P. V., A novel design of artificial membrane for improving the PAMPA model. Pharm. Res. 2008, 25 (7), 1511-20.
6. Yan, L.; Yang, Y.; Li, M.; Zhang, Y.; Zheng, L.; Ge, J.; Huang, Y. C.; Liu, Z.; Wang, T.; Gao, S.; Zhang, R.; Huang, Y. Y.; Guddat, L. W.; Gao, Y.; Rao, Z.; Lou, Z., Coupling of N7-methyltransferase and 3′-5′ exoribonuclease with SARS-CoV-2 polymerase reveals mechanisms for capping and proofreading. Cell 2021, 184 (13), 3474-3485 e11.
7. Gao, Y.; van Haren, M. J.; Moret, E. E.; Rood, J. J. M.; Sartini, D.; Salvucci, A.; Emanuelli, M.; Craveur, P.; Babault, N.; Jin, J.; Martin, N. I., Bisubstrate Inhibitors of Nicotinamide N-Methyltransferase (NNMT) with Enhanced Activity. J. Med. Chen. 2019, 62 (14), 6597-6614.
8. Liu, F.; Austin, D. J., Synthesis of 5′-functionalized adenosine: suppression of cyclonucleoside formation. Tetrahedron Lett. 2001, 42 (18), 3153-3154.
9. Wintner, E. A.; Conn, M. M.; Rebek, J., Self-Replicating Molecules—a 2nd Generation. J. Am. Chem. Soc. 1994, 116 (20), 8877-8884.
10. Rowan, A. S.; Nicely, N. I.; Cochrane, N.; Wlassoff, W. A.; Claiborne, A.; Hamilton, C. J., Nucleoside triphosphate mimicry: a sugar triazolyl nucleoside as an ATP-competitive inhibitor of B. anthracis pantothenate kinase. Org Biomol Chem 2009, 7 (19), 4029-4036.
11. Metobo, S. E.; Xu, J.; Saunders, O. L.; Butler, T.; Aktoudianakis, E.; Cho, A.; Kim, C. U., Practical synthesis of 1′-substituted Tubercidin C-nucleoside analogs. Tetrahedron Lett. 2012, 53 (5), 484-486.
12. Li, Q.; Lescrinier, E.; Groaz, E.; Persoons, L.; Daelemans, D.; Herdewijn, P.; De Jonghe, S., Synthesis and Biological Evaluation of Pyrrolo[2,1-f][1,2,4]triazine C-Nucleosides with a Ribose, 2′-Deoxyribose, and 2′,3′-Dideoxyribose Sugar Moiety. ChemMedChem 2018, 13 (1), 97-104.

### Synthesis and Characterization

General Procedures. All commercial reagents were used as provided unless otherwise indicated. An anhydrous solvent dispensing system (J. C. Meyer) using 2 packed columns of neutral alumina was used for drying THF, Et₂O, and CH₂Cl₂, whereas 2 packed columns of molecular sieves were used to dry DMF. Solvents were dispensed under argon. Flash chromatography was performed with RediSep Rr silica gel columns on a Teledyne ISCO CombiFlash® R_{f} system using the solvents as indicated. Nuclear magnetic resonance spectra were recorded on a Varian 600 MHz or Bruker 400 MHz spectrometer with Me₄Si or signals from residual solvent as the internal standard for ¹H or ¹³C. Chemical shifts are reported in ppm, and signals are described as s (singlet), d (doublet), t (triplet), q (quartet), m (multiplet), br s (broad singlet), and dd (double doublet). Values given for coupling constants are first order. High resolution mass spectra were recorded on an Agilent TOF II TOF/MS instrument equipped with either an ESI or APCI interface.

The synthesis of adenosine derivatives began with commercially available 2′,3′-O-isopropylideneadenosine (12), which was converted into the corresponding azide 13, after treatment with diphenylphosphoryl azide (DPPA) and subsequent displacement with sodium azide (Scheme 1). Reduction of azide 13 gave primary amine 14, which was converted into secondary amine 15, amide 16, and sulfonamides 17-19 under conventional conditions. Subsequent deprotection of the acetonide group gave compounds 1-5 in 44-92% yield. To selectively obtain N6-acetylated adenosine derivative 6, protected azide 13 was treated with acetic anhydride to give acetamide 20 in 67% yield. Reduction of azide 20 gave primary amine 21, which was then coupled with 2-naphthalenesulfonyl chloride to give sulfonamide 22. Subsequent deprotection of the acetonide group gave final product N6-acetylated derivative 6.

To mitigate the high polarity associated with adenosine-derived sulfonamides, di-isobutyrate and diacetate prodrugs 7 and 8 were synthesized, which was prepared by direct treatment of sulfonamide 3 with isobutyric anhydride and acetic anhydride, respectively (Scheme 1). Similarly, triacetate 9 was prepared, a diacetate prodrug of 6-acetamide 6, whose synthesis started with common intermediate azide 13. Deprotection followed by treatment with acetic anhydride at 50° C. gave triacetate 24 in 56% yield after a tetra-acetate byproduct was removed by column chromatography. Reduction of the azide functionality in 24 was accomplished by hydrogenation in dioxane, a solvent that was chosen to mitigate undesired deacetylation in methanol. The resulting primary amine 25 was treated with 2-naphthalenesulfonyl chloride to give triacetate derivative 9. The positions of acetylation were confirmed by the heteronuclear multiple bond correlation (HMBC) NIR experiments.

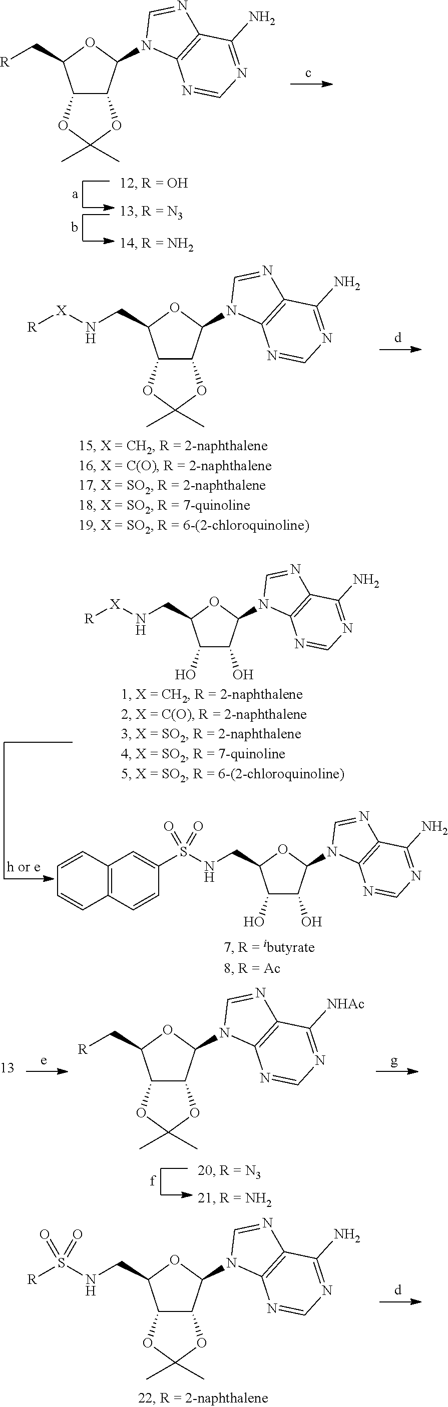

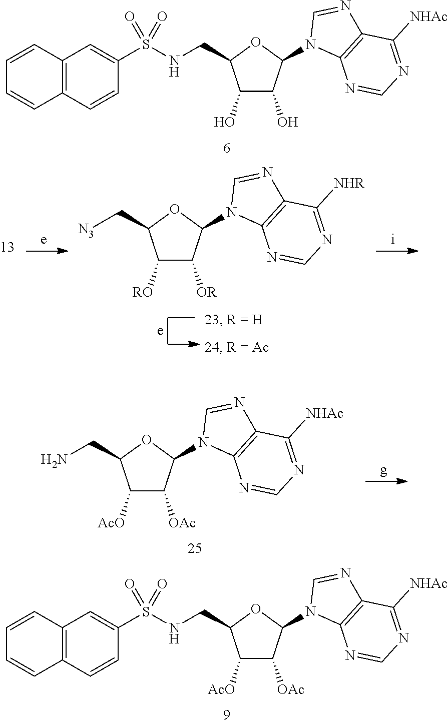

Scheme 1. Reagents and conditions: (a) i. DPPA, DBU, dioxane; ii. NaN₃, TBAI, 15-crown-5, dioxane, reflux; (b) H₂, Pd/C, MeOH; (c) for 15, 2-naphthaldehyde, NaBH₄, MeOH, 5 h, 88%; for 16, 2-naphthoyl chloride, NEt₃, CH₂Cl₂, rt, 18 h, 93%; for 17-19, sulfonyl chloride, NEt₃, CH₂Cl₂, rt, 18 h, 33-93%; (d) TFA/H₂O (4:1), rt, 3 h, 38-92%; (e) Ac₂O, pyridine, rt, 52-67%; (f) H₂, Pd/C, EtOH, 18 h, 69%; (g) 2-naphthalenesulfonyl chloride, NEt₃, CH₂Cl₂, rt, 18 h, 41-44%; (h) isobutyric anhydride, pyridine, rt, 2 h, 29%; (i) H₂, Pd/C, dioxane, 18 h.

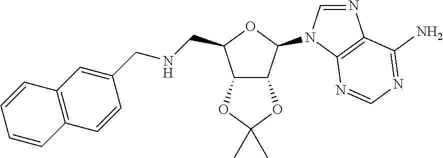 [SMILES: CC1(C)O[C@@H]2[C@H](O1)[C@@H](CNCc1ccc3ccccc3c1)O[C@H]2n1cnc2c(N)ncnc21]

9-((3aR,4R,6R,6aR)-2,2-Dimethyl-6-(((naphthalen-2-ylmethyl)amino)methyl)tetrahydrofuro[3,4-d][1,3]dioxol-4-yl)-9H-purin-6-amine (15).⁷ To a solution of compound 14⁸ (170 mg, 0.55 mmol, 1.0 eq) in methanol (20 mL) at room temperature under argon atmosphere was added 2-naphthaldehyde (130 mg, 0.83 mmol, 1.5 eq). After 3 h, NaBH₄ (84 mg, 2.22 mmol, 4.0 eq) was added, and the reaction mixture was stirred for 2 h at room temperature. Water (20 mL) was added, the aqueous layer was extracted with ethyl acetate and the combined organic extracts were washed with brine, dried over Na₂SO₄. The solvent was removed under vacuum and the crude product was purified by column chromatography (10% MeOH in CH₂Cl₂) to give compound 15 as a white solid (210 mg, 88%). ¹H NMR (400 MHz, CDCl₃) δ 8.13 (s, 1H), 7.88 (s, 1H), 7.84-7.76 (m, 3H), 7.71 (s, 1H), 7.48-7.39 (m, 4H), 5.99 (d, J=3.3 Hz, 1H), 5.82 (s, 2H), 5.48 (dd, J=6.4, 3.3 Hz, 1H), 5.07 (dd, J=6.5, 3.2 Hz, 1H), 4.45-4.40 (m, 1H), 3.96 (s, 2H), 2.98 (dd, J=12.8, 4.2 Hz, 1H), 2.91 (dd, J=12.6, 6.0 Hz, 1H), 1.61 (s, 3H), 1.38 (s, 3H). HRMS (ESF): m/z calcd for C₂₄H₂₇N₆O₃ [M+H]⁺447.2139, found 447.2128.

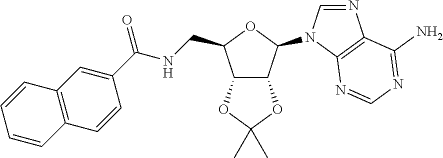 [SMILES: CC1(C)O[C@@H]2[C@H](O1)[C@@H](CNC(=O)c1ccc3ccccc3c1)O[C@H]2n1cnc2c(N)ncnc21]

N-(((3aR,4R,6R,6aR)-6-(6-Amino-9H-purin-9-yl)-2,2-dimethyltetrahydrofuro[3,4-d][1,3]dioxol-4-yl)methyl)-2-naphthamide (16).⁹ To a solution of compound 14⁸ (95 mg, 0.31 mmol, 1.0 eq) in CH₂Cl₂ (20 mL) at room temperature under argon atmosphere was added triethylamine (0.13 mL, 0.93 mmol, 3.0 eq). The reaction mixture was cooled to 0° C. and 2-naphthoyl chloride (88 mg, 0.47 mmol, 1.5 eq) was added. The reaction mixture was stirred for 18 h at room temperature. Water (10 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 16 as a white solid (135 mg, 93%). ¹H NMR (600 MHz, CDCl₃) δ 8.38 (s, 1H), 8.28 (dd, J=8.4, 2.6 Hz, 1H), 7.96-7.86 (m, 5H), 7.82 (s, 1H), 7.58-7.52 (m, 2H), 5.92 (s, 2H), 5.88 (d, J=4.4 Hz, 1H), 5.40 (dd, J=6.3, 4.4 Hz, 1H), 5.05 (dd, J=6.4, 2.7 Hz, 1H), 4.60 (d, J=3.1 Hz, 1H), 4.38 (ddd, J=14.5, 8.3, 3.4 Hz, 1H), 3.60 (dt, J=14.5, 2.7 Hz, 1H), 1.64 (s, 3H), 1.37 (s, 3H). HRMS (ESI): m/z calcd for C₂₄H₂₅N₆O₄ [M+H]⁺ 461.1932, found 461.1928.

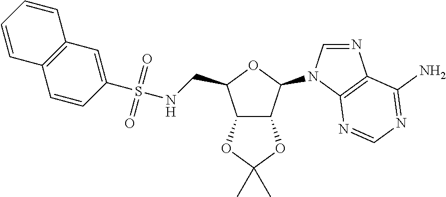 [SMILES: CC1(C)O[C@@H]2[C@H](O1)[C@@H](CNS(=O)(=O)c1ccc3ccccc3c1)O[C@H]2n1cnc2c(N)ncnc21]

N-(((3aR,4R,6R,6aR)-6-(6-Amino-9H-purin-9-yl)-2,2-dimethyltetrahydrofuro[3,4-d][1,3]dioxol-4-yl)methyl)naphthalene-2-sulfonamide (17). To a solution of compound 14⁸ (94 mg, 0.31 mmol, 1.0 eq) in CH₂Cl₂ (20 mL) at room temperature under argon atmosphere was added triethylamine (0.13 mL, 0.92 mmol, 3.0 eq). The reaction mixture was cooled to 0° C. and 2-naphthalenesulfonyl chloride (110 mg, 0.46 mmol, 1.5 eq) was added. The reaction mixture was stirred for 18 h at room temperature. Water (10 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 17 as a white solid (130 mg, 93%). 1H NMR (600 MHz, CDCl₃) δ 9.31 (d, J=10.0 Hz, 1H), 8.66 (s, 1H), 8.42 (s, 1H), 8.01-7.85 (m, 3H), 7.80-7.77 (m, 2H), 7.67-7.56 (m, 2H), 5.74 (d, J=5.1 Hz, 1H), 5.64 (s, 2H), 5.28 (t, J=5.7 Hz, 1H), 4.95 (dd, J=6.2, 1.7 Hz, 1H), 4.50 (d, J=1.7 Hz, 1H), 3.56-3.48 (m, 1H), 3.18 (dd, J=13.0, 2.5 Hz, 1H), 1.59 (s, 3H), 1.25 (s, 3H). HRMS (ESI⁺): m/z calcd for C₂₃H₂₅N₆O₅S [M+H]⁺ 497.1607, found 497.1601.

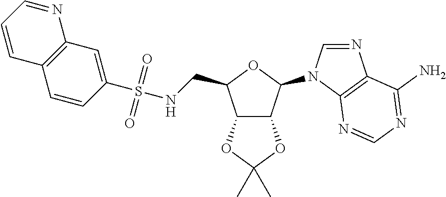 [SMILES: CC1(C)O[C@@H]2[C@H](O1)[C@@H](CNS(=O)(=O)c1ccc3cccnc3c1)O[C@H]2n1cnc2c(N)ncnc21]

N-(((3aR,4R,6R,6aR)-6-(6-Amino-9H-purin-9-yl)-2,2-dimethyltetrahydrofuro[3,4-d][1,3]dioxol-4-yl)methyl)quinoline-7-sulfonamide (18). To a solution of compound 14 (84 mg, 0.27 mmol, 1.0 eq.) in CH₂Cl₂ (20 mL) at room temperature under argon atmosphere was added triethylamine (0.12 mL, 0.82 mmol, 3.0 eq.). The mixture was cooled to 0° C. and quinoline-7-sulfonyl chloride (93 mg, 0.41 mmol, 1.5 eq.) was added. The reaction mixture was stirred for 18 h at room temperature. Water (10 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 18 as a white solid (41 mg, 33%). ¹H NMR (600 MHz, CDCl₃) δ 9.53 (d, J=10.0 Hz, 1H), 9.03 (dd, J=4.1, 1.7 Hz, 1H), 8.64 (s, 1H), 8.63 (s, 1H), 8.19 (dd, J=8.2, 1.7 Hz, 1H), 7.92 (s, 2H), 7.80 (s, 1H), 7.52 (dd, J=8.3, 4.2 Hz, 1H), 5.96 (s, 2H), 5.76 (d, J=5.0 Hz, 1H), 5.29 (d, J=6.2 Hz, 1H), 5.00 (dd, J=6.1, 1.8 Hz, 1H), 4.51 (d, J=2.1 Hz, 1H), 3.58 (ddd, J=12.7, 10.1, 2.2 Hz, 1H), 3.20 (dd, J=12.9, 2.5 Hz, 1H), 1.59 (s, 3H), 1.28 (s, 3H). HRMS (ESI⁺): m/z calcd for C₂₂H₂₄N₇O₅S [M+H]⁺ 498.1554, found 498.1552.

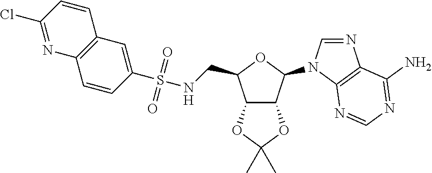 [SMILES: CC1(C)O[C@@H]2[C@H](O1)[C@@H](CNS(=O)(=O)c1ccc3nc(Cl)ccc3c1)O[C@H]2n1cnc2c(N)ncnc21]

N-(((3aR,4R,6R,6aR)-6-(6-Amino-9H-purin-9-yl)-2,2-dimethyltetrahydrofuro[3,4-d][1,3]dioxol-4-yl)methyl)-2-chloroquinoline-6-sulfonamide (19). To a solution of compound 14 (107 mg, 0.35 mmol, 1.0 eq.) in CH₂Cl₂ (20 mL) at room temperature under argon atmosphere was added triethylamine (0.15 mL, 1.05 mmol, 3.0 eq.). The mixture was cooled to 0° C. and 2-chloroquinoline-6-sulfonyl chloride (109 mg, 0.42 mmol, 1.2 eq.) was added. The reaction mixture was stirred for 18 h at room temperature. Water (10 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 19 as a white solid (105 mg, 54%). ¹H NMR (600 MHz, CDCl₃) δ 9.51 (d, J=9.9 Hz, 1H), 8.62 (s, 1H), 8.40 (d, J=2.0 Hz, 1H), 8.19 (d, J=8.6 Hz, 1H), 8.09 (d, J=8.9 Hz, 1H), 8.04 (dd, J=8.9, 2.0 Hz, 1H), 7.80 (s, 1H), 7.50 (d, J=8.6 Hz, 1H), 5.77 (d, J=5.0 Hz, 1H), 5.73 (s, 2H), 5.27 (t, J=5.6 Hz, 1H), 5.00 (dd, J=6.4, 1.9 Hz, 1H), 4.51 (d, J=2.2 Hz, 1H), 3.54 (ddd, J=12.6, 9.9, 2.2 Hz, 1H), 3.19 (dd, J=13.1, 2.5 Hz, 1H), 1.60 (s, 3H), 1.30 (s, 3H). HRMS (ESI⁺): m/z calcd for C₂₂H₂₃C₁N205S [M+H]⁺ 532.1164, found 532.1161.

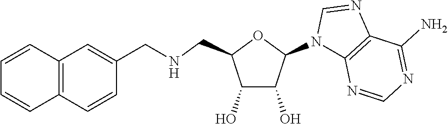 [SMILES: Nc1ncnc2c1ncn2[C@@H]1O[C@H](CNCc2ccc3ccccc3c2)[C@@H](O)[C@H]1O]

(2R,3R,4S,5R)-2-(6-Amino-9H-purin-9-yl)-5-(((naphthalen-2-ylmethyl)amino)methyl)tetrahydrofuran-3,4-diol (1). To compound 15 (104 mg, 0.22 mmol) was added a solution of TFA/H₂O (5 mL, 4:1) at 0° C. The reaction mixture was stirred at 0° C. for 30 min, allowed to warm to room temperature and stirred for a further 3 h. The reaction mixture was diluted with methanol (10 mL) and the solvents were removed under vacuum. The crude product was purified by column chromatography (20% MeOH in CH₂Cl₂) to give compound 1 as a white solid (90 mg, 67%). ¹H NMR (400 MHz, CD₃OD) δ 8.21 (s, 1H), 7.96 (s, 1H), 7.84-7.77 (m, 4H), 7.49-7.45 (m, 3H), 5.97 (d, J=5.6 Hz, 1H), 4.84-4.82 (m, 1H), 4.36 (t, J=4.7 Hz, 1H), 4.30 (dd, J=7.4, 3.6 Hz, 1H), 4.12 (d, J=15.2 Hz, 2H), 3.19 (dd, J=12.8, 7.4 Hz, 1H), 3.13 (dd, J=12.8, 3.6 Hz, 1H). ¹³C NMR (100 MHz, CD₃OD) δ 157.4, 153.8, 150.4, 142.1, 135.5, 134.8, 134.4, 129.4, 128.9, 128.8, 128.7, 127.6, 127.4, 127.2, 121.2, 91.2, 84.2, 74.5, 73.3, 54.0, 51.2. HRMS (ESI⁺): m/z calcd for C₂₁H₂₃N₆O₃ [M+H]⁺ 407.1826, found 407.1816.

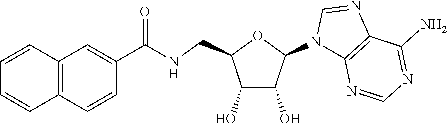 [SMILES: Nc1ncnc2c1ncn2[C@@H]1O[C@H](CNC(=O)c2ccc3ccccc3c2)[C@@H](O)[C@H]1O]

N-(((2R,3S,4R,5R)-5-(6-Amino-9H-purin-9-yl)-3,4-dihydroxytetrahydrofuran-2-yl)methyl)-2-naphthamide (2). To compound 16 (135 mg, 0.29 mmol) was added a solution of TFA/H₂O (6.25 mL, 4:1) at 0° C. The reaction mixture was stirred at 0° C. for 30 min, allowed to warm to room temperature and stirred for a further 3 h. The reaction mixture was diluted with methanol (10 mL) and the solvents were removed under vacuum. The crude product was purified by column chromatography (15% MeOH in CH₂Cl₂) to give compound 2 as a white solid (95 mg, 79%). ¹H NMR (400 MHz, DMSO-d₆) δ 8.89 (t, J=5.8 Hz, 1H), 8.46 (d, J=1.6 Hz, 1H), 8.38 (s, 1H), 8.07 (s, 1H), 8.02-7.93 (m, 4H), 7.63-7.57 (m, 2H), 7.35 (s, 2H), 5.89 (d, J=6.2 Hz, 1H), 5.47 (s, 1H), 5.30 (s, 1H), 4.78 (t, J=5.7 Hz, 1H), 4.23 (dd, J=5.1, 3.2 Hz, 1H), 4.15-4.13 (m, 1H), 3.68 (t, J=5.8 Hz, 2H). BC NMR (100 MHz, DMSO-d₆) δ 166.8, 156.0, 152.3, 149.3, 140.4, 134.1, 132.1, 131.8, 128.8, 127.9, 127.6 (×2), 127.5, 126.7, 124.2, 119.4, 87.7, 83.3, 72.6, 71.3, 41.8. HRMS (ESI⁺): m/z calcd for C₂₁H₂₁N₆O₄ [M+H]⁺ 421.1619, found 421.1608.

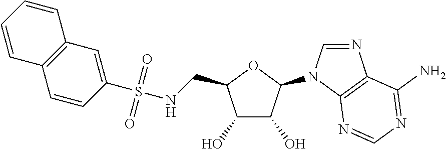 [SMILES: Nc1ncnc2c1ncn2[C@@H]1O[C@H](CNS(=O)(=O)c2ccc3ccccc3c2)[C@@H](O)[C@H]1O]

N-(((2R,3S,4R,5R)-5-(6-Amino-9H-purin-9-yl)-3,4-dihydroxytetrahydrofuran-2-yl)methyl)naphthalene-2-sulfonamide (3). To compound 17 (130 mg, 0.26 mmol) was added a solution of TFA/1120 (6.25 mL, 4:1) at 0° C. The reaction mixture was stirred at 0° C. for 30 min, allowed to warm to room temperature and stirred for a further 3 h. The reaction mixture was diluted with methanol (10 mL) and the solvents were removed under vacuum. The crude product was purified by column chromatography (15% MeOH in CH₂Cl₂) to give compound 3 as a white solid (90 mg, 75%). ¹H NMR (400 MHz, CD₃OD) δ 8.41 (d, J=1.9 Hz, 1H), 8.29 (s, 1H), 8.18 (s, 1H), 7.98-7.95 (m, 2H), 7.91 (dd, J=8.0, 1.2 Hz, 1H), 7.79 (dd, J=8.7, 1.9 Hz, 1H), 7.65-7.56 (m, 2H), 5.86 (d, J=6.7 Hz, 1H), 4.86 (d, J=5.6 Hz, 1H), 4.25 (dd, J=5.4, 2.5 Hz, 1H), 4.21 (q, J=3.2 Hz, 1H), 3.36 (dd, J=13.5, 3.6 Hz, 1H), 3.20 (dd, J=13.5, 3.3 Hz, 1H). BC NMR (100 MHz, CD₃OD) δ 157.7, 153.6, 149.9, 142.4, 138.4, 136.1, 133.5, 130.6, 130.2, 129.8, 129.1, 129.0, 128.7, 123.2, 121.2, 91.4, 85.8, 74.3, 73.0, 46.0. HRMS (ESI⁺): m/z calcd for C₂₀H₂₁N₆O₅S [M+H]⁺ 457.1289, found 457.1289.

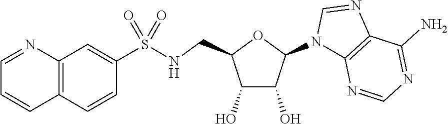 [SMILES: Nc1ncnc2c1ncn2[C@@H]1O[C@H](CNS(=O)(=O)c2ccc3cccnc3c2)[C@@H](O)[C@H]1O]

N-(((2R,3S,4R,5R)-5-(6-Amino-9H-purin-9-yl)-3,4-dihydroxytetrahydrofuran-2-yl)methyl)quinoline-7-sulfonamide (4). To compound 18 (41 mg, 0.08 mmol) was added a solution of TFA/H₂O (2.5 mL, 4:1) at 0° C. The reaction mixture was stirred at 0° C. for 30 min, allowed to warm to room temperature and stirred for a further 3 h. The reaction mixture was diluted with methanol (5 mL) and the solvents were removed under vacuum. The crude product was purified by column chromatography (20% MeOH in CH₂Cl₂) to give compound 4 as a white solid (16 mg, 440%). ¹H NMR (600 MHz, CD₃OD) δ 8.98 (dd, J=4.3, 1.6 Hz, 1H), 8.53 (t, J=1.2 Hz, 1H), 8.43 (dd, J=8.3, 1.6 Hz, 1H), 8.28 (s, 1H), 8.17 (s, 1H), 8.09 (d, J=8.6 Hz, 1H), 7.95 (dd, J=8.6, 1.8 Hz, 1H), 7.67 (dd, J=8.4, 4.3 Hz, 1H), 5.83 (d, J=6.6 Hz, 1H), 4.83 (t, J=6.2 Hz, 1H), 4.22 (dd, J=5.5, 2.7 Hz, 1H), 4.19 (q, J=3.4 Hz, 1H), 3.41 (dd, J=13.5, 3.8 Hz, 1H), 3.26 (dd, J=13.5, 3.4 Hz, 1H). ¹³C NMR (150 MHz, CD₃OD) δ 157.8, 153.7, 153.3, 147.9, 143.0, 142.3, 142.0, 138.2, 137.7, 131.6, 131.1, 128.9, 124.8, 124.5, 91.4, 85.7, 74.3, 72.9, 46.2. HRMS (ESI⁺): m/z calcd for C₁₉H₂₀N₇O₅S [M+H]⁺ 458.1241, found 458.1241.

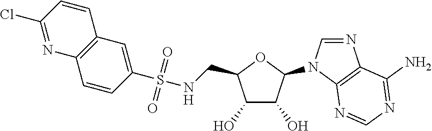 [SMILES: Nc1ncnc2c1ncn2[C@@H]1O[C@H](CNS(=O)(=O)c2ccc3nc(Cl)ccc3c2)[C@@H](O)[C@H]1O]

N-(((2R,3S,4R,5R)-5-(6-Amino-9H-purin-9-yl)-3,4-dihydroxytetrahydrofuran-2-yl)methyl)-2-chloroquinoline-6-sulfonamide (5). To compound 19 (105 mg, 0.20 mmol) was added a solution of TFA/H₂O (5 mL, 4:1) at 0° C. The reaction mixture was stirred at 0° C. for 30 min, allowed to warm to room temperature and stirred for a further 3 h. The reaction mixture was diluted with methanol (10 mL) and the solvents were removed under vacuum. The crude product was purified by column chromatography (10% MeOH in CH₂Cl₂) to give compound 5 as a white solid (45 mg, 92%). ¹H NMR (400 MHz, DMSO-d₆) δ 8.72 (t, J=5.6 Hz, 1H), 8.66 (d, J=8.6 Hz, 1H), 8.59 (d, J=1.8 Hz, 1H), 8.28 (s, 1H), 8.14-8.08 (m, 3H), 7.74 (d, J=8.6 Hz, 1H), 7.40 (s, 2H), 5.80 (d, J=6.4 Hz, 1H), 5.47 (s, 1H), 5.27 (s, 1H), 4.66 (t, J=6.4 Hz, 1H), 4.06-4.04 (m, 1H), 4.00-3.97 (m, 1H), 3.19-3.13 (m, 2H). ¹³C NMR (100 MHz, DMSO-d₆) δ 156.1, 152.4, 152.1, 148.8, 148.2, 141.2, 140.4, 138.6, 129.5, 127.8, 127.2, 126.0, 123.9, 119.5, 88.1, 83.5, 72.4, 71.1, 44.9. HRMS (ESI⁺): m/z calcd for C₁₉H₁₉ClN₇O₅S [M+H]⁺ 492.0851, found 492.0833.

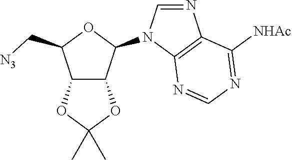 [SMILES: CC(=O)Nc1ncnc2c1ncn2[C@@H]1O[C@H](CN=[N+]=[N-])[C@H]2OC(C)(C)O[C@H]21]

N-(9-((3aR,4R,6R,6aR)-6-(Azidomethyl)-2,2-dimethyltetrahydrofuro[3,4-d][1,3]dioxol-4-yl)-9H-purin-6-yl)acetamide (20). To a solution of compound 138 (240 mg, 0.72 mmol, 1.0 eq.) in dry pyridine (10 mL) at room temperature was added acetic anhydride (0.68 mL, 7.22 mmol, 10.0 eq.). The resultant solution was stirred at room temperature for 24 h. The solvents were removed under vacuum. The residue was co-evaporated with methanol. The crude product was purified by column chromatography (95% ethyl acetate in hexane) to give compound 20 as a white solid (180 mg, 67%). ¹H NMR (600 MHz, CDCl₃) δ 9.03 (s, 1H), 8.70 (s, 1H), 8.17 (s, 1H), 6.16 (d, J=2.4 Hz, 1H), 5.44 (dd, J=6.4, 2.4 Hz, 1H), 5.05 (dd, J=6.4, 3.5 Hz, 1H), 4.40 (td, J=5.3, 3.5 Hz, 1H), 3.61-3.55 (m, 2H), 2.63 (s, 3H), 1.62 (s, 3H), 1.39 (s, 3H). HRMS (ESI⁺): m/z calcd for C₁₅H₁₉N₈O₄ [M+H]⁺ 375.1524, found 375.1519.

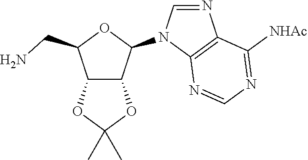 [SMILES: CC(=O)Nc1ncnc2c1ncn2[C@@H]1O[C@H](CN)[C@H]2OC(C)(C)O[C@H]21]

N-(9-((3aR,4R,6R,6aR)-6-(Aminomethyl)-2,2-dimethyltetrahydrofuro[3,4-d][1,3]dioxol-4-yl)-9H-purin-6-yl)acetamide (21). To a solution of compound 20 (180 mg, 0.48 mmol) in ethanol (5 mL) was added 10% Pd/C (36 mg, 20% w/w) and the mixture stirred under an atmosphere of hydrogen for 18 h. The mixture was filtered through Celite, washed with methanol and the solvent removed under vacuum. The crude product was purified by column chromatography (35% MeOH in CH₂Cl₂) to give compound 21 as a white solid (110 mg, 69%). 1H NMR (400 MHz, CD₃OD) δ 8.62 (s, 1H), 8.50 (s, 1H), 6.27 (d, J=2.8 Hz, 1H), 5.50 (dd, J=6.4, 2.7 Hz, 1H), 5.09 (dd, J=6.4, 3.4 Hz, 1H), 4.34 (ddd, J=7.4, 5.0, 3.5 Hz, 1H), 3.19-3.00 (m, 2H), 2.36 (s, 3H), 1.61 (s, 3H), 1.39 (s, 3H). HRMS (ESI^{T}): m/z calcd for C₁₅H₂₁N₆O₄ [M+H]⁺ 349.1619, found 349.1619.

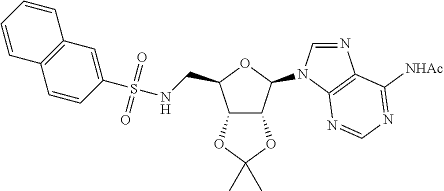 [SMILES: CC(=O)Nc1ncnc2c1ncn2[C@@H]1O[C@H](CNS(=O)(=O)c2ccc3ccccc3c2)[C@H]2OC(C)(C)O[C@H]21]

N-(9-((3aR,4R,6R,6aR)-2,2-Dimethyl-6-((naphthalene-2-sulfonamido)methyl)tetrahydrofuro[3,4-d][1,3]dioxol-4-yl)-9H-purin-6-yl)acetamide (22). To compound 21 (110 mg, 0.32 mmol, 1.0 eq.) in CH₂Cl₂ (25 mL) under argon atmosphere at room temperature was added triethylamine (0.13 mL, 0.95 mmol, 3.0 eq.). The mixture was cooled to 0° C. and 2-naphthalenesulfonyl chloride (107 mg, 0.47 mmol, 1.5 eq.) was added. The reaction mixture was stirred for 24 h at room temperature. Water (20 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 22 as a white solid (74 mg, 44%). ¹H NMR (600 MHz, CDCl₃) δ 9.34 (s, 1H), 8.96 (s, 1H), 8.41 (d, J=1.8 Hz, 1H), 8.10 (s, 1H), 7.97-7.83 (m, 3H), 7.77 (dd, J=8.6, 1.9 Hz, 1H), 7.65-7.51 (m, 2H), 5.83 (d, J=4.7 Hz, 1H), 5.26 (dd, J=6.2, 4.8 Hz, 1H), 4.95 (dd, J=6.3, 2.1 Hz, 1H), 4.47 (d, J=2.4 Hz, 1H), 3.51 (dd, J=12.8, 2.8 Hz, 1H), 3.19 (dd, J=12.8, 2.2 Hz, 1H), 2.66 (s, 3H), 1.57 (s, 4H), 1.24 (s, 3H). HRMS (ESI⁺): m/z calcd for C₂₅H₂₇N₆O₆S [M+H]⁺ 539.1707, found 539.1711.

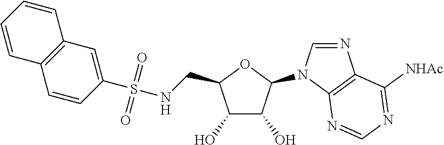 [SMILES: CC(=O)Nc1ncnc2c1ncn2[C@@H]1O[C@H](CNS(=O)(=O)c2ccc3ccccc3c2)[C@@H](O)[C@H]1O]

N-(9-((2R,3R,4S,5R)-3,4-Dihydroxy-5-((naphthalene-2-sulfonamido)methyl)tetrahydrofuran-2-yl)-9H-purin-6-yl)acetamide (6). To compound 22 (70 mg, 0.13 mmol) was added a solution of TFA/H₂O (3.75 mL, 4:1) at 0° C. The reaction mixture was stirred at 0° C. for 30 min, allowed to warm to room temperature and stirred for a further 3 h.

The reaction mixture was diluted with methanol (10 mL) and the solvents were removed under vacuum. The crude product was purified by column chromatography (10% MeOH in CH₂Cl₂) to give compound 6 as a white solid (24 mg, 38%). ¹H NMR (400 MHz, CD₃OD) δ 8.65 (s, 1H), 8.43 (s, 1H), 8.42 (d, J=2.1 Hz, 1H), 8.03-7.97 (m, 2H), 7.94 (d, J=8.1 Hz, 1H), 7.82 (dd, J=8.7, 2.0 Hz, 1H), 7.67-7.59 (m, 2H), 5.94 (d, J=6.3 Hz, 1H), 4.86 (t, J=5.8 Hz, 1H), 4.29 (dd, J=5.4, 3.1 Hz, 1H), 4.18 (q, J=4.1 Hz, 1H), 3.36 (dd, J=13.7, 4.1 Hz, 1H), 3.26 (dd, J=13.7, 3.7 Hz, 1H), 2.39 (s, 3H). ¹³C NMR (100 MHz, CD₃OD) δ 171.9, 153.7, 152.8, 149.9, 145.1, 142.3, 138.6, 136.2, 133.6, 130.6, 130.2, 129.9, 129.1, 129.0, 128.7, 123.2, 91.4, 85.6, 74.2, 73.0, 46.1, 24.8. HRMS (ESI⁺): m/z calcd for C₂₂H₂₃N₆O₆S [M+H]⁺ 499.1394, found 499.1386.

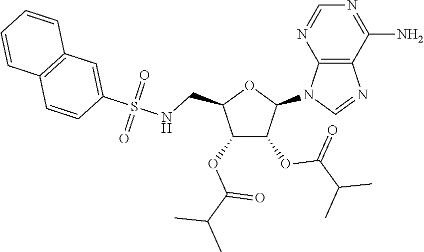 [SMILES: CC(C)C(=O)O[C@@H]1[C@H](OC(=O)C(C)C)[C@@H](CNS(=O)(=O)c2ccc3ccccc3c2)O[C@H]1n1cnc2c(N)ncnc21]

(2R,3R,4R,5R)-2-(6-Amino-9H-purin-9-yl)-5-((naphthalene-2-sulfonamido)methyl)tetrahydrofuran-3,4-diyl bis(2-methylpropanoate) (7). To a solution of compound 3 (120 mg, 0.26 mmol, 1.0 eq.) in dry pyridine (10 mL) at room temperature was added isobutyric anhydride (0.44 mL, 2.63 mmol, 10.0 eq.). The resultant solution was stirred at room temperature for 2 h. The mixture was diluted with MeOH (20 mL) and the solvents were removed under vacuum. The crude product was purified by column chromatography (95-100% ethyl acetate in hexane) to give compound 7 as a white solid (43 mg, 29%). ¹H NMR (400 MHz, DMSO-d₆) δ 8.62 (t, J=6.2 Hz, 1H), 8.46 (d, J=1.8 Hz, 1H), 8.33 (s, 1H), 8.17-8.08 (m, 3H), 8.03 (d, J=8.0 Hz, 1H), 7.81 (dd, J=8.7, 1.9 Hz, 1H), 7.73-7.65 (m, 2H), 7.44 (s, 2H), 6.14 (d, J=6.3 Hz, 1H), 5.93 (t, J=6.0 Hz, 1H), 5.49 (dd, J=5.7, 3.5 Hz, 1H), 4.23 (q, J=4.4 Hz, 1H), 3.26 (s, 2H), 2.57 (s, J=7.0 Hz, 1H), 2.44 (s, J=7.0 Hz, 1H), 1.11 (t, J=6.8 Hz, 6H), 0.98 (dd, J=11.4, 7.0 Hz, 6H). ¹³C NMR (100 MHz, DMSO-d₆) δ 174.9, 174.6, 156.3, 152.5, 148.6, 140.1, 137.1, 134.2, 131.7, 129.5, 129.2, 128.8, 127.8, 127.6, 127.5, 122.0, 119.4, 86.0, 81.2, 71.6, 71.1, 44.2, 33.0, 32.9, 18.6, 18.5, 18.4 (×2). HRMS (ESI⁺): m/z calcd for C₂₈H₃₃N₆O₇S [M+H]⁺ 597.2126, found 597.2126.

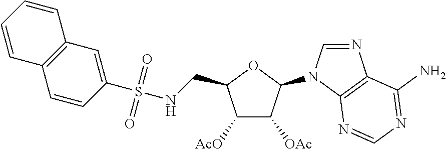 [SMILES: CC(=O)O[C@@H]1[C@H](OC(C)=O)[C@@H](CNS(=O)(=O)c2ccc3ccccc3c2)O[C@H]1n1cnc2c(N)ncnc21]

(2R,3R,4R,5R)-2-(6-Amino-9H-purin-9-yl)-5-((naphthalene-2-sulfonamido)methyl)tetrahydrofuran-3,4-diyl diacetate (8). To a solution of compound 3 (46 mg, 0.10 mmol, 1.0 eq.) in dry pyridine (4 mL) at room temperature was added acetic anhydride (0.1 mL, 0.60 mmol, 6.0 eq.). The resultant solution was stirred at room temperature for 2 h. The mixture was diluted with methanol (10 mL) and the solvents were removed under vacuum. The crude product was purified by column chromatography (95-100% ethyl acetate in hexane) to give compound 8 as a white solid (28 mg, 52%). ¹H NMR (400 MHz, DMSO-d₆) δ 8.60 (s, 1H), 8.46 (d, J=1.9 Hz, 1H), 8.34 (s, 1H), 8.17-8.08 (m, 3H), 8.03 (d, J=8.0 Hz, 1H), 7.80 (dd, J=8.7, 1.9 Hz, 1H), 7.74-7.64 (m, 2H), 7.45 (s, 2H), 6.16 (d, J=6.5 Hz, 1H), 5.96 (t, J=6.2 Hz, 1H), 5.46 (dd, J=5.8, 3.4 Hz, 1H), 4.24 (q, J=4.7 Hz, 1H), 3.27-3.16 (m, 2H), 2.06 (s, 3H), 1.98 (s, 3H). ¹³C NMR (100 MHz, DMSO-d₆) δ 169.4, 169.1, 156.3, 152.5, 148.7, 140.2, 137.1, 134.2, 131.0, 129.5, 129.2, 128.8, 127.8, 127.6, 127.5, 122.1, 119.4, 85.7, 81.0, 71.4, 71.2, 44.2, 20.3, 20.1. HRMS (ESI⁺): m/z calcd for C₂₄H₂₅N₆O₇S [M+H]⁺ 541.1500, found 541.1498.

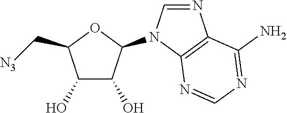 [SMILES: [N-]=[N+]=NC[C@H]1O[C@@H](n2cnc3c(N)ncnc32)[C@H](O)[C@@H]1O]

(2R,3R,4S,5R)-2-(6-Amino-9H-purin-9-yl)-5-(aminomethyl)tetrahydrofuran-3,4-diol (23). To compound 13 (260 mg, 0.78 mmol) was added a solution of TFA/H₂O (10 mL, 4:1) at 0° C. The reaction mixture was stirred at 0° C. for 30 min, allowed to warm to room temperature and stirred for a further 4 h. The reaction mixture was diluted with methanol (20 mL) and the solvents were removed under vacuum. The crude product was purified by column chromatography (20% MeOH in CH₂Cl₂) to give compound 23 as a yellow oil (190 mg, 86%). ¹H NMR (600 MHz, CD₃OD) δ 8.29 (s, 1H), 8.22 (s, 1H), 6.03 (d, J=4.7 Hz, 1H), 4.79 (t, J=5.0 Hz, 1H), 4.38 (t, J=5.1 Hz, 1H), 4.19 (d, J=4.5 Hz, 1H), 3.73-3.61 (m, 2H). HRMS (ESI⁺): m/z calcd for C₁₀H₁₃N₈O₃ [M+H]⁺ 293.1105, found 293.1105.

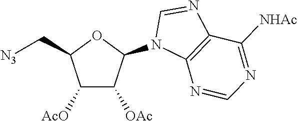 [SMILES: CC(=O)Nc1ncnc2c1ncn2[C@@H]1O[C@H](CN=[N+]=[N-])[C@@H](OC(C)=O)[C@H]1OC(C)=O]

(2R,3R,4R,5R)-2-(6-Acetamido-9H-purin-9-yl)-5-(azidomethyl)tetrahydrofuran-3,4-diyl diacetate (24). To a solution of compound 23 (120 mg, 0.41 mmol, 1.0 eq.) in dry pyridine (6 mL) at 50° C. was added acetic anhydride (0.58 mL, 6.15 mmol, 15.0 eq.). The resultant solution was stirred for 4 h. The mixture was diluted with methanol (10 mL) and the solvents were removed under vacuum. The crude product was purified by column chromatography (70% ethyl acetate in hexane) to give compound 24 as a white solid (95 mg, 56%). ¹H NMR (600 MHz, CDCl₃) δ 9.51 (s, 1H), 8.70 (s, 1H), 8.36 (s, 1H), 6.24 (d, J=5.6 Hz, 1H), 5.95 (t, J=5.7 Hz, 1H), 5.60 (dd, J=5.7, 4.3 Hz, 1H), 4.35 (q, J=4.1 Hz, 1H), 3.73 (dd, J=13.3, 3.6 Hz, 1H), 3.70 (dd, J=13.3, 3.5 Hz, 1H), 2.61 (s, 3H), 2.13 (s, 3H), 2.05 (s, 3H). HRMS (ESI⁺): m/z calcd for C₁₆H₁₉N₈O₆ [M+H]⁺ 419.1422, found 419.1424.

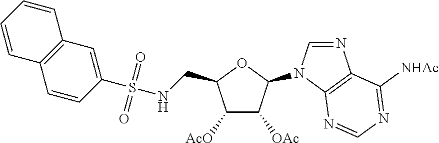 [SMILES: CC(=O)Nc1ncnc2c1ncn2[C@@H]1O[C@H](CNS(=O)(=O)c2ccc3ccccc3c2)[C@@H](OC(C)=O)[C@H]1OC(C)=O]

(2R,3R,4R,5R)-2-(6-Acetamido-9H-purin-9-yl)-5-((naphthalene-2-sulfonamido)methyl)tetrahydrofuran-3,4-diyl diacetate (9). To a solution of compound 24 (84 mg, 0.20 mmol) in dioxane (5 mL) was added 10% Pd/C (17 mg, 20% w/w) and the mixture stirred under an atmosphere of hydrogen for 18 h. The mixture was filtered through Celite, washed with methanol and the solvent removed under vacuum. The crude product 25 was used for next step without further purification. To compound 25 (96 mg, 0.24 mmol, 1.0 eq) in CH₂Cl₂ (15 mL) at room temperature under argon atmosphere was added triethylamine (0.1 mL, 0.73 mmol, 3.0 eq). The mixture was cooled to 0° C. and 2-naphthalenesulfonyl chloride (83 mg, 0.37 mmol, 1.5 eq) was added. The reaction mixture was stirred for 5 h at room temperature. Water (10 mL) was added, and the aqueous layer was extracted with CH₂Cl₂ and the combined organic extracts were washed with brine, dried over Na₂SO₄. The solvent was removed under vacuum and the crude product was purified by column chromatography (95-100% ethyl acetate in hexane) to give compound 9 as a white solid (58 mg, 41%). ¹H NMR (400 MHz, CDCl₃) δ 9.13 (s, 1H), 8.94 (s, 1H), 8.61 (dd, J=9.7, 2.0 Hz, 1H), 8.47 (d, J=2.0 Hz, 1H), 8.08 (s, 1H), 8.01-7.82 (m, 4H), 7.66-7.56 (m, 2H), 5.98 (d, J=6.8 Hz, 1H), 5.95 (t, J=6.2 Hz, 1H), 5.43 (dd, J=5.6, 3.0 Hz, 1H), 4.36 (q, J=2.8 Hz, 1H), 3.50 (ddd, J=13.4, 9.7, 2.5 Hz, 1H), 3.32 (dt, J=13.4, 2.4 Hz, 1H), 2.68 (s, 3H), 2.04 (s, 3H), 2.01 (s, 3H). ¹³C NMR (100 MHz, CDCl₃) δ 171.3, 169.5, 169.2, 152.7, 150.3, 150.2, 142.6, 136.7, 134.9, 132.3, 129.9, 129.4, 128.9, 128.1, 127.7, 123.1, 122.4, 88.5, 82.7, 72.4, 71.8, 60.5, 44.2, 26.0, 20.6, 20.4. HRMS (ESI⁺): m/z calcd for C₂₆H₂₇N₆O₈S [M+H]⁺ 583.1606, found 583.1607.

The synthesis of the pyrrolo[2,1-f][1,2,4-triazine]C-nucleosides is depicted in Scheme 2. The free amine in 7-iodopyrrolo[2,1-f][1,2,4]triazin-4-amine 27 was protected using TMSCI, followed by addition of PhMgCl which removed acidic protons. A magnesium-iodide exchange promoted by ⁱPrMgCl·LiCl followed by addition of ribonolactone 26 gave glycosylated hemiacetal 28 in 42% yield (Scheme 2). The resulting hemiacetal 26 underwent anomeric reduction with triethylsilane and BF₃·OEt₂ to give the stereoselective β-anomer 29. The benzyl groups were removed using boron tribromide to give the corresponding C-nucleoside 30 in 91% yield. After protection of the hydroxyl groups, the resulting nucleoside 31 was converted into the corresponding primary amine 33 following the same synthetic sequence described for compound 12. Amine 33 was coupled with 2-naphthalenesulfonyl chloride to give sulfonamide 34, which was then deprotected to give C-nucleoside sulfonamide 10. Finally, sulfonamide 10 was treated with acetic anhydride to afford its diacetate prodrug 11.

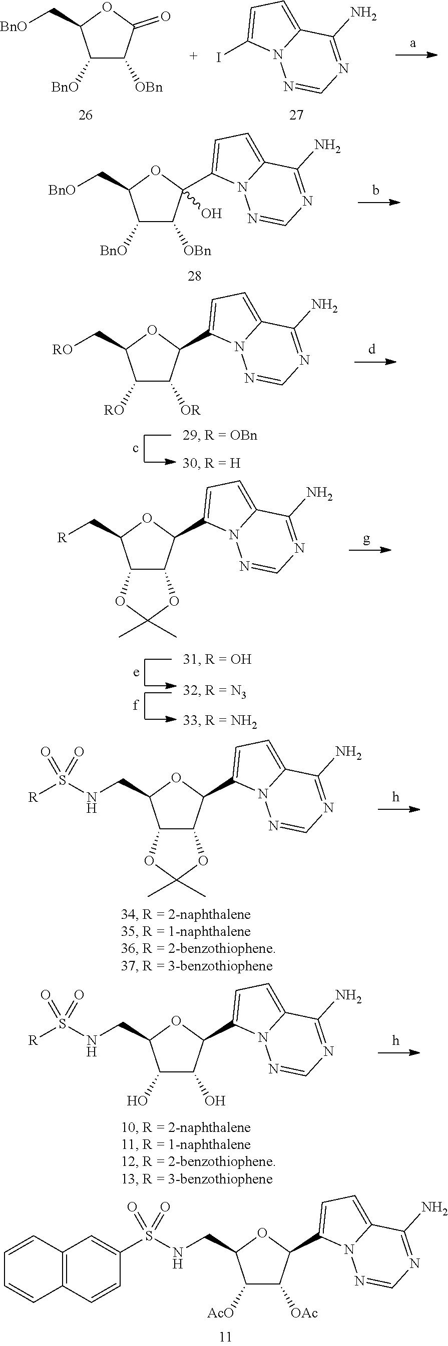

Scheme 2: Reagents and conditions: (a) TMSCl, PhMgCl, ⁱPrMgCl·LiCl, THF, 0° C., 4 h, 42%; (b) BF₃·OEt₂, Et₃SiH, CH₂Cl₂, 0° C., 4 h, 87%; (c) BBr₃, CH₂Cl₂, −78° C., 2 h, 91%; (d) 2,2-dimethoxypropane, H₂SO₄, acetone, rt, 87%; (e) i. DPPA, DBU, dioxane, rt, 16 h; ii. NaN₃, 15-crown-5, 110° C., 18 h, 82% over two steps; (f) H₂, Pd/C, EtOH, 18 h, 95%; (g) sulfonyl chloride, NEt₃, CH₂Cl₂, rt, 18 h; (h) TFA/H₂O (4:1), rt, 3 h; (i) Ac₂O, pyridine, rt, 2 h, 35%.

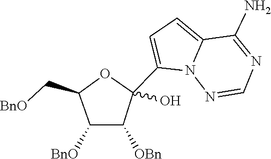 [SMILES: Nc1ncnn2c(C3(O)O[C@H](COCc4ccccc4)[C@@H](OCc4ccccc4)[C@H]3OCc3ccccc3)ccc12]

(3R,4R,5R)-2-(4-Aminopyrrolo[2,1-f][1,2,4]triazin-7-yl)-3,4-bis(benzyloxy)-5-((benzyloxy)methyl)tetrahydrofuran-2-ol (28).¹¹⁻¹² To a solution of 7-iodopyrrolo[2,1-f][1,2,4]triazin-4-amine (27, 1.45 g, 5.58 mmol, 1.0 eq.) in THF (30 mL) under argon atmosphere was added TMSCl (1.41 mL, 11.15 mmol, 2.0 eq.) and the resulting mixture was stirred for 10 min at room temperature. The mixture was cooled to 0° C., and PhMgCl (2 M in THF, 5.57 mL, 11.15 mmol, 2.0 eq.) was added dropwise. The reaction mixture was stirred for 20 min and ⁱPrMgCl·LiCl (1.3 M in THF, 4.28 mL, 5.58 mmol, 1.0 eq.) was added. After 15 min, the reaction mixture was cooled to approximately −20° C. and a solution of 2,3,5-tri-O-benzyl-D-ribono-1,4-lactone (26, 2.33 g, 5.58 mmol, 1.0 eq.) in THE (10 mL) was added dropwise. After 2 h, the reaction mixture was quenched with methanol (5 mL), followed by acetic acid (5 mL) and water (5 mL). The mixture was concentrated under vacuum, then partitioned between EtOAc (100 mL) and 1 M HCl (50 mL). The organic layer was washed with NaHCO₃ (50 mL), brine, dried over Na₂SO₄. The solvent was removed under vacuum and the crude product was purified by column chromatography (70% ethyl acetate in hexane) to give compound 28 as a pale yellow solid (1.3 g, 42%). ¹H NMR (600 MHz, DMSO-d₆) δ 8.01 (s, 2H), 7.98 (s, 1H), 7.38-7.24 (m, 10H), 7.18-7.12 (m, 2H), 7.03-6.97 (m, 2H), 6.94 (d, J=4.8 Hz, 1H), 5.38 (d, J=6.1 Hz, 1H), 4.98 (d, J=5.2 Hz, 1H), 4.68-4.56 (m, 2H), 4.48-4.45 (m, 4H), 4.06-3.98 (m, 2H), 3.93 (dd, J=6.1, 4.4 Hz, 1H), 3.70 (dd, J=10.1, 3.5 Hz, 1H), 3.48 (dd, J=10.1, 6.5 Hz, 1H). HRMS (ESI): m/z calcd for C₃₂H₃₁N₄O₅ [M−H]⁻ 551.2300, found 551.2300.

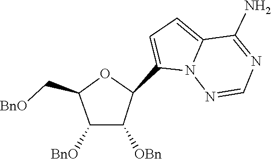 [SMILES: Nc1ncnn2c([C@@H]3O[C@H](COCc4ccccc4)[C@@H](OCc4ccccc4)[C@H]3OCc3ccccc3)ccc12]

7-((2S,3S,4R,5R)-3,4-Bis(benzyloxy)-5-((benzyloxy)methyl)tetrahydrofuran-2-yl)pyrrolo[2,1-f][1,2,4]triazin-4-amine (29).¹¹-2 To a solution of compound 28 (1.1 g, 1.99 mmol, 1.0 eq.) in CH₂Cl₂ (30 mL) at 0° C. under an argon atmosphere was added triethylsilane (1.6 mL, 9.95 mmol, 5.0 eq.), followed by slow addition of BF₃·OEt₂ (0.056 mL, 5.97 mmol, 3 eq.). The resulting solution was stirred for 1 h at 0° C. Saturated aqueous NaHCO₃ solution (30 mL) was added and the aqueous mixture was extracted with CH₂Cl₂. The combined organic extracts were washed with brine, dried over MgSO₄ and the solvent was removed under vacuum. The crude product was purified by column chromatography (70% ethyl acetate in hexane) to give compound 29 as a yellowish oil (0.91 g, 87%). ¹H NMR (600 MHz, CDCl₃) δ 7.92 (s, 1H), 7.34-7.27 (m, 15H), 6.68 (d, J=4.5 Hz, 1H), 6.50 (d, J=4.5 Hz, 1H), 5.72 (s, 2H), 5.70 (d, J=4.2 Hz, 1H), 4.78-4.68 (m, 2H), 4.61 (d, J=12.0 Hz, 1H), 4.58-4.52 (m, 2H), 4.45 (d, J=12.0 Hz, 1H), 4.43-4.39 (m, 1H), 4.29 (t, J=4.6 Hz, 1H), 4.16-4.12 (m, 1H), 3.79 (dd, J=10.7, 3.5 Hz, 1H), 3.67 (dd, J=10.7, 4.1 Hz, 1H). HRMS (ESI⁺): m/z calcd for C₃₂H₃₃N₄O₄ [M+H]⁺ 537.2496, found 537.2495.

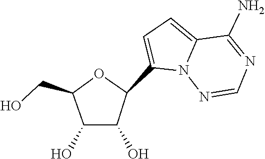 [SMILES: Nc1ncnn2c([C@@H]3O[C@H](CO)[C@@H](O)[C@H]3O)ccc12]

(2S,3R,4S,5R)-2-(4-Aminopyrrolo[2,1-f][1,2,4]triazin-7-yl)-5-(hydroxymethyl)tetrahydrofuran-3,4-diol (30).¹¹⁻¹² To a solution of compound 29 (0.91 g, 1.69 mmol, 1.0 eq.) in CH₂C₀₂ (30 mL) under an atmosphere of argon at −78° C., was added BBr₃ (1.0 M in CH₂Cl₂, 8.5 mL, 8.48 mmol, 5.0 eq.) The mixture was stirred for 2 h at −78° C. Methanol (5 mL) was added and the mixture was warmed to room temperature. The resulting mixture was concentrated under vacuum, and the residue co-evaporated with methanol. The crude product was purified by column chromatography (20% MeOH in CH₂Cl₂) to give compound 30 as a yellowish solid (0.41 g, 91%). ¹H NMR (600 MHz, DMSO-d₆) δ 7.82 (s, 1H), 7.70 (s, 2H), 6.85 (d, J=4.4 Hz, 1H), 6.68 (d, J=4.5 Hz, 1H), 5.10 (d, J=6.5 Hz, 1H), 4.96 (s, 1H), 4.86 (s, 1H), 4.76 (s, 1H), 4.23 (t, J=5.5 Hz, 1H), 3.94 (t, J=4.9 Hz, 1H), 3.78 (q, J=4.5 Hz, 1H), 3.54 (dd, J=11.7, 4.0 Hz, 1H), 3.45 (dd, J=11.7, 4.4 Hz, 1H). HRMS (ESI⁺): m/z calcd for C₁₁H₁₅N₄O₄ [M+H]⁺ 267.1088, found 267.1088.

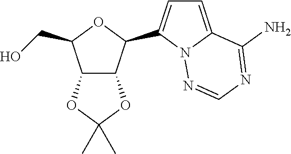 [SMILES: CC1(C)O[C@@H]2[C@H](O1)[C@@H](CO)O[C@H]2c1ccc2c(N)ncnn12]

((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methanol (31). To a solution of compound 30 (0.56 g, 2.10 mmol, 1.0 eq.) in acetone (20 mL) was added 2,2-dimethoxypropane (1.29 mL, 10.5 mmol, 5.0 eq.), followed by addition of sulfuric acid (18 M, 0.15 mL, 2.73 mmol, 1.3 eq.). The reaction mixture was stirred for 30 min, and was warmed to 45° C. After 6 h, a saturated aqueous NaHCO₃ solution (20 mL) was added and the aqueous mixture was extracted with ethyl acetate. The combined organic extracts were washed with brine, dried over MgSO₄ and the solvent was removed under vacuum. The crude product was purified by column chromatography (95-100% ethyl acetate in hexane) to give compound 31 as a colorless oil (0.91 g, 87%). ¹H NMR (400 MHz, CDCl₃) δ 7.90 (s, 1H), 6.69 (d, J=4.5 Hz, 1H), 6.56 (d, J=4.5 Hz, 1H), 5.86 (s, 2H), 5.41-5.13 (m, 2H), 5.03 (dd, J=5.7, 2.4 Hz, 1H), 4.56 (s, 1H), 4.39 (q, J=2.2 Hz, 1H), 3.92 (dd, J=12.2, 2.2 Hz, 1H₁), 3.77 (dd, J=12.3, 2.0 Hz, 1H), 1.63 (s, 3H), 1.36 (s, 3H). ¹³C NMR (100 MHz, CDCl₃) δ 155.8, 147.4, 127.4, 116.0, 114.2, 112.8, 100.3, 84.6, 82.6, 82.3, 81.2, 63.5, 27.9, 25.6. HRMS (ESI⁺): m/z calcd for C₁₄H₁₉N₄O₄ [M+H]⁺ 307.1401, found 307.1402.

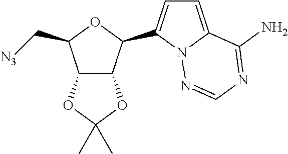 [SMILES: CC1(C)O[C@@H]2[C@H](O1)[C@@H](CN=[N+]=[N-])O[C@H]2c1ccc2c(N)ncnn12]

7-((3aS,4S,6R,6aR)-6-(Azidomethyl)-2,2-dimethyltetrahydrofuro[3,4-d][1,3]dioxol-4-yl)pyrrolo[2,1-f][1,2,4]triazin-4-amine (32). To a solution of compound 31 (0.26 g, 0.85 mmol, 1.0 eq.) in 1,4-dioxane (10 mL) under an atmosphere of argon was added diphenylphosphoryl azide (0.37 mL, 1.69 mmol, 2.0 eq.) and DBU (0.38 mL, 2.54 mmol, 3.0 eq.), and the reaction mixture was stirred for 18 h at room temperature. Sodium azide (0.28 g, 4.24 mmol, 5.0 eq.) and 15-crown-5 (1.6 μL, 0.008 mmol, 0.01 eq.) were added, and the reaction mixture heated at 110° C. for 4 h. The solid was removed by filtration and the solvent was removed under vacuum. The crude product was purified by column chromatography (70% ethyl acetate in hexane) to give compound 32 as a white solid (0.23 g, 82%). ¹H NMR (600 MHz, CDCl₃) δ 7.94 (s, 1H), 6.68 (d, J=4.5 Hz, 1H), 6.62 (d, J=4.5 Hz, 1H), 6.38 (s, 2H), 5.46 (d, J=4.4 Hz, 1H), 5.11 (dd, J=6.7, 4.4 Hz, 1H), 4.79 (dd, J=6.7, 4.5 Hz, 1H), 4.23 (q, J=4.7 Hz, 1H), 3.57 (dd, J=13.0, 4.4 Hz, 1H), 3.44 (dd, J=13.0, 5.2 Hz, 1H), 1.59 (s, 3H), 1.36 (s, 3H). ¹³C NMR (150 MHz, CDCl₃) δ 155.7, 147.6, 127.6, 115.6, 115.0, 110.5, 100.3, 83.9, 83.2, 82.4, 78.6, 52.4, 27.6, 25.7. HRMS (ESI): m/z calcd for C₁₄H₁₆N₇O₃ [M−H]⁻ 330.1320, found 330.1330.

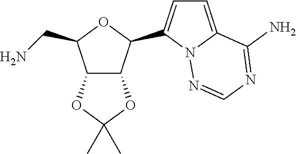 [SMILES: CC1(C)O[C@@H]2[C@H](O1)[C@@H](CN)O[C@H]2c1ccc2c(N)ncnn12]

7-((3aS,4S,6R,6aR)-6-(Aminomethyl)-2,2-dimethyltetrahydrofuro[3,4-d][1,3]dioxol-4-yl)pyrrolo[2,1-f][1,2,4]triazin-4-amine (33). To a solution of compound 32 (0.23 g, 0.69 mmol) in ethanol (20 mL) was added 10% Pd/C (0.046 g, 20% w/w) and the mixture stirred under an atmosphere of hydrogen for 18 h. The mixture was filtered through Celite, washed with methanol and the solvent removed under vacuum. The crude product was purified by column chromatography (20% MeOH in CH₂Cl₂) to give compound 33 as a white solid (0.21 g, 95%). ¹H NMR (600 MHz, CD₃OD) δ 7.81 (s, 1H), 6.85 (d, J=4.5 Hz, 1H), 6.72 (d, J=4.5 Hz, 1H), 5.32 (d, J=4.6 Hz, 1H), 5.14 (dd, J=6.7, 4.7 Hz, 1H), 4.84 (s, 2H), 4.76 (dd, J=6.7, 4.2 Hz, 1H), 4.06 (dt, J=6.4, 4.3 Hz, 1H), 2.87 (dd, J=13.4, 4.4 Hz, 1H), 2.80 (dd, J=13.4, 6.6 Hz, 1H), 1.56 (s, 3H), 1.34 (s, 3H). ¹³C NMR (100 MHz, CD₃OD) δ 157.2, 148.2, 129.2, 116.7, 115.6, 111.7, 102.5, 86.6, 84.9, 84.0, 79.6, 44.7, 27.8, 25.8. HRMS (ESI⁺): m/z calcd for C₁₄H₂₀N₅O₃ [M+H]⁺ 306.1561, found 306.1561.

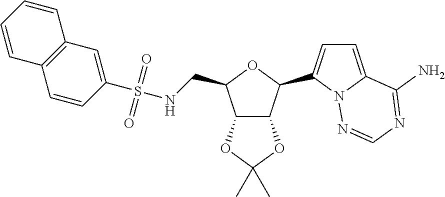 [SMILES: CC1(C)O[C@@H]2[C@H](O1)[C@@H](CNS(=O)(=O)c1ccc3ccccc3c1)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)naphthalene-2-sulfonamide (34). To compound 33 (0.21 g, 0.69 mmol, 1.0 eq.) in CH₂Cl₂ (30 mL) under argon atmosphere at room temperature was added triethylamine (0.29 mL, 2.06 mmol, 3.0 eq.). The mixture was cooled to 0° C. and 2-naphthalenesulfonyl chloride (0.23 g, 1.03 mmol, 1.5 eq.) was added. The reaction mixture was stirred for 24 h at room temperature. Water (20 mL) was added, and the aqueous layer was extracted with CH₂Cl₂ and the combined organic extracts were washed with brine, dried over Na₂SO₄. The solvent was removed under vacuum and the crude product was purified by column chromatography (5% MeOH in CH₂Cl₂) to give compound 34 as a white solid (0.27 g, 61%). ¹H NMR (600 MHz, CDCl₃) δ 8.42 (d, J=1.9 Hz, 1H), 8.32 (s, 1H), 7.93-7.89 (m, 2H), 7.85 (d, J=8.2 Hz, 1H), 7.83 (d, J=9.2 Hz, 1H), 7.78 (dd, J=8.7, 1.9 Hz, 1H), 7.61-7.56 (m, 2H), 6.66 (d, J=4.4 Hz, 1H), 6.59 (d, J=4.4 Hz, 1H), 6.19 (s, 2H), 5.26 (t, J=6.4 Hz, 1H), 5.02 (d, J=6.4 Hz, 1H), 4.84 (dd, J=6.4, 2.5 Hz, 1H), 4.36 (d, J=2.6 Hz, 1H), 3.48-3.41 (m, 1H), 3.17 (dd, J=12.5, 2.9 Hz, 1H), 1.56 (s, 3H), 1.22 (s, 3H). ¹³C NMR (150 MHz, CDCl₃) δ 156.0, 147.9, 136.8, 134.9, 132.3, 129.7, 129.3, 128.8, 128.4, 128.0, 127.6, 126.5, 122.3, 116.2, 114.6, 113.2, 100.4, 82.9, 81.7, 81.6, 81.3, 45.3, 27.8, 25.4. HRMS (ESI⁺): m/z calcd for C₂₄H₂₆N₅O₅S [M+H]⁺ 496.1649, found 496.1647.

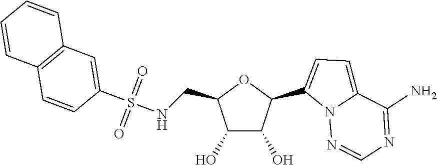 [SMILES: Nc1ncnn2c([C@@H]3O[C@H](CNS(=O)(=O)c4ccc5ccccc5c4)[C@@H](O)[C@H]3O)ccc12]

N-(((2R,3S,4R,5S)-5-(4-Aminopyrrolo[2,1-f][1,2,4]triazin-7-yl)-3,4-dihydroxytetrahydrofuran-2-yl)methyl)naphthalene-2-sulfonamide (10). To compound 34 (100 mg, 0.20 mmol) was added a solution of TFA/H₂O (5 mL, 4:1) at 0° C. The reaction mixture was stirred at 0° C. for 30 min, allowed to warm to room temperature and stirred for a further 4 h. The reaction mixture was diluted with methanol (20 mL) and the solvents were removed under vacuum. The crude product was purified by column chromatography (5% MeOH in CH₂Cl₂) to give compound 10 as a white solid (54 mg, 59%). ¹H NMR (400 MHz, CD₃OD) δ 8.42 (d, J=2.0 Hz, 1H), 7.99 (t, J=8.4 Hz, 2H), 7.94 (d, J=6.8 Hz, 2H), 7.81 (dd, J=8.6, 1.9 Hz, 1H), 7.72-7.57 (m, 2H), 6.84 (d, J=4.5 Hz, 1H), 6.67 (d, J=4.4 Hz, 1H), 5.08 (d, J=7.6 Hz, 1H), 4.70 (dd, J=7.6, 5.7 Hz, 1H), 4.13 (dd, J=5.7, 3.2 Hz, 1H), 4.08 (q, J=3.6 Hz, 1H), 3.25 (dd, J=13.0, 3.8 Hz, 1H), 3.15 (dd, J=13.0, 3.6 Hz, 1H). ¹³C NMR (100 MHz, CD₃OD) δ 157.3, 148.2, 138.3, 136.2, 133.6, 130.6, 130.2, 129.8, 129.1, 129.0, 128.9, 128.7, 123.3, 117.0, 113.3, 102.8, 84.6, 79.3, 74.2, 73.8, 46.4. HRMS (ESI⁺): m/z calcd for C₂₁H₂₂N₅O₅S [M+H]⁺ 456.1336, found 456.1336.

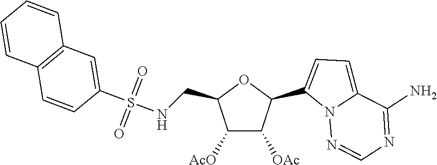

(2S,3S,4R,5R)-2-(4-Aminopyrrolo[2,1-f][1,2,4]triazin-7-yl)-5-((naphthalene-2-sulfonamido)methyl)tetrahydrofuran-3,4-diyl diacetate (11). To a solution of compound 10 (51 mg, 0.11 mmol, 1.0 eq.) in dry pyridine (6 mL) at room temperature was added acetic anhydride (0.12 mL, 0.67 mmol, 6.0 eq.). The resultant solution was stirred for 2 h. The mixture was diluted with methanol (10 mL) and the solvents were removed under vacuum. The crude product was purified by column chromatography (65% ethyl acetate in hexane) to give compound 11 as a white solid (21 mg, 35%). ¹H NMR (400 MHz, CDCl₃) δ 8.45 (d, J=1.4 Hz, 1H), 8.21 (s, 1H), 7.98-7.86 (m, 3H), 7.84 (dd, J=8.6, 1.9 Hz, 1H), 7.68-7.55 (m, 3H), 6.70 (d, J=4.5 Hz, 1H), 6.63 (d, J=4.5 Hz, 1H), 5.93 (dd, J=8.1, 5.9 Hz, 1H), 5.39 (dd, J=5.9, 3.2 Hz, 3H), 5.22 (d, J=8.0 Hz, 1H), 4.25 (q, J=3.0 Hz, 1H), 3.43 (dd, J=12.5, 3.2 Hz, 1H), 3.24 (dd, J=12.5, 3.2 Hz, 1H), 2.06 (s, 3H), 1.98 (s, 3H). ¹³C NMR (100 MHz, CDCl₃) δ 169.9, 169.5, 155.4, 147.5, 136.6, 135.0, 132.3, 129.8, 129.4, 128.8, 128.7, 128.0, 127.6, 125.5, 122.5, 116.2, 113.5, 100.8, 81.7, 76.4, 73.3, 71.4, 44.6, 20.8, 20.6. HRMS (ESI⁺): m/z calcd for C₂₅H₂₆N₅O₇S [M+H]540.1547, found 540.1548.

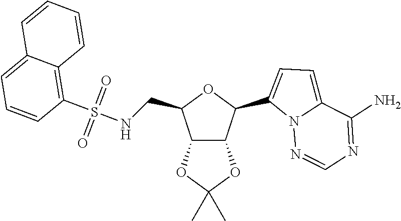 [SMILES: CC1(C)O[C@@H]2[C@H](O1)[C@@H](CNS(=O)(=O)c1cccc3ccccc13)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)naphthalene-1-sulfonamide (35). Compound 35 was prepared in a manner similar to that described for compound 34. ¹H NMR (600 MHz, CDCl₃) δ 8.95-8.66 (m, 1H), 8.36 (s, 1H), 8.28 (dd, J=7.3, 1.3 Hz, 1H), 8.05 (d, J=8.3 Hz, 1H), 8.00-7.85 (m, 2H), 7.64-7.48 (m, 3H), 6.66 (d, J=4.6 Hz, 1H), 6.63 (d, J=4.5 Hz, 1H), 6.12 (s, 2H), 5.20 (t, J=6.5 Hz, 1H), 4.96 (d, J=6.5 Hz, 1H), 4.35 (dd, J=6.4, 2.3 Hz, 1H), 4.26 (t, J=2.4 Hz, 1H), 3.30 (dd, J=12.6, 2.9 Hz, 1H), 3.22 (ddd, J=12.5, 9.5, 2.0 Hz, 1H), 1.50 (s, 4H), 0.98 (s, 3H). HRMS (ESI⁺): m/z calcd for C₂₄H₂₆N₅O₅S [M+H]⁺ 496.1649, found 496.1647.

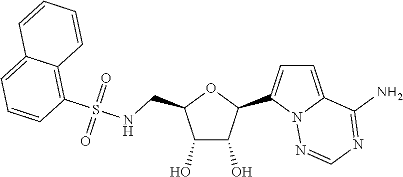 [SMILES: Nc1ncnn2c([C@@H]3O[C@H](CNS(=O)(=O)c4cccc5ccccc45)[C@@H](O)[C@H]3O)ccc12]

N-(((2R,3S,4R,5S)-5-(4-Aminopyrrolo[2,1-f][1,2,4]triazin-7-yl)-3,4-dihydroxytetrahydrofuran-2-yl)methyl)naphthalene-1-sulfonamide (12). Compound 12 was prepared in a manner similar to that described for compound 10. ¹H NMR (400 MHz, CD₃OD) δ 8.81-8.61 (m, 1H), 8.23 (dd, J=7.4, 1.3 Hz, 1H), 8.13 (d, J=8.3 Hz, 1H), 8.04-7.97 (m, 1H), 7.94 (s, 1H), 7.60-7.51 (m, 3H), 6.87 (d, J=4.4 Hz, 1H), 6.65 (d, J=4.5 Hz, 1H), 5.02 (d, J=7.4 Hz, 1H), 4.66 (dd, J=7.5, 5.7 Hz, 1H), 4.00 (d, J=3.5 Hz, 1H), 3.92 (dd, J=5.7, 3.3 Hz, 1H), 3.18 (dd, J=13.1, 3.7 Hz, 1H), 3.12 (dd, J=13.1, 3.9 Hz, 1H). ¹³C NMR (100 MHz, CD₃OD) δ 157.2, 147.9, 136.2, 135.8, 135.3, 130.4, 130.1, 129.5, 129.2, 129.0, 128.0, 125.7, 125.3, 117.0, 113.4, 103.0, 84.6, 79.4, 74.1, 73.7, 48.4. HRMS (ESI⁺): m/z calcd for C₂₁H₂₂N₅O₅S [M+H]⁺ 456.1336, found 456.1336.

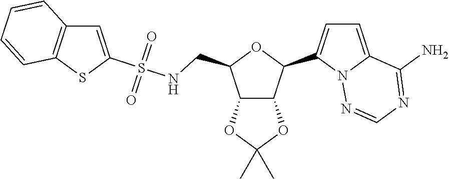 [SMILES: CC1(C)O[C@@H]2[C@H](O1)[C@@H](CNS(=O)(=O)c1cc3ccccc3s1)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)benzo[b]thiophene-2-sulfonamide (36). Compound 36 was prepared in a manner similar to that described for compound 34. ¹H NMR (600 MHz, CDCl₃) δ 8.33 (t, J=1.8 Hz, 1H), 8.06 (d, J=9.5 Hz, 1H), 7.83 (d, J=2.5 Hz, 2H), 7.79 (s, 1H), 7.46-7.40 (m, 2H), 6.69 (t, J=3.6 Hz, 1H), 6.58 (d, J=3.5 Hz, 1H), 5.54 (s, 2H), 5.35-5.20 (m, 1H), 5.14-4.96 (m, 1H), 4.92-4.79 (m, 1H), 4.43 (s, 1H), 4.18-4.03 (m, 1H), 3.54 (t, J=10.9 Hz, 1H), 3.31 (d, J=12.6 Hz, 1H), 1.58 (d, J=2.4 Hz, 3H), 1.22 (d, J=2.5 Hz, 3H). HRMS (ESI⁻): m/z calcd for C₂₂H₂₂N₅O₅S₂ [M−H]⁻ 500.1068, found 500.1066.

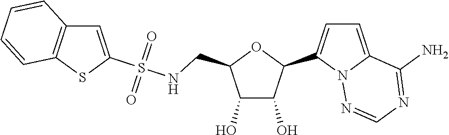 [SMILES: Nc1ncnn2c([C@@H]3O[C@H](CNS(=O)(=O)c4cc5ccccc5s4)[C@@H](O)[C@H]3O)ccc12]

N-(((2R,3S,4R,5S)-5-(4-aminopyrrolo[2,1-f][1,2,4]triazin-7-yl)-3,4-dihydroxytetrahydrofuran-2-yl)methyl)benzo[b]thiophene-2-sulfonamide (13). Compound 13 was prepared in a manner similar to that described for compound 10. ¹H NMR (400 MHz, CD₃OD) δ 7.95 (s, 1H), 7.93-7.83 (m, 3H), 7.53-7.39 (m, 2H), 6.85 (d, J=4.5 Hz, 1H), 6.69 (d, J=4.5 Hz, 1H), 5.08 (d, J=7.8 Hz, 1H), 4.75 (dd, J=7.8, 5.6 Hz, 1H), 4.18 (dd, J=5.6, 2.9 Hz, 1H), 4.15 (q, J=3.2 Hz, 1H), 3.36 (dd, J=12.9, 3.4 Hz, 1H), 3.25 (dd, J=12.9, 3.5 Hz, 1H). ¹³C NMR (100 MHz, CD₃OD) δ 157.4, 148.3, 142.8, 142.4, 139.2, 130.2, 128.7, 128.3, 126.7, 126.6, 123.8, 117.1, 113.5, 102.8, 84.6, 79.4, 74.4, 73.6, 46.7. HRMS (ESI^{T}): m/z calcd for C₁₉H₂₀N₅O₅S₂ [M+H]⁺ 462.0900, found 462.0900.

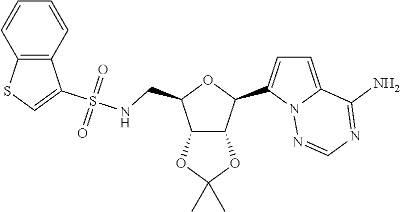 [SMILES: CC1(C)O[C@@H]2[C@H](O1)[C@@H](CNS(=O)(=O)c1csc3ccccc13)O[C@H]2c1ccc2c(N)ncnn12]

N-(((3aR,4R,6S,6aS)-6-(4-Aminopyrrolo[2,1-^{f}][1,2,4]triazin-7-yl)-2,2-dimethyltetrahydrofuro[3,4-d][1,3]dioxol-4-yl)methyl)benzo[b]thiophene-3-sulfonamide (37). Compound 37 was prepared in a manner similar to that described for compound 34. ¹H NMR (600 MHz, CDCl₃) δ 8.36 (s, 1H), 8.25 (s, 1H), 8.23-8.18 (m, 1H), 7.94 (d, J=8.2 Hz, 1H), 7.91-7.84 (m, 1H), 7.40 (dd, J=6.1, 3.1 Hz, 2H), 6.67 (d, J=4.5 Hz, 1H), 6.59 (d, J=4.5 Hz, 1H), 6.00 (s, 2H), 5.18 (t, J=6.5 Hz, 1H), 4.98 (d, J=6.4 Hz, 1H), 4.49 (dd, J=6.5, 2.4 Hz, 1H), 4.31 (d, J=2.5 Hz, 1H), 3.40-3.26 (m, 2H), 1.52 (s, 3H), 1.04 (s, 3H). HRMS (ESI): m/z calcd for C₂₂H₂₂N₅O₅S [M−H]⁻ 500.1068, found 500.1068.

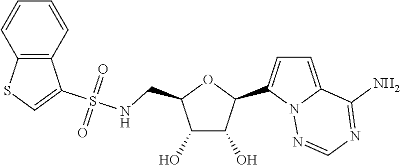 [SMILES: Nc1ncnn2c([C@@H]3O[C@H](CNS(=O)(=O)c4csc5ccccc45)[C@@H](O)[C@H]3O)ccc12]

N-(((2R,3S,4R,5S)-5-(4-Aminopyrrolo[2,1-f][1,2,4]triazin-7-yl)-3,4-dihydroxytetrahydrofuran-2-yl)methyl)benzo[b]thiophene-3-sulfonamide (14). Compound 14 was prepared in a manner similar to that described for compound 10. ¹H NMR (400 MHz, CD₃OD) δ 8.38 (s, 1H), 8.24-8.11 (m, 1H), 7.99-7.96 (m, 1H), 7.93 (s, 1H), 7.52-7.39 (m, 2H), 6.85 (d, J=4.5 Hz, 1H), 6.65 (d, J=4.5 Hz, 1H), 5.02 (d, J=7.4 Hz, 1H), 4.63 (dd, J=7.4, 5.6 Hz, 1H), 4.02 (q, J=3.6 Hz, 1H), 3.97 (dd, J=5.7, 3.5 Hz, 1H), 3.22 (d, J=3.8 Hz, 2H). ¹³C NMR (100 MHz, CD₃OD) δ 157.4, 148.3, 142.0, 135.7, 135.0, 129.1, 126.7, 126.5, 124.1 (×2), 117.0, 113.2, 102.7, 84.4, 79.4, 74.1, 73.7, 46.2. HRMS (ESI⁺): m/z calcd for C₁₉H₂₀N₅O₅S₂ [M+H]⁺ 462.0900, found 462.0901.

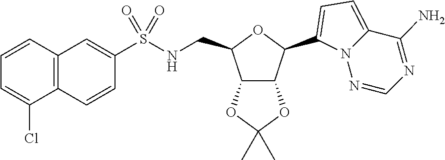 [SMILES: CC1(C)O[C@@H]2[C@H](O1)[C@@H](CNS(=O)(=O)c1ccc3c(Cl)cccc3c1)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-5-chloronaphthalene-2-sulfonamide (38). Compound 38 was prepared in a manner similar to that described for compound 34. White solid, 62 mg, 71%. ¹H NMR (400 MHz, CDCl₃) δ 8.41 (d, J=1.8 Hz, 1H), 8.27 (d, J=8.6 Hz, 2H), 8.02-8.00 (m, 1H), 7.88 (dd, J=8.9, 1.8 Hz, 1H), 7.80-7.77 (m, 1H), 7.62 (dd, J=7.6, 1.1 Hz, 1H), 7.42 (t, J=7.9 Hz, TH), 6.73 (brs, 1H), 6.60 (q, J=2.6 Hz, 2H), 5.24 (t, J=6.2 Hz, 1H), 5.03 (d, J=6.1 Hz, 1H), 4.81 (dd, J=6.5, 2.6 Hz, TH), 4.33 (q, J=2.7 Hz, TH), 3.45 (dd, J=12.8, 6.2 Hz, 1H), 3.18 (dd, J=12.6, 3.1 Hz, 1H), 1.54 (s. 3H), 1.20 (s, 3H). ¹³C NMR (100 MHz, CDCl₃) δ 156.1, 147.7, 137.7, 133.4, 132.1 (×2), 128.8, 128.41, 128.40, 127.5, 126.4 (×2), 123.4, 116.1, 114.6, 112.9, 100.6, 82.7, 81.7, 81.6, 81.0, 45.2, 27.7, 25.3. HRMS (ESI⁺): m/z calcd for C₂₄H₂₅C₁N₅O₅S [M+H]⁺ 530.1259, found 530.1258.

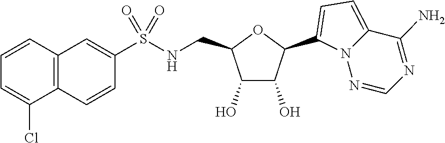 [SMILES: Nc1ncnn2c([C@@H]3O[C@H](CNS(=O)(=O)c4ccc5c(Cl)cccc5c4)[C@@H](O)[C@H]3O)ccc12]

N-(((2R,3S,4R,5S)-5-(4-aminopyrrolo[2,1-f][1,2,4]triazin-7-yl)-3,4-dihydroxytetrahydrofuran-2-yl)methyl)-5-chloronaphthalene-2-sulfonamide (39). Compound 39 was prepared in a manner similar to that described for compound 10. White solid, 35 mg, 94%. ¹H NMR (400 MHz, CD₃OD) δ 8.42 (d, J=1.8 Hz, 1H), 8.25 (d, J=8.9 Hz, 1H), 7.93-7.87 (m, 3H), 7.69 (dd, J=7.5, 1.0 Hz, 1H), 7.49 (t, J=7.9 Hz, 1H), 7.12 (d. J=4.6 Hz, 1H), 6.75 (d, J=4.6 Hz, 1H), 5.12 (d, J=6.2 Hz, 1H), 4.42 (t, J=5.8 Hz, 1H), 4.07 (t, J=5.0 Hz, 1H), 4.00 (dd, J=8.7, 5.2 Hz, 1H), 3.29 (dd, J=11.4, 1.6 Hz, 1H), 3.22 (dd, J=13.7, 5.5 Hz, 1H). ¹³C NMR (100 MHz, CD₃OD) δ 152.1, 139.9, 139.6, 134.7, 134.3, 133.0, 132.8, 129.9, 129.6, 129.4, 128.6 (×2), 126.6, 124.8, 114.0, 108.9, 84.0, 78.5, 75.1, 73.7, 46.3. HRMS (ESI^{T}): m/z calcd for C₂₁H₂₁ClN₅O₅S [M+H]⁺ 490.0946, found 490.0945.

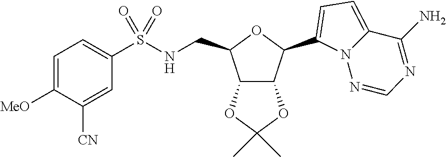 [SMILES: COc1ccc(S(=O)(=O)NC[C@H]2O[C@@H](c3ccc4c(N)ncnn34)[C@@H]3OC(C)(C)O[C@@H]32)cc1C#N]

N-(((3aR,4R,6S,6aS)-6-(4-aminopyrrolo[2,1-f][1,2,4]triazin-7-yl)-2,2-dimethyltetrahydrofuro[3,4-d][1,3]dioxol-4-yl)methyl)-3-cyano-4-methoxybenzenesulfonamide (40). Compound 40 was prepared in a manner similar to that described for compound 34. White solid, 107 mg, 93%. ¹H NMR (400 MHz, CDCl₃) δ 8.12 (s, 1H), 7.99 (d, J=2.4 Hz, 1H), 7.94 (dd, J=8.9, 2.3 Hz, 1H), 7.85-7.83 (m, 1H), 7.00 (d, J=9.0 Hz, 1H), 6.71-6.70 (m, 1H), 6.61 (q, J=4.5 Hz, 2H), 5.22 (t, J=6.2 Hz, 1H), 5.04 (d, J=5.9 Hz, 1H), 4.90 (dd, J=6.5, 2.8 Hz, 1H), 4.33 (q, J=2.7 Hz, 1H), 3.92 (s, 3H), 3.38 (dd, J=13.1, 5.1 Hz, 1H), 3.10 (dd, J=12.3, 3.0 Hz, 1H), 1.57 (s, 3H), 1.30 (s, 3H). BC NMR (100 MHz, CDCl₃) δ 163.8, 156.0, 147.6, 133.4, 133.0, 132.5, 126.3, 116.0, 115.0, 114.7, 112.9, 112.0, 102.5, 100.7, 82.6, 81.7, 81.5, 81.0, 56.8, 45.1, 27.7, 25.4. HRMS (ESI⁺): m/z calcd for C₂₂H₂₅N₆O₆S [M+H]⁺ 501.1551, found 501.1551.

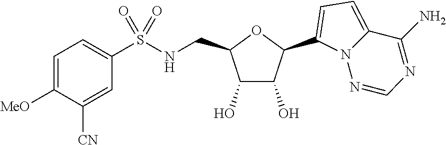 [SMILES: COc1ccc(S(=O)(=O)NC[C@H]2O[C@@H](c3ccc4c(N)ncnn34)[C@H](O)[C@@H]2O)cc1C#N]

N-(((2R,3S,4R,5S)-5-(4-aminopyrrolo[2,1-f][1,2,4]triazin-7-yl)-3,4-dihydroxytetrahydrofuran-2-yl)methyl)-3-cyano-4-methoxybenzenesulfonamide (41). Compound 41 was prepared in a manner similar to that described for compound 10. White solid, 29 mg, 90%. ¹H NMR (400 MHz, CD₃OD) a 8.04-8.01 (m, 2H), 7.93 (s, 1H), 7.24 (d, J=9.6 Hz, 1H), 7.08 (d, J=4.6 Hz, 1H), 6.76 (d, J=4.6 Hz, 1H), 5.18 (d, J=6.7 Hz, 1H), 4.60 (t, J=6.3 Hz, 1H), 4.19 (dd, J=5.9, 4.1 Hz, 1H), 4.06 (q, J=4.2 Hz, 1H), 3.99 (s, 3H), 3.20 (d, J=4.3 Hz, 2H). HRMS (ESI⁺): m/z calcd for C₁₉H₂₁N₆O₆S [M+H]⁺ 461.1238, found 461.1230.

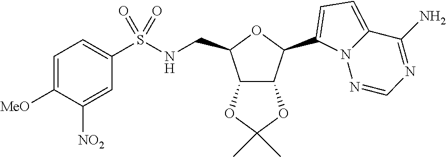 [SMILES: COc1ccc(S(=O)(=O)NC[C@H]2O[C@@H](c3ccc4c(N)ncnn34)[C@@H]3OC(C)(C)O[C@@H]32)cc1[N+](=O)[O-]]

N-(((3aR,4R,6S,6aS)-6-(4-aminopyrrolo[2,1-f][1,2,4]triazin-7-yl)-2,2-dimethyltetrahydrofuro[3,4-d][1,3]dioxol-4-yl)methyl)-4-methoxy-3-nitrobenzenesulfonamide (42). Compound 42 was prepared in a manner similar to that described for compound 34. White solid, 90 mg, 88%. ¹H NMR (400 MHz, CDCl₃) δ 8.26 (d, J=2.3 Hz, 1H), 8.10 (s, 1H), 7.93 (dd, J=8.9, 2.3 Hz, 2H), 7.12 (d, J=9.0 Hz, 1H), 6.79 (brs, 1H), 6.59 (q, J=4.5 Hz, 2H), 5.22 (t, J=6.2 Hz, 1H), 5.04 (d, J=5.9 Hz, 1H), 4.90 (dd, J=6.5, 2.9 Hz, 1H), 4.33 (q, J=2.7 Hz, 1H), 3.94 (s, 3H), 3.40 (d, J=12.3 Hz, 1H), 3.14 (dd, J=12.4, 3.0 Hz, 1H), 1.56 (s, 3H), 1.30 (s, 3H). ¹³C NMR (100 MHz, CDCl₃) δ 156.0, 155.7, 147.6, 138.9, 132.7, 131.9, 126.3, 125.0, 116.0, 114.7, 114.3, 112.8, 100.7, 82.6, 81.7, 81.5, 80.9, 57.1, 45.1, 27.7, 25.4. HRMS (ESI): m/z calcd for C₂₁H₂₅N₆O₈S [M+H]⁺ 521.1449, found 521.1448.

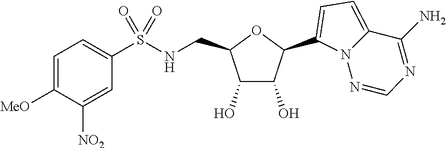 [SMILES: COc1ccc(S(=O)(=O)NC[C@H]2O[C@@H](c3ccc4c(N)ncnn34)[C@H](O)[C@@H]2O)cc1[N+](=O)[O-]]

N-(((2R,3S,4R,5S)-5-(4-aminopyrrolo[2,1-f][1,2,4]triazin-7-yl)-3,4-dihydroxytetrahydrofuran-2-yl)methyl)-4-methoxy-3-nitrobenzenesulfonamide (43). Compound 43 was prepared in a manner similar to that described for compound 10. White solid, 24 mg, 87%. ¹H NMR (400 MHz, CD₃OD) δ 8.21 (d, J=2.4 Hz, 1H), 8.00 (dd, J=8.9, 2.4 Hz, 1H), 7.91 (s, 1H), 7.34 (d, J=9.0 Hz, 1H), 7.10 (d, J=4.6 Hz, 1H), 6.76 (d, J=4.6 Hz, 1H), 5.20 (d, J=6.6 Hz, 1H), 4.62 (t, J=6.3 Hz, 1H), 4.23 (dd, J=5.9, 4.2 Hz, 1H), 4.11-4.05 (m, 1H), 3.98 (s, 3H), 3.22 (dd, J=4.3, 1.9 Hz, 2H). HRMS (ESI⁺): m/z calcd for C₁₈H₂₁N₆O₈S [M+H]⁺ 481.1136, found 481.1142.

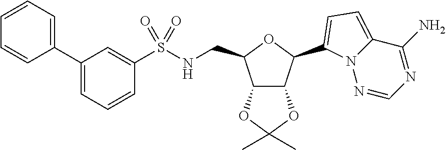 [SMILES: CC1(C)O[C@@H]2[C@H](O1)[C@@H](CNS(=O)(=O)c1cccc(-c3ccccc3)c1)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-[1,1′-biphenyl]-3-sulfonamide (44). Compound 44 was prepared in a manner similar to that described for compound 34. White solid, 57 mg, 72%. ¹H NMR (400 MHz, CDCl₃) δ 8.28 (s, 1H), 8.05 (t, J=1.6 Hz, 1H), 7.83-7.68 (m, 3H), 7.57-7.46 (m, 3H), 7.45-7.30 (m, 3H), 6.68 (d, J=4.4 Hz, 1H), 6.58 (d, J=4.5 Hz, 1H), 6.10 (brs, 2H), 5.27 (t, J=6.4 Hz, 1H), 5.04 (d, J=6.3 Hz, 1H), 4.92 (dd, J=6.4, 2.4 Hz, 1H), 4.38 (d, J=2.2 Hz, 1H), 3.54-3.40 (m, 1H), 3.19 (dd, J=12.3, 2.8 Hz, 1H), 1.59 (s, 3H), 1.30 (s, 3H). ¹³C NMR (100 MHz, CDCl₃) δ 156.0, 148.0, 142.6, 140.6, 139.5, 131.2, 129.9, 129.2 (×2), 128.3, 127.3 (×2), 126.6, 125.7, 125.6, 116.2, 114.8, 113.3, 100.4, 83.0, 81.8, 81.6, 81.4, 45.4, 27.9, 25.6. HRMS (ESI⁺): m/z calcd for C₂₆H₂₈N₅O₅S [M+H]⁺ 522.1806, found 522.1806.

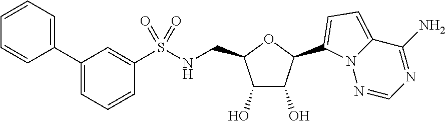 [SMILES: Nc1ncnn2c([C@@H]3O[C@H](CNS(=O)(=O)c4cccc(-c5ccccc5)c4)[C@@H](O)[C@H]3O)ccc12]

N-(((2R,3S,4R,5S)-5-(4-Aminopyrrolo[2,1-f][1,2,4]triazin-7-yl)-3,4-dihydroxytetrahydrofuran-2-yl)methyl)-[1,1′-biphenyl]-3-sulfonamide (45). Compound 45 was prepared in a manner similar to that described for compound 10. White solid, 30 mg, 86%. ¹H NMR (400 MHz, CD₃OD) δ 8.04 (t, J=1.7 Hz, 1H), 7.93 (s, 1H), 7.85-7.77 (m, 2H), 7.59 (t, J=7.8 Hz, 1H), 7.55-7.48 (m, 2H), 7.43-7.30 (m, 3H), 6.84 (d, J=4.4 Hz, 1H), 6.68 (d, J=4.5 Hz, 1H), 5.07 (d, J=7.4 Hz, 1H), 4.69 (dd, J=7.4, 5.7 Hz, 1H), 4.15 (dd, J=5.6, 3.5 Hz, 1H), 4.08 (q, J=3.6 Hz, 1H), 3.26 (dd, J=12.8, 3.8 Hz, 1H), 3.14 (dd, J=12.8, 3.6 Hz, 1H). ¹³C NMR (100 MHz, CD₃OD) δ 157.5, 148.5, 143.8, 142.0, 140.7, 123.2, 131.0, 130.2 (×2), 129.3, 129.1, 128.1 (×2), 126.8, 126.3, 117.2, 113.5, 102.9, 84.5, 79.6, 74.2, 73.8, 46.6. HRMS (ESI⁺): m/z calcd for C₂₃H₂₄N₅O₅S [M+H]⁺ 482.1493, found 482.1499.

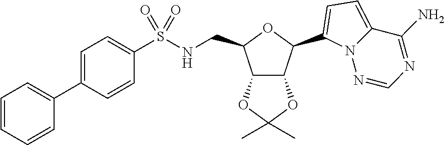 [SMILES: CC1(C)O[C@@H]2[C@H](O1)[C@@H](CNS(=O)(=O)c1ccc(-c3ccccc3)cc1)O[C@H]2c1ccc2c(N)ncnn12]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-[1,1′-biphenyl]-4-sulfonamide (46). Compound 46 was prepared in a manner similar to that described for compound 34. White solid, 59 mg, 75%. ¹H NMR (400 MHz, CDCl₃) δ 8.29 (s, 1H), 7.89 (d, J=8.4 Hz, 2H), 7.77 (d, J=9.5 Hz, 1H), 7.66 (d, J=8.4 Hz, 2H), 7.58-7.52 (m, 2H), 7.49-7.35 (m, 3H), 6.68 (d, J=4.5 Hz, 1H), 6.59 (d, J=4.4 Hz, 1H), 6.14 (brs, 2H), 5.26 (t, J=6.4 Hz, 1H), 5.04 (d, J=6.4 Hz, 1H), 4.90 (dd, J=6.4, 2.3 Hz, 1H), 4.40 (d, J=2.2 Hz, 1H), 3.52-3.40 (m, 1H), 3.19 (dd, J=12.4, 2.8 Hz, 1H), 1.59 (s, 3H), 1.29 (s, 3H). ¹³C NMR (100 MHz, CDCl₃) δ 156.0, 148.0, 145.6, 139.6, 138.6, 129.2 (×2), 128.6, 128.0 (×2), 127.6 (×2), 127.5 (×2), 126.6, 116.2, 114.7, 113.2, 100.4, 83.0, 81.8, 81.6, 81.5, 45.4, 27.9, 25.6. HRMS (ESI⁺): m/z calcd for C₂₆H₂₈N₅O₅S [M+H]⁺ 522.1806, found 522.1804.

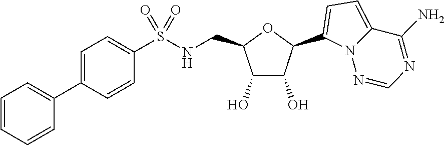 [SMILES: Nc1ncnn2c([C@@H]3O[C@H](CNS(=O)(=O)c4ccc(-c5ccccc5)cc4)[C@@H](O)[C@H]3O)ccc12]

N-(((2R,3S,4R,5S)-5-(4-Aminopyrrolo[2,1-f][1,2,4]triazin-7-yl)-3,4-dihydroxytetrahydrofuran-2-yl)methyl)-[1,1′-biphenyl]-4-sulfonamide (47). Compound 47 was prepared in a manner similar to that described for compound 10. White solid, 31 mg, 84%. ¹H NMR (400 MHz, CD₃OD) δ 7.94 (s, 1H), 7.92-7.87 (m, 2H), 7.78-7.73 (m, 2H), 7.67-7.60 (m, 2H), 7.48-7.42 (m, 2H), 7.42-7.35 (m, 1H), 6.84 (d, J=4.4 Hz, 1H), 6.69 (d, J=4.5 Hz, 1H), 5.07 (d, J=7.6 Hz, 1H), 4.69 (dd, J=7.5, 5.5 Hz, 1H), 4.12 (dd, J=5.5, 3.3 Hz, 1H), 4.08 (q, J=3.5 Hz, 1H), 3.25 (dd, J=12.9, 3.7 Hz, 1H), 3.15 (dd, J=12.9, 3.6 Hz, 1H). ¹³C NMR (100 MHz, CD₃OD) δ 157.5, 148.4, 146.9, 140.7, 140.2, 130.2 (×2), 129.6, 129.1, 128.9 (×2), 128.7 (×2), 128.4 (×2), 117.2, 113.5, 102.9, 84.7, 79.5, 74.3, 73.8, 46.6. HRMS (ESI⁺): m/z calcd for C₂₃H₂₄N₅O₅S [M+H]⁺ 482.1493, found 482.1491.

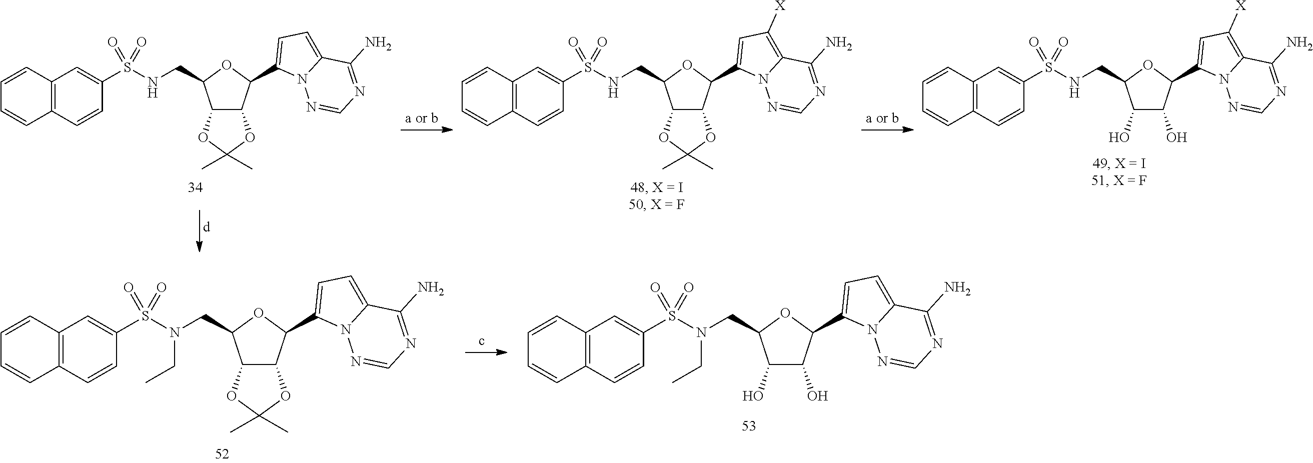

Scheme 3. Reagents and conditions: (a) NIS, TFA, DMF; (b) Selectfluor, NaHCO₃, CH₃CN; (c) TFA/H₂O (4:1), 0° C. to rt; (d) EtOTs, Cs₂CO₃, DMF, 40° C.

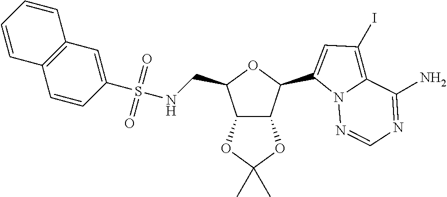 [SMILES: CC1(C)O[C@@H]2[C@H](O1)[C@@H](CNS(=O)(=O)c1ccc3ccccc3c1)O[C@H]2c1cc(I)c2c(N)ncnn12]

N-(((3aR,4R,6S,6aS)-6-(4-Amino-5-iodopyrrolo[2,1-f][1,2,4]triazin-7-yl)-2,2-dimethyltetrahydrofuro[3,4-d][1,3]dioxol-4-yl)methyl)naphthalene-2-sulfonamide (48). To a solution of compound 34 (103 mg, 0.21 mmol, 1.0 eq.) in DMF (5 mL) was added NIS (52 mg, 0.23 mmol, 1.1 eq.) and TFA (3.2 μL, 0.041 mmol), and stirred at room temperature for 18 h. The reaction mixture was diluted with water (10 mL) and extracted with ethyl acetate. The combined organic extracts were washed with water, brine, dried over Na₂SO₄. The solvent was removed under vacuum and the crude product was purified by column chromatography (40% ethyl acetate in hexane) to give compound 48 as a white solid (100 mg, 80%). 1H NMR (600 MHz, CDCl₃) δ 8.41 (d, J=2.0 Hz, 1H), 8.28 (s, 1H), 7.95-7.90 (m, 2H), 7.87 (d, J=7.9 Hz, 1H), 7.77 (dd, J=8.6, 1.9 Hz, 1H), 7.63-7.55 (m, 2H), 7.47 (d, J=9.6 Hz, 1H), 6.82 (s, 1H), 6.62 (s, 2H), 5.21 (t, J=6.4 Hz, 1H), 4.94 (d, J=6.5 Hz, 1H), 4.82 (d, J=6.5 Hz, 1H), 4.34 (d, J=2.6 Hz, 1H), 3.43 (dd, J=12.5, 2.5 Hz, 1H), 3.14 (dd, J=12.5, 3.0 Hz, 1H), 1.55 (s, 3H), 1.23 (s, 3H). HRMS (ESI⁺): m/z calcd for C₂₄H₂₅IN₅O₅S [M+H]⁺ 622.0616, found 622.0616.

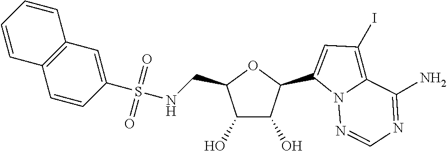 [SMILES: Nc1ncnn2c([C@@H]3O[C@H](CNS(=O)(=O)c4ccc5ccccc5c4)[C@@H](O)[C@H]3O)cc(I)c12]

N-(((2R,3S,4R,5S)-5-(4-Amino-5-iodopyrrolo[2,1-f][1,2,4]triazin-7-yl)-3,4-dihydroxytetrahydrofuran-2-yl)methyl)naphthalene-2-sulfonamide (49). To compound 48 (100 mg, 0.16 mmol) was added a solution of TFA/H₂O (5 mL, 4:1) at 0° C. The reaction mixture was stirred at 0° C. for 30 min, allowed to warm to room temperature and stirred for a further 3 h.

The reaction mixture was diluted with methanol (10 mL) and the solvents were removed under vacuum. The crude product was purified by column chromatography (10% MeOH in CH₂Cl₂) to give compound 49 as a yellow oil (81 mg, 87%). ¹H NMR (400 MHz, CD₃OD) δ 8.41 (d, J=1.9 Hz, 1H), 8.03-7.98 (m, 2H), 7.94 (d, J=8.2 Hz, 1H), 7.88 (s, 1H), 7.81 (dd, J=8.7, 1.9 Hz, 1H), 7.67-7.58 (m, 2H), 6.85 (s, 1H), 5.04 (d, J=6.9 Hz, 1H), 4.52 (dd, J=6.9, 5.5 Hz, 1H), 4.06 (dd, J=5.6, 4.0 Hz, 1H), 4.02 (q, J=4.0 Hz, 1H), 3.23 (dd, J=13.3, 4.5 Hz, 1H), 3.18 (dd, J=13.3, 4.1 Hz, 1H). ¹³C NMR (100 MHz, CD₃OD) δ 157.2, 148.6, 138.5, 136.2, 133.6, 131.7, 130.5, 130.2, 129.8, 129.1, 129.0, 128.6, 123.3, 121.2, 116.3, 84.4, 78.7, 74.2, 73.9, 52.7, 46.3. HRMS (ESI⁺): m/z calcd for C₂₁H₂₁IN₅O₅S [M+H]⁺ 582.0303, found 582.0302.

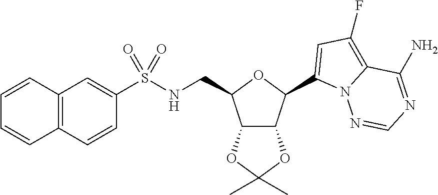 [SMILES: CC1(C)O[C@@H]2[C@H](O1)[C@@H](CNS(=O)(=O)c1ccc3ccccc3c1)O[C@H]2c1cc(F)c2c(N)ncnn12]

N-(((3aR,4R,6S,6aS)-6-(4-Amino-5-fluoropyrrolo[2,1-f][1,2,4]triazin-7-yl)-2,2-dimethyltetrahydrofuro[3,4-d][1,3]dioxol-4-yl)methyl)naphthalene-2-sulfonamide (50). To a solution of compound 34 (100 mg, 0.202 mmol) in anhydrous CH₃CN (2 mL) at rt were added NaHCO₃ (86 mg, 0.24 mmol) and Selectfluor (21 mg, 0.25 mmol). The mixture was allowed to stir for 45 min and quenched with water (0.5 mL) even though compound 34 was not completely consumed. After CH₃CN was removed, the residue was dissolved in EtOAc (15 mL), and the organic layer was washed with water (5 mL) and brine (20 mL) and then dried over Na₂SO₄. After filtration, the filtrate was concentrated, and the residue was purified by flash column chromatography (0% to 10% MeOH/CH₂Cl₂) to give compound 50 as a tan syrup (10 mg, 10%). ¹H NMR (400 MHz, CDCl₃) δ 8.42 (s, 1H), 8.21 (s, 1H), 7.98-7.83 (m, 3H), 7.78 (dd, J=8.6, 1.8 Hz, 1H), 7.67-7.53 (m, 3H), 6.38 (s, 1H), 5.99 (brs, 2H), 5.20 (t, J=6.4 Hz, 1H), 4.88 (d, J=6.4 Hz, 1H), 4.82 (dd, J=6.4, 2.3 Hz, 1H), 4.34 (q, J=2.3 Hz, 1H), 3.50-3.39 (m, 1H), 3.15 (dd, J=12.4, 2.8 Hz, 1H), 1.55 (s, 3H), 1.23 (s, 3H). ¹⁹F NMR (376 MHz, CDCl₃) δ −159.7. HRMS (ESI⁺): m/z calcd for C₂₄H₂₅FN₅O₅S [M+H]⁺ 514.1555, found 514.1563.

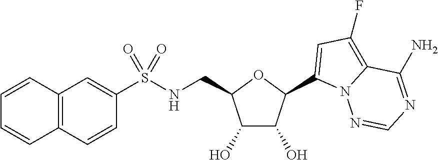 [SMILES: Nc1ncnn2c([C@@H]3O[C@H](CNS(=O)(=O)c4ccc5ccccc5c4)[C@@H](O)[C@H]3O)cc(F)c12]

N-(((2R,3S,4R,5S)-5-(4-Amino-5-fluoropyrrolo[2,1-f][1,2,4]triazin-7-yl)-3,4-dihydroxytetrahydrofuran-2-yl)methyl)naphthalene-2-sulfonamide (51). Compound 50 (10 mg, 0.020 mmol) was deprotected to give compound 51 as a pale solid (6 mg, 65%). ¹H NMR (400 MHz, CD₃OD) δ 8.42 (d, J=1.6 Hz, 1H), 8.04-7.91 (m, 3H), 7.82 (dd, J=8.7, 1.8 Hz, 1H), 7.79 (s, 1H), 7.68-7.57 (m, 2H), 6.44 (s, 1H), 5.02 (d, J=7.0 Hz, 1H), 4.52 (dd, J=7.0, 5.5 Hz, 1H), 4.06 (dd, J=5.5, 3.8 Hz, 1H), 4.02 (q, J=3.9 Hz, 1H), 3.23 (dd, J=13.2, 4.2 Hz, 1H), 3.17 (dd, J=13.2, 3.6 Hz, 1H). ¹⁹F NMR (376 MHz, CD₃OD) δ −161.1. ¹³C NMR (100 MHz, CD₃OD) δ 156.1 (J_{C-F}=3.7 Hz), 148.9, 145.2 (J_{C-F}=246 Hz), 138.6, 136.3, 133.7, 130.7, 130.3, 130.0, 129.3, 129.1, 128.8, 126.2 (J_{C-F}=3.0 Hz), 123.5, 103.0 (J_{C-F}=22.4 Hz), 99.1 (J_{C-F}=14.4 Hz), 84.7, 78.9, 74.4, 74.0, 46.4. HRMS (ESI⁺): m/z calcd for C₂₁H₂₁FN₅O₅S [M+H]⁺ 474.1242, found 474.1245.

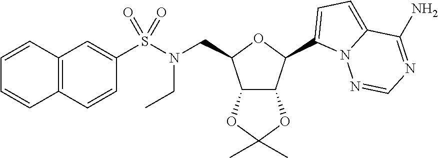 [SMILES: CCN(C[C@H]1O[C@@H](c2ccc3c(N)ncnn23)[C@@H]2OC(C)(C)O[C@@H]21)S(=O)(=O)c1ccc2ccccc2c1]

N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-N-ethylnaphthalene-2-sulfonamide (52). To a solution of compound 34 (100 mg, 0.202 mmol) were added Cs₂CO₃ (99 mg, 0.30 mmol) and EtOTs (61 mg, 0.30 mmol), and the resulting mixture was heated at 40° C. for 15 h. Since TLC showed that the reaction was not complete, additional Cs₂CO₃ (20 mg, 0.061 mmol) and EtOTs (13 mg, 0.065 mmol) were added, and the mixture was heated at 40° C. for 3 h. After the reaction mixture was concentrated, the residue was dissolved in EtOAc (20 mL). The organic layer was washed with water (10×2 mL) and brine (20 mL) and then dried over Na₂SO₄. After filtration, the filtrate was concentrated, and the residue was purified by flash column chromatography (40% to 100% EtOAc/hexanes) to give compound 52 as a white solid (85 mg, 800%). ¹H NMR (400 MHz, CDCl₃) δ 8.36 (d, J=1.0 Hz, 1H), 7.96 (s, 1H), 7.93-7.82 (m, 3H), 7.75 (dd, J=8.7, 1.8 Hz, 1H), 7.65-7.53 (m, 2H), 6.70 (d, J=4.5 Hz, 1H), 6.60 (d, J=4.5 Hz, 1H), 5.71 (brs, 2H), 5.42 (d, J=4.0 Hz, 1H), 5.13 (dd, J=6.6, 4.0 Hz, 1H), 4.96 (dd, J=6.6, 4.3 Hz, 1H), 4.27-4.18 (m, 1H), 3.69 (dd, J=14.4, 6.5 Hz, 1H), 3.45-3.34 (m, 1H), 3.34-3.20 (m, 2H), 1.56 (s, 3H), 1.36 (s, 3H), 1.06 (t, J=7.1 Hz, 3H). ¹³C NMR (100 MHz, CDCl₃) δ 155.5, 147.5, 137.1, 134.9, 132.4, 129.5, 129.4, 128.8, 128.6, 128.4, 128.0, 127.6, 122.8, 115.4, 114.9, 111.0, 100.0, 83.8, 83.5, 83.2, 78.7, 49.1, 43.9, 27.6, 25.8, 13.8. HRMS (ESI⁺): m/z calcd for C₂₆H₃₀N₅O₅S [M+H]⁺ 524.1962, found 524.1955.

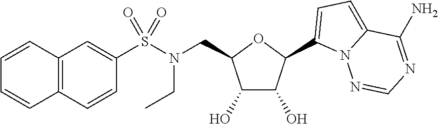 [SMILES: CCN(C[C@H]1O[C@@H](c2ccc3c(N)ncnn23)[C@H](O)[C@@H]1O)S(=O)(=O)c1ccc2ccccc2c1]

N-(((2R,3S,4R,5S)-5-(4-Aminopyrrolo[2,1-f][1,2,4]triazin-7-yl)-3,4-dihydroxytetrahydrofuran-2-yl)methyl)-N-ethylnaphthalene-2-sulfonamide (53). Compound 52 (60 mg, 0.11 mmol) was deprotected to give compound 53 as a white solid (45 mg, 82%). ¹H NMR (400 MHz, DMSO-d6) δ 8.48 (s, 1H), 8.13 (d, J=8.0 Hz, 1H), 8.08 (d, J=8.8 Hz, 1H), 8.03 (d, J=8.0 Hz, 1H), 7.87-7.77 (m, 2H), 7.74-7.59 (m, 4H), 6.84 (d, J=4.4 Hz, 1H), 6.66 (d, J=4.4 Hz, 1H), 5.14 (d, J=5.6 Hz, 1H), 5.10 (d, J=6.0 Hz, 1H), 5.06 (d, J=5.2 Hz, 1H), 4.32-4.22 (m, 1H), 4.02-3.88 (m, 2H), 3.61 (dd, J=14.4, 2.9 Hz, 1H), 3.32-3.13 (m, 3H), 0.97 (t, J=7.0 Hz, 3H). ¹³C NMR (100 MHz, DMSO-d6) δ 155.6, 147.7, 136.8, 134.2, 131.8, 129.3, 129.2, 128.7, 128.5, 127.8, 127.7, 127.5, 122.4, 114.9, 109.5, 100.8, 81.8, 76.3, 73.3, 72.5, 50.0, 43.2, 13.5. HRMS (ESI^{T}): m/z calcd for C₂₃H₂₆N₅O₅S [M+H]⁺ 484.1649, found 484.1633.

### Example 2

The synthesis began with commercially available Vince lactam (A5, (1R,4S)-2-azabicyclo[2.2.1]hept-5-en-3-one) which was converted into the corresponding phenylsulfonamide compound A6 (Scheme 4). Compound A6 was reductively opened to hydroxy sulfonamide A7, which was further acylated with pivaloyl chloride to give cyclopentenylamine intermediate A8. To attach adenine (A9) to the cyclopentene ring, π-allylpalladium complexes chemistry was employed. Combination of [PdCl(C₃H₅)]₂ and P(OⁱPr)₃ generated catalyst complex Pd[P(OiPr)₃]₄, which provided π-allylpalladium intermediate A10.¹ Adenosine moiety was then installed successfully to give the compound A11 in 64% yield. Dihydroxylation, acetonide protection and subsequent ester hydrolysis gave an inseparable approximately 1:2 mixture of diastereomeric compounds A14α and A14β. This diastereomeric mixture was converted into the corresponding mixture of azides A15α/A15β, which was directly used in next reaction without further purification. Reduction of azides A15α/A15β under hydrogenation using 20 wt % Pd/C in ethanol gave separable two fractions (Scheme 5). The first fraction contained an inseparable mixture of primary amine A16α and secondary ethyl amine A17α, and the second fraction contained an inseparable mixture of primary amine A16β and secondary ethyl amine A17β. Generation of secondary amines 17α and 17β was likely due to a palladium-catalyzed amine alkylation with primary alcohol,² in which a primary alcohol was converted to the corresponding aldehyde by Pd/C-catalyzed dehydration. Subsequent imine formation and hydrogenation gave rise to a secondary amine. The mixture of primary amine A16α and ethylamine A17α was treated with 2-naphthalenesulfonyl chloride to give separable sulfonamides A18 and A19, respectively. Similarly, treatment of the mixture of A16β and A17β led to sulfonamides A20 and A21. Finally, removal of the acetonide group using 80% aqueous TFA gave α-carbocyclic nucleoside sulfonamide A1, α-carbocyclic nucleoside ethyl sulfonamide A2, β-carbocyclic nucleoside sulfonamide A3 and β-carbocyclic nucleoside ethyl sulfonamide A4. The relative configurations were confirmed by NOESY experiments, which showed that compound A1 was indeed an α-carbocyclic nucleoside (data not shown) and A3 was a β-carbocyclic nucleoside (data not shown).

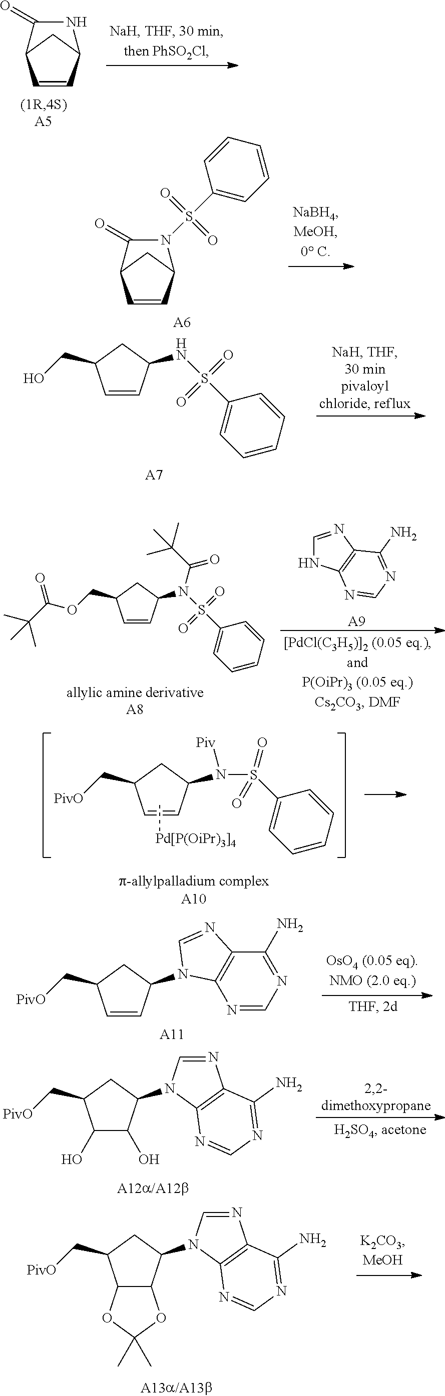

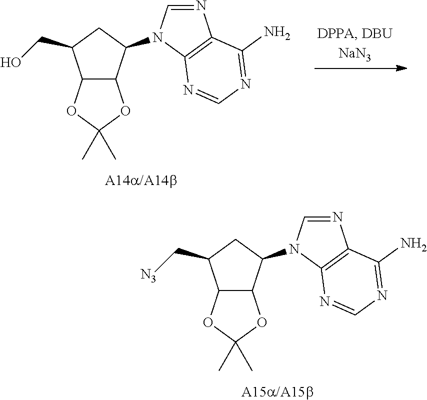 [SMILES: CC1(C)OC2C(O1)[C@H](n1cnc3c(N)ncnc31)C[C@@H]2CO ; CC1(C)OC2C(O1)[C@H](n1cnc3c(N)ncnc31)C[C@@H]2CN=[N+]=[N-]]

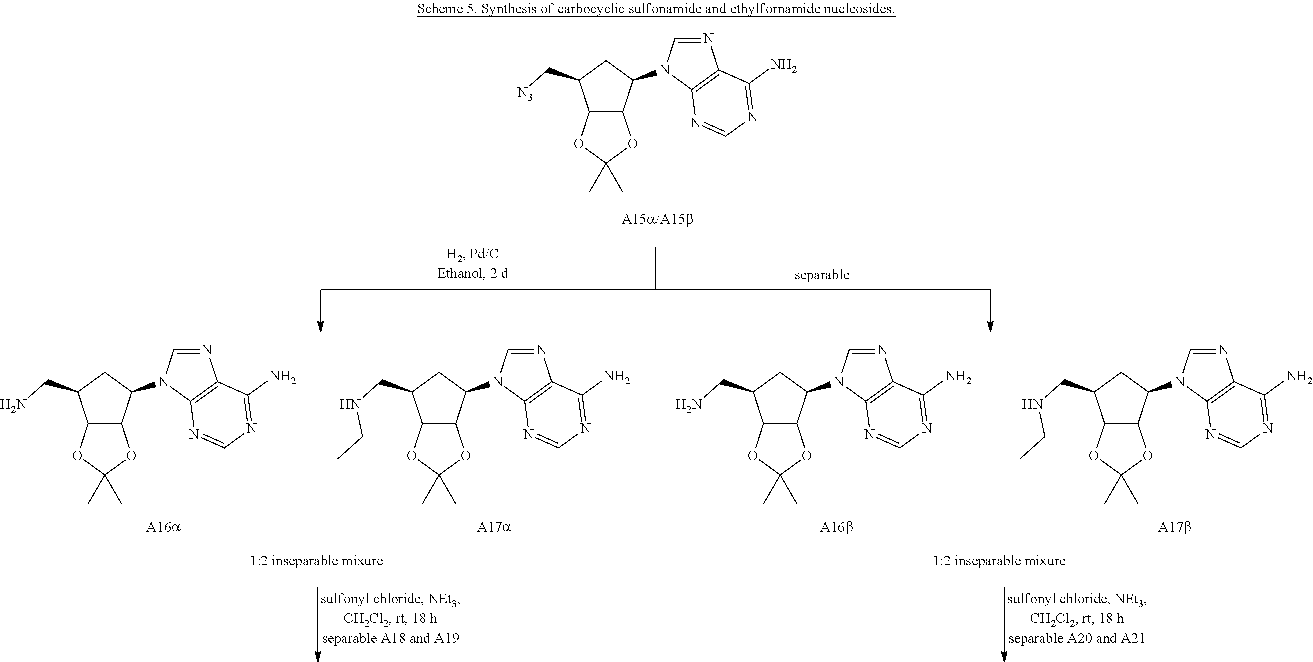 [SMILES: CC1(C)OC2C(O1)[C@H](n1cnc3c(N)ncnc31)C[C@@H]2CN=[N+]=[N-] ; CC1(C)OC2C(O1)[C@H](n1cnc3c(N)ncnc31)C[C@@H]2CN ; CCNC[C@H]1C[C@@H](n2cnc3c(N)ncnc32)C2OC(C)(C)OC21 ; CC1(C)OC2C(O1)[C@H](n1cnc3c(N)ncnc31)C[C@@H]2CN ; CCNC[C@H]1C[C@@H](n2cnc3c(N)ncnc32)C2OC(C)(C)OC21]

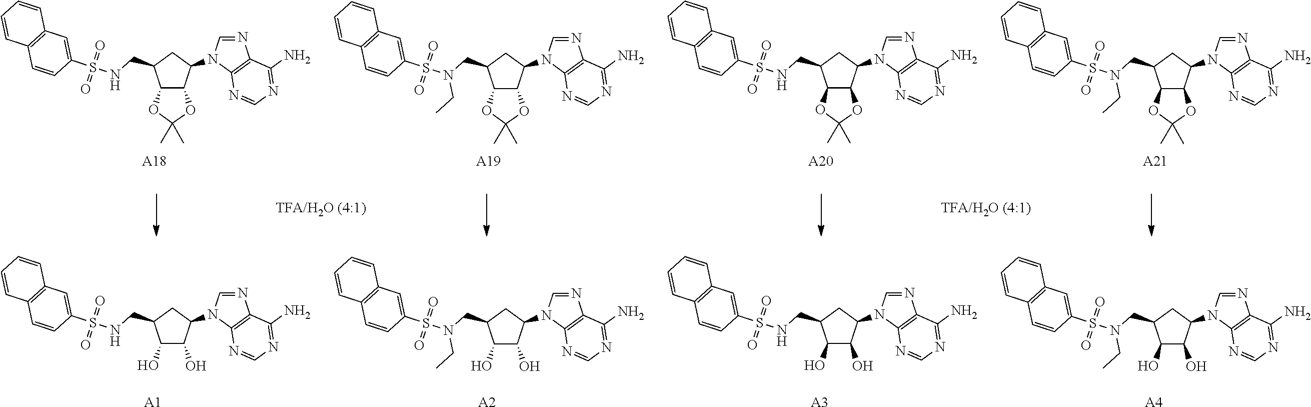

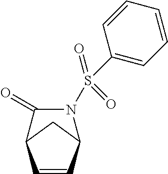 [SMILES: O=C1[C@@H]2C=C[C@@H](C2)N1S(=O)(=O)c1ccccc1]

(1R,4S)-2-(Phenylsulfonyl)-2-azabicyclo[2.2.1]hept-5-en-3-one (A6). To a solution of (1R,4S)-2-azabicyclo[2.2.1]hept-5-en-3-one (A5) (1.52 g, 13.9 mmol, 1.0 eq.) in THF (80 mL) at 0° C. was added NaH (60% dispersion in mineral oil, 0.67 g, 16.7 mmol, 1.2 eq.). The reaction mixture was stirred for 30 min at room temperature and benzenesulfonyl chloride (2.14 mL, 16.7 mmol, 1.2 eq.) was added to the mixture. The reaction mixture was stirred for 3 h at room temperature. Water (50 mL) was added, and the aqueous layer was extracted with CH₂Cl₂ and the combined organic layer was washed with aqueous NaHCO₃ (50 mL), brine, dried over Na₂SO₄. After filtration, the solvent was removed under vacuum and the crude product was purified by column chromatography (40% EtOAc in hexanes) to give compound A6 as a white solid (1.8 g, 53%). 1H NMR (600 MHz, CDCl₃) δ 8.02-7.70 (m, 2H), 7.57 (t, J=7.6 Hz, 1H), 7.46 (t, J=7.8 Hz, 2H), 6.58 (dd, J=5.4, 2.2 Hz, 1H), 6.31 (t, J=3.9 Hz, 1H), 5.00 (d, J=1.9 Hz, 1H₁), 3.33 (d, J=1.5 Hz, 1H), 2.37 (d, J=8.7 Hz, 1H), 2.14 (dt, J=8.8, 1.8 Hz, 1H). HRMS (ESI⁺): m/z calcd for C₁₂H₁₀NO₃S [M−H]⁻ 250.0532, found 250.0531.

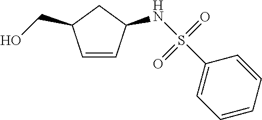 [SMILES: O=S(=O)(N[C@H]1C=C[C@@H](CO)C1)c1ccccc1]

N-((1R,4S)-4-(Hydroxymethyl)cyclopent-2-en-1-yl)benzenesulfonamide (A7). To a solution of compound A6 (1.8 g, 7.2 mmol, 1.0 equiv) in MeOH (40 mL) at 0° C. was added NaBH₄ (1.1 g, 28.9 mmol, 4.0 eq.). The mixture was stirred for 1.5 h at 0° C. and then quenched by dropwise addition of saturated aqueous NH₄Cl (40 mL). The mixture was extracted with EtOAc and the combined organic layer was washed with brine and dried over Na₂SO₄. After filtration, the solvent was removed under vacuum and the crude product was purified by column chromatography (5% MeOH in CH₂Cl₂) to give compound A7 as a colorless oil (1.52 g, 84%). ¹H NMR (600 MHz, CDCl₃) δ 8.29-7.78 (m, 2H), 7.57 (t, J=7.7 Hz, 1H), 7.51 (t, J=7.6 Hz, 2H), 5.72 (dd, J=5.4, 1.8 Hz, 1H), 5.61 (dd, J=5.3, 2.7 Hz, 1H), 5.34 (s, 1H), 4.36 (d, J=8.3 Hz, 1H), 3.65-3.61 (m, 1H), 3.53-3.48 (m, 2H), 2.74 (dd, J=5.9, 3.0 Hz, 1H), 2.35-2.14 (m, 1H), 1.32 (dt, J=14.0, 2.7 Hz, 1H). HRMS (ESI⁺): m/z calcd for C₁₂H₁₄NO₃S [M−H]⁻ 252.0700, found 252.0701.

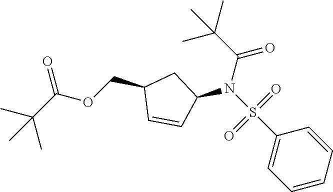 [SMILES: CC(C)(C)C(=O)OC[C@@H]1C=C[C@H](N(C(=O)C(C)(C)C)S(=O)(=O)c2ccccc2)C1]

((1S,4R)-4-(N-(Phenylsulfonyl)pivalamido)cyclopent-2-en-1-yl)methyl pivalate (A8). To a solution of NaH (2.4 g, 60.0 mmol, 10.0 eq.) in THE (30 mL) under argon atmosphere at 0° C. was added a solution of alcohol A7 (1.52 g, 6.0 mmol, 1.0 eq.) in THF (30 mL) over 5 min. The mixture was stirred at room temperature for 30 min and pivolyl chloride (7.3 mL, 60.0 mmol, 10.0 eq.) was added and the mixture was refluxed for 4 h. After a saturated aqueous NH₄Cl solution (30 mL) was added to the quench the reaction, the aqueous mixture was extracted with EtOAc. The combined organic layer was washed with brine and dried over MgSO₄. After filtration, the solvent was removed under vacuum and the crude product was purified by column chromatography (10% EtOAc in hexanes) to give compound A8 as a white solid (1.8 g, 72%). ¹H NMR (600 MHz, CDCl₃) δ 7.93-7.84 (m, 2H), 7.60 (t, J=7.5 Hz, 1H), 7.55-7.47 (m, 2H), 5.79 (d, J=5.6 Hz, 1H), 5.63 (d, J=5.6 Hz, 1H), 4.82 (td, J=7.8, 2.2 Hz, 1H), 3.99 (ddd, J=7.3, 3.4, 1.3 Hz, 2H), 3.02-2.72 (m, 1H), 2.35-2.15 (m, 1H), 1.79 (ddd, J=13.8, 7.7, 6.6 Hz, 1H), 1.33 (s, 9H), 1.17 (d, J=1.4 Hz, 9H). HRMS (ESI): m/z calcd for C₂₂H₃₀NO₅S [M−H]⁻ 420.1850, found 420.1831.

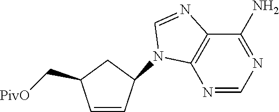

((1S,4R)-4-(6-Amino-9H-purin-9-yl)cyclopent-2-en-1-yl)methyl pivalate (A11). A solution of adenine A9 (0.18 g, 1.37 mmol, 1.2 eq.) and Cs₂CO₃ (0.45 g, 1.37 mmol, 1.2 eq.) in DMF (10 mL) under argon atmosphere at room temperature was stirred for 30 min. To another solution of allylic amine derivative A8 (0.48 g, 1.13 mmol, 1.0 eq.) in DMF (10 mL) were added [PdCl(C₃-H₅)]2 (0.02 g, 0.057 mmol, 0.05 eq.) and P(OiPr)₃ (0.14 mL, 0.57 mmol, 0.5 eq.). After being stirred at room temperature for 30 min, the allylic amine mixture was transferred to the adenine mixture over 5 min at 0° C. and the reaction mixture was stirred for 18 h at room temperature and then quenched with saturated aqueous NH₄Cl. The aqueous mixture was extracted with EtOAc and the combined organic layer was washed with brine and dried over MgSO₄. After filtration, the solvent was removed under vacuum and the crude product was purified by column chromatography (5% MeOH in CH₂Cl₂) to give compound A11 as a white solid (0.23 g, 64%). ¹H NMR (600 MHz, CDCl₃) δ 8.32 (s, 1H), 7.80 (s, 1H), 6.38 (s, 2H), 6.13 (dt, J=5.8, 2.1 Hz, 1H), 6.04-5.91 (m, 1H), 5.72 (tt, J=6.7, 2.2 Hz, 1H), 4.09 (qd, J=10.9, 6.4 Hz, 2H), 3.21-3.08 (m, 1H), 2.87 (dt, J=13.8, 8.4 Hz, 1H), 1.63 (dt, J=13.8, 6.9 Hz, 1H), 1.16 (s, 9H). HRMS (ESI⁺): m/z calcd for C₁₆H₂₂N₅O₂ [M+H]⁺ 316.1768, found 316.1768.

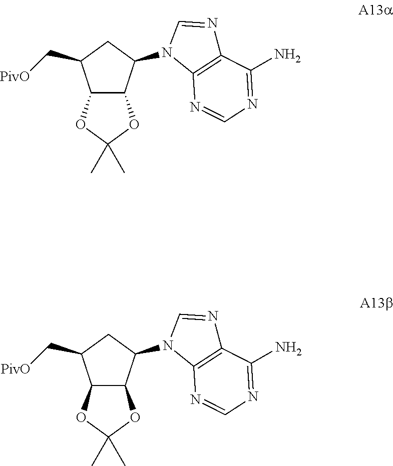

((3aR,4R,6R,6aS)-6-(6-Amino-9H-purin-9-yl)-2,2-dimethyltetrahydro-4H-cyclopenta[d][1,3]dioxol-4-yl)methyl pivalate (A13a) and ((3aS,4R,6R,6aR)-6-(6-Amino-9H-purin-9-yl)-2,2-dimethyltetrahydro-4H-cyclopenta[d][1,3]dioxol-4-yl)methyl pivalate (A13β). To a solution of compound A11 (210 mg, 0.67 mmol, 1.0 equiv) and NMO (156 mg, 1.33 mmol, 2.0 equiv) in 1:1 THF:water mixture (10 mL) at 0° C. was added OsO₄ (0.20 mL, 0.019 mmol, as a 2.5 g/100 mL solution in t-butanol, 0.03 eq.) dropwise. After the resulting mixture was stirred at 0° C. for 24 h, saturated aqueous sodium sulfite solution (10 mL) was added and the mixture stirred for 1 h. The aqueous mixture was extracted with EtOAc and the combined organic layer was washed with brine and dried over MgSO₄. After filtration, the solvent was removed under vacuum and the crude product was directly used in the next reaction.

To a solution of crude diol A12α/A12β (190 mg, 0.54 mmol, 1.0 eq.) in acetone (20 mL) was added 2,2-dimethoxypropane (0.33 mL, 2.72 mmol, 5.0 eq.) and then sulfuric acid (18 M, 0.04 mL, 2.73 mmol, 0.71 eq.). The reaction mixture was stirred for 30 min and at 45° C. for 6 h. A saturated aqueous NaHCO₃ solution (10 mL) was added to quench the reaction and the aqueous mixture was extracted with EtOAc. The combined organic layer was washed with brine and dried over MgSO₄. After filtration, the solvent was removed under vacuum. The crude product was purified by column chromatography (20% MeOH in CH₂Cl₂) to give A13β as a pure fraction and compounds A13α/A13β (0.044 g, 63%) as an inseparable 1:1.1 mixture of diastereomers. Diastereomer A13α: ¹H NMR (600 MHz, CD₃OD) δ 8.23 (s, 1H), 8.22 (s, 1H), 4.83 (d, J=5.2 Hz, 1H), 4.78 (t, J=3.1 Hz, 21H), 4.26-4.19 (m, 2H), 2.39-2.34 (m, 1H), 2.33-2.24 (m, 1H), 2.22-2.15 (m, 1H), 1.48 (s, 3H), 1.27 (s, 3H), 1.22 (s, 9H). Diastereomer A13β: ¹H NMR (400 MHz, CD₃OD) δ 8.19 (s, 1H), 8.18 (s, 1H), 5.12 (dd, J=7.3, 5.8 Hz, 1H), 4.89 (td, J=9.4, 5.8 Hz, 1H), 4.67 (dd, J=7.3, 5.4 Hz, 1H), 4.23 (dd, J=6.2, 1.4 Hz, 2H), 2.60-2.48 (m, 1H), 2.46-2.36 (m, 2H), 1.54 (s, 3H), 1.29 (d, J=0.8 Hz, 3H), 1.23 (s, 9H). ¹³C NMR (100 MHz, MeOD) δ 179.9, 157.4, 153.6, 150.7, 141.9, 120.7, 115.1, 84.7, 82.8, 65.9, 62.8, 44.6, 39.9, 34.8, 27.8, 27.6, 25.4. HRMS (ESI⁺): m/z calcd for C₁₉H₂₈N₅O₄ [M+H]⁺ 390.2136, found 390.2137.

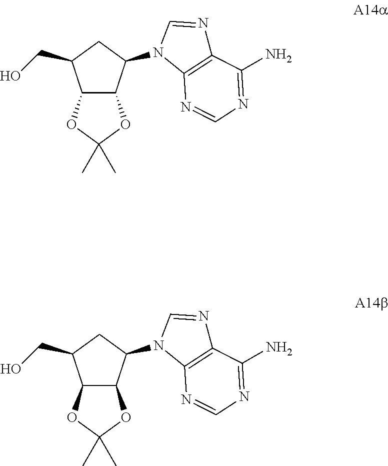 [SMILES: CC1(C)O[C@@H]2[C@@H](CO)C[C@@H](n3cnc4c(N)ncnc43)[C@@H]2O1 ; CC1(C)O[C@H]2[C@@H](CO)C[C@@H](n3cnc4c(N)ncnc43)[C@H]2O1]

((3aR,4R,6R,6aS)-6-(6-Amino-9H-purin-9-yl)-2,2-dimethyltetrahydro-4H-cyclopenta[d][1,3]dioxol-4-yl)methanol (A14α) and ((3aS,4R,6R,6aR)-6-(6-Amino-9H-purin-9-yl)-2,2-dimethyltetrahydro-4H-cyclopenta[d][1,3]dioxol-4-yl)methanol (A14β). To a solution of compounds A13α/A13β (130 mg, 0.33 mmol, 1.0 eq.) in MeOH (10 mL) at 0° C. was added K₂CO₃ (140 mg, 1.00 mmol, 3.0 eq.). The resulting mixture was stirred at 0° C. for 30 min and then heated at 60° C. for 4 h. The solution was concentrated under vacuum and the crude product was purified by column chromatography (15% MeOH in CH₂Cl₂) to give compounds A14α/A14β as a 1:2 inseparable mixture of diastereomers (white solid, 78 mg, 71%). Diastereomer A14α: ¹H NMR (600 MHz, CD₃OD) δ 8.24 (s, 1H), 8.20 (s, 1H), 5.07 (t, J=6.6 Hz, 1H), 4.89 (dt, J=12.4, 6.6 Hz, 1H), 4.66 (dd, J=7.2, 4.8 Hz, 1H), 3.71 (dd, J=10.5, 5.8 Hz, 1H), 3.68 (dd, J=10.5, 5.8 Hz, 1H), 2.44 (dd, J=12.3, 6.6 Hz, 1H), 2.38 (dt, J=11.6, 5.7 Hz, 1H), 2.31 (t, J=11.6 Hz, 1H), 1.55 (s, 3H), 1.31 (s, 3H). Diastereomer A14β: ¹H NMR (600 MHz, CD₃OD) δ 8.22 (s, 1H), 8.21 (s, 1H), 4.83-4.75 (m, 3H), 3.88 (dd, J=10.8, 6.2 Hz, 1H), 3.72 (dd, J=10.7, 5.6 Hz, 1H), 2.21-2.14 (m, 3H), 1.48 (s, 3H), 1.27 (s, 3H). HRMS (ESI⁺): m/z calcd for C₁₄H₂₀N₅O₃ [M+H]⁺ 306.1561, found 306.1561.

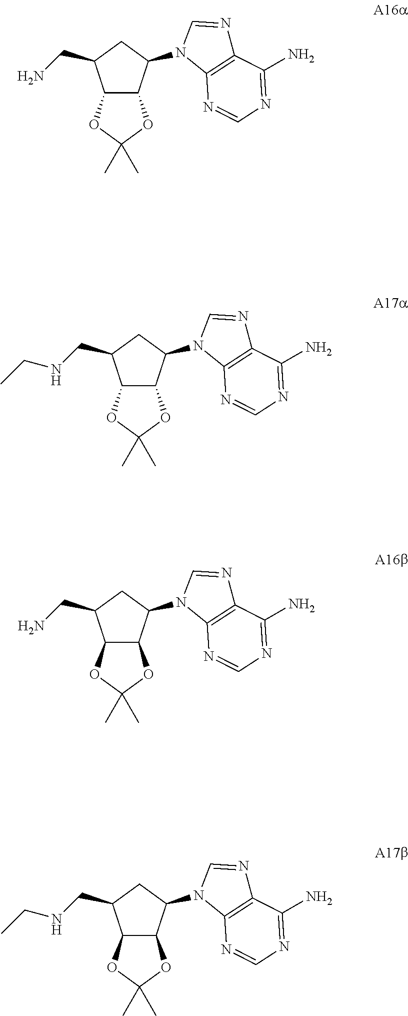 [SMILES: CC1(C)O[C@@H]2[C@@H](CN)C[C@@H](n3cnc4c(N)ncnc43)[C@@H]2O1 ; CCNC[C@H]1C[C@@H](n2cnc3c(N)ncnc32)[C@@H]2OC(C)(C)O[C@H]12 ; CC1(C)O[C@H]2[C@@H](CN)C[C@@H](n3cnc4c(N)ncnc43)[C@H]2O1 ; CCNC[C@H]1C[C@@H](n2cnc3c(N)ncnc32)[C@H]2OC(C)(C)O[C@@H]12]

9-((3aS,4R,6R,6aR)-6-(Aminomethyl)-2,2-dimethyltetrahydro-4H-cyclopenta[d][1,3]dioxol-4-yl)-9H-purin-6-amine (A16α) and 9-((3aS,4R,6R,6aR)-6-((Ethylamino)methyl)-2,2-dimethyltetrahydro-4H-cyclopenta[d][1,3]dioxol-4-yl)-9H-purin-6-amine (A17α) and 9-((3aS,4R,6R,6aR)-6-(Aminomethyl)-2,2-dimethyltetrahydro-4H-cyclopenta[d][1,3]dioxol-4-yl)-9H-purin-6-amine (A16β) and 9-((3aS,4R,6R,6aR)-6-((Ethylamino)methyl)-2,2-dimethyltetrahydro-4H-cyclopenta[d][1,3]dioxol-4-yl)-9H-purin-6-amine (A17β). To a solution of diastereomeric alcohols A14α/A14β (0.3 g, 0.98 mmol, 1.0 eq.) in 1,4-dioxane (25 mL) under an atmosphere of argon was added diphenylphosphoryl azide (0.43 mL, 1.96 mmol, 2.0 eq.) and DBU (0.44 mL, 2.94 mmol, 3.0 eq.), and the reaction mixture was stirred for 18 h at room temperature. Sodium azide (0.32 g, 4.91 mmol, 5.0 eq.) and 15-crown-5 (1.9 μL, 0.01 mmol, 0.01 eq.) were added, and the reaction mixture was heated at 110° C. for 4 h. The solid was removed by filtration and the solvent was removed under vacuum. The crude product was directly used in the next reaction. To a solution of diastereomeric azides A15α/A15β (0.23 g, 0.69 mmol) in ethanol (20 mL) was added 10% Pd/C (0.046 g, 20% w/w) and the mixture stirred under an atmosphere of hydrogen for 48 h. The mixture was filtered through Celite and washed with methanol and the solvent removed under vacuum. The crude product was purified by column chromatography (60-70% MeOH in CH₂Cl₂) to give two separable fractions. The first fraction contained an inseparable 1:2 mixture of A16α and A17α (50 mg, 24%) and the second fraction contained an inseparable 2:1 mixture of A16β and A17β (58 mg, 25%). Compound A16α: ¹H NMR (600 MHz, C_{D}-OD) δ 8.20 (s, 2H), 5.22-5.01 (m, 1H), 4.89 (dt, J=13.3, 6.9 Hz, 1H), 4.59 (dd, J=7.3, 5.5 Hz, 1H), 2.88 (dd, J=12.7, 6.4 Hz, 1H), 2.78 (dd, J=12.6, 6.2 Hz, 1H), 2.74-2.70 (m, 2H), 2.48 (dt, J=12.7, 6.7 Hz, 1H), 1.55 (s, 3H), 1.31 (s, 3H).

Compound A17α: ¹H NMR (600 MHz, CD₃OD) δ 8.22 (d, J=2.4 Hz, 2H), 5.18-5.05 (m, 2H), 4.89 (dt, J=13.3, 6.9 Hz, 1H), 4.59 (dd, J=7.3, 5.5 Hz, 1H), 2.83 (dd, J=11.9, 7.4 Hz, 1H), 2.70-2.66 (m, 2H) 2.48 (dt, J=12.7, 6.7 Hz, 1H), 2.38 (dt, J=12.8, 6.4 Hz, 1H), 2.26 (q, J=12.2 Hz, 1H), 1.55 (s, 3H), 1.31 (s, 3H), 1.14 (t, J=7.2 Hz, 3H).

Compound A16β: ¹H NMR (600 MHz, CD₃OD) δ 8.21 (d, J=2.5 Hz, 2H), 4.81-4.76 (m, 3H), 2.95 (dd, J=12.8, 6.1 Hz, 1H), 2.84 (dd, J=12.8, 6.1 Hz, 1H), 2.32-2.06 (m, 3H), 1.47 (s, 3H), 1.26 (s, 3H).

Compound A17β: ¹H NMR (600 MHz, CD₃OD) δ 8.21 (d, J=2.5 Hz, 2H), 4.81-4.76 (m, 3H), 2.92 (dd, J=12.8, 5.5 Hz, 1H), 2.77 (dd, J=12.8, 5.5 Hz, 1H), 2.68 (q, J=7.2 Hz, 2H), 2.32-2.06 (m, 3H), 1.47 (s, 3H), 1.26 (s, 3H), 1.15 (t, J=7.2 Hz, 3H).

Compound A16α and A16β: HRMS (ESI⁺): m/z calcd for C₁₄H₂₁N₆O₂ [M+H]⁺ 305.1721, found 305.1721.

Compound A17α and A17β: HRMS (ESI⁺): m/z calcd for C₁₆H₂₅N₆O₂ [M+H]⁺ 333.2034, found 333.2034.

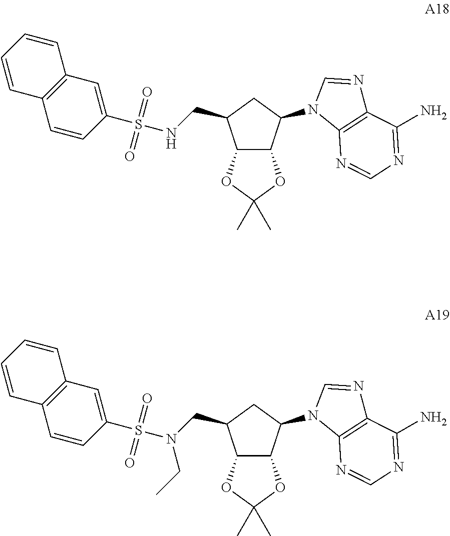 [SMILES: CC1(C)O[C@@H]2[C@@H](CNS(=O)(=O)c3ccc4ccccc4c3)C[C@@H](n3cnc4c(N)ncnc43)[C@@H]2O1 ; CCN(C[C@H]1C[C@@H](n2cnc3c(N)ncnc32)[C@@H]2OC(C)(C)O[C@H]12)S(=O)(=O)c1ccc2ccccc2c1]

N-(((3aR,4R,6R,6aS)-6-(6-Amino-9H-purin-9-yl)-2,2-dimethyltetrahydro-4H-cyclopenta[d][1,3]dioxol-4-yl)methyl)naphthalene-2-sulfonamide (A18) and N-(((3aR,4R,6R,6aS)-6-(6-Amino-9H-purin-9-yl)-2,2-dimethyltetrahydro-4H-cyclopenta[d][1,3]dioxol-4-yl)methyl)-N-ethylnaphthalene-2-sulfonamide (A19). To mixture of compounds A16α and A17α (50 mg, 0.16 mmol, 1.0 eq.) in CH₂Cl₂ (10 mL) under argon atmosphere at room temperature was added triethylamine (0.07 mL, 0.49 mmol, 3.0 eq.). The mixture was cooled to 0° C. and 2-naphthalenesulfonyl chloride (55 mg, 0.25 mmol, 1.5 eq.) was added. After the reaction mixture was stirred for 24 h at room temperature, water (20 mL) was added, and the aqueous layer was extracted with CH₂Cl₂. The combined organic layer was washed with brine and dried over Na₂SO₄. After filtration, the solvent was removed under vacuum and the crude product was purified by column chromatography (10-13% MeOH in CH₂Cl₂) to give two separable compounds A18 (22 mg, 27%) and compound A19 (33 mg, 39%).

Compound A18:¹H NMR (600 MHz, CD₃OD) δ 8.44 (d, J=1.9 Hz, 1H), 8.16 (s, 1H), 8.14 (s, 1H), 8.05-8.00 (m, 2H), 7.95 (d, J=8.1 Hz, 1H), 7.86 (dd, J=8.8, 1.9 Hz, 1H), 7.75-7.60 (m, 2H), 5.01 (t, J=6.6 Hz, 1H), 4.78 (dt, J=12.5, 6.4 Hz, 1H), 4.52 (dd, J=7.3, 5.0 Hz, 1H), 3.15 (dd, J=13.4, 6.3 Hz, 1H), 3.07 (dd, J=13.2, 6.8 Hz, 1H), 2.36 (dt, J=12.0, 6.9 Hz, 1H), 2.33-2.26 (m, 1H), 2.19-2.10 (m, 1H), 1.46 (s, 3H), 1.21 (s, 3H). HRMS (ESI⁺): m/z calcd for C₂₄H₂₇N₆O₄S [M+H]⁺ 495.1809, found 495.1808.

Compound A19:¹H NMR (400 MHz, CD₃OD) δ 8.42 (d, J=2.0 Hz, 1H), 8.17 (d, J=5.6 Hz, 2H), 8.01 (d, J=8.5 Hz, 2H), 7.93 (dd, J=7.7, 1.7 Hz, 1H), 7.81 (dd, J=8.7, 1.9 Hz, 1H), 7.73-7.56 (m, 2H), 5.09 (dd, J=7.3, 5.8 Hz, 1H), 4.80 (d, J=6.3 Hz, 1H), 4.62 (dd, J=7.3, 5.0 Hz, 1H), 3.50 (dd, J=14.0, 7.9 Hz, 1H), 3.44-3.34 (m, 2H), 3.29 (dd, J=14.0, 7.9 Hz, 1H), 2.50 (td, J=7.1, 3.6 Hz, 1H), 2.38 (dt, J=13.4, 6.8 Hz, 1H), 2.18 (q, J=11.9 Hz, 1H), 1.50 (s, 3H), 1.23 (s, 3H), 1.13 (t, J=7.1 Hz, 3H). ¹³C NMR (100 MHz, CD₃OD) δ 157.3, 153.5, 150.6, 141.6, 138.2, 136.2, 133.6, 130.6, 130.2, 129.9, 129.4, 128.9, 128.7, 123.6, 120.6, 115.0, 84.7, 83.6, 62.7, 50.9, 44.2, 44.1, 36.5, 27.7, 25.3, 14.1. HRMS (ESI⁺): m/z calcd for C₂₆H₃₁N₆O₄S [M+H]⁺ 523.2122, found 523.2122.

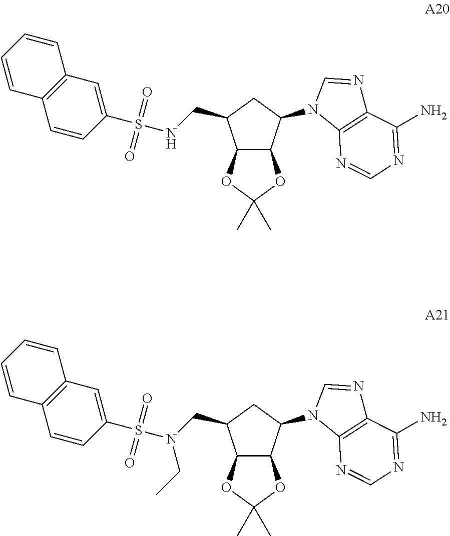 [SMILES: CC1(C)O[C@H]2[C@@H](CNS(=O)(=O)c3ccc4ccccc4c3)C[C@@H](n3cnc4c(N)ncnc43)[C@H]2O1 ; CCN(C[C@H]1C[C@@H](n2cnc3c(N)ncnc32)[C@H]2OC(C)(C)O[C@@H]12)S(=O)(=O)c1ccc2ccccc2c1]

N-(((3aS,4R,6R,6aR)-6-(6-Amino-9H-purin-9-yl)-2,2-dimethyltetrahydro-4H-cyclopenta[di][1,3]dioxol-4-yl)methyl)naphthalene-2-sulfonamide (A20) and N-(((3aS,4R,6R,6aR)-6-(6-Amino-9H-purin-9-yl)-2,2-dimethyltetrahydro-4H-cyclopenta[d][1,3]dioxol-4-yl)methyl)-N-ethylnaphthalene-2-sulfonamide (A21). To mixture of compound A16β and A17β (58 mg, 0.19 mmol, 1.0 eq.) in CH₂Cl₂ (30 mL) under argon atmosphere at room temperature was added triethylamine (0.08 mL, 0.57 mmol, 3.0 eq.). The mixture was cooled to 0° C. and 2-naphthalenesulfonyl chloride (65 mg, 0.29 mmol, 1.5 eq.) was added. After the reaction mixture was stirred for 24 h at room temperature, water (20 mL) was added, and the aqueous layer was extracted with CH₂Cl₂. The combined organic layer was washed with brine and dried over Na₂SO₄. After filtration, the solvent was removed under vacuum and the crude product was purified by column chromatography (10-13% MeOH in CH₂Cl₂) to give two separable compounds A20 (40 mg, 49%) and compound A21 (33 mg, 24%).

Compound A20: ¹H NMR (400 MHz, CD₃OD) δ 8.46 (d, J=1.9 Hz, 1H), 8.18 (s, 1H), 8.07 (d, J=8.8 Hz, 2H), 8.02 (s, 1H), 8.01-7.97 (m, 1H), 7.88 (dd, J=8.7, 1.9 Hz, 1H), 7.70-7.64 (m, 2H), 4.76-4.67 (m, 2H), 4.63 (t, J=4.9 Hz, 1H), 3.20 (dd, J=13.0, 7.4 Hz, 1H), 3.13 (dd, J=12.9, 7.0 Hz, 1H), 2.26-2.11 (m, 1H), 2.12-1.99 (m, 2H), 1.32 (s, 3H), 1.13 (s, 3H). HRMS (ESI): m/z calcd for C₂₄H₂₇N₆O₄S [M+H]⁺ 495.1809, found 495.1807.

Compound A21: ¹H NMR (600 MHz, CDCl₃) δ 8.42 (d, J=1.8 Hz, 1H), 8.34 (s, 1H), 7.97 (t, J=9.0 Hz, 3H), 7.92 (d, J=9.1 Hz, 3H), 7.81 (dd, J=8.6, 1.9 Hz, 1H), 7.73-7.59 (m, 2H), 4.82 (dt, J=13.0, 5.5 Hz, 1H), 4.74 (t, J=5.2 Hz, 1H), 4.67 (t, J=5.2 Hz, 1H), 3.56 (dd, J=14.5, 7.9 Hz, 1H), 3.37-3.34 (m, 2H), 3.31-3.27 (m, 1H). 2.36 (td, J=10.9, 8.9, 4.2 Hz, 1H), 2.21 (dt, J=11.4, 5.8 Hz, 1H), 2.07 (q, J=12.6 Hz, 1H), 1.48 (s, 3H), 1.24 (s, 3H), 1.13 (t, J=7.1 Hz, 3H). HRMS (ESI⁺): m/z calcd for C₂₆H₃₁N₆O₄S [M+H]⁺ 523.2122, found 523.2122.

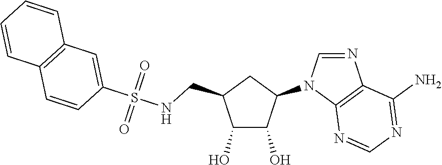 [SMILES: Nc1ncnc2c1ncn2[C@@H]1C[C@H](CNS(=O)(=O)c2ccc3ccccc3c2)[C@@H](O)[C@H]1O]

N-(((1R,2R,3S,4R)-4-(6-Amino-9H-purin-9-yl)-2,3-dihydroxycyclopentyl)methyl)naphthalene-2-sulfonamide (A1). To compound A18 (22 mg, 0.04 mmol) was added a solution of TFA/H₂O (1.25 mL, 4:1) at 0° C. The reaction mixture was stirred at 0° C. for 30 min, allowed to warm to room temperature and stirred for additional 3 h. The reaction mixture was diluted with methanol (5 mL) and the solvents were removed under vacuum. The crude product was purified by column chromatography (10% MeOH in CH₂Cl₂) to give compound A1 as a white solid (8 mg, 40%). ¹H NMR (400 MHz, CD₃OD) δ 8.45 (d, J=1.9 Hz, 1H), 8.15 (d, J=7.2 Hz, 2H), 8.08-8.01 (m, 2H), 7.96 (dd, J=8.1, 1.2 Hz, 1H), 7.86 (dd, J=8.7, 1.9 Hz, 1H), 7.68-7.61 (m, 2H), 4.75 (dt, J=10.3, 8.6 Hz, 1H), 4.51 (dd, J=8.6, 5.5 Hz, 1H), 3.98 (dd, J=5.5, 3.5 Hz, 1H), 3.16 (dd, J=13.0, 6.4 Hz, 1H), 3.06 (dd, J=13.0, 7.0 Hz, 1H), 2.39 (dt, J=13.2, 8.6 Hz, 1H), 2.27-2.18 (m, 1H), 1.82 (ddd, J=13.2, 10.3, 8.2 Hz, 1H). ¹³C NMR (100 MHz, CD₃OD) δ 156.8, 152.6, 150.8, 142.3, 138.7, 136.2, 133.6, 130.6, 130.2, 129.8, 129.1, 129.0, 128.7, 123.4, 120.7, 76.11, 74.2, 62.1, 46.9, 44.7, 31.1. HRMS (ESI⁺): m/z calcd for C₂₁H₂₃N₆O₄S [M+H]⁺ 455.1496, found 455.1495.

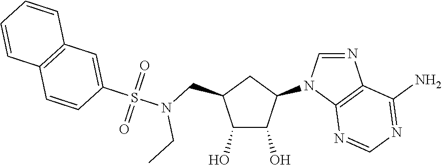 [SMILES: CCN(C[C@H]1C[C@@H](n2cnc3c(N)ncnc32)[C@H](O)[C@@H]1O)S(=O)(=O)c1ccc2ccccc2c1]

N-(((1R,2R,3S,4R)-4-(6-Amino-9H-purin-9-yl)-2,3-dihydroxycyclopentyl)methyl)-N-ethylnaphthalene-2-sulfonamide (A2). To compound A19 (33 mg, 0.07 mmol) was added a solution of TFA/H₂O (2.5 mL, 4:1) at 0° C. The reaction mixture was stirred at 0° C. for 30 min, allowed to warm to room temperature and stirred for additional 3 h. The reaction mixture was diluted with methanol (5 mL) and the solvents were removed under vacuum. The crude product was purified by column chromatography (10% MeOH in CH₂Cl₂) to give compound A2 as a white solid (16 mg, 53%). ¹H NMR (400 MHz, CD₃OD) δ 8.44 (d, J=1.9 Hz, 1H), 8.20 (d, J=3.5 Hz, 2H), 8.11-8.01 (m, 2H), 7.96 (dd, J=8.1, 1.2 Hz, 1H), 7.84 (dd, J=8.7, 1.9 Hz, 1H), 7.72-7.57 (m, 2H), 4.82-4.76 (m, 1H), 4.61 (dd, J=8.7, 5.4 Hz, 1H), 4.11 (dd, J=5.5, 2.8 Hz, 1H), 3.61 (dd, J=14.0, 7.5 Hz, 1H), 3.45-3.35 (m, 2H), 3.26 (dd, J=14.0, 7.5 Hz, 1H), 2.46-2.37 (m, 2H), 1.85 (dd, J=10.3, 5.4 Hz, 1H), 1.14 (t, J=7.1 Hz, 3H). ¹³C NMR (100 MHz, CD₃OD) S 156.5, 152.2, 150.9, 142.5, 138.2, 136.2, 133.7, 130.6, 130.2, 129.9, 129.4, 129.0, 128.8, 124.6, 120.7, 75.9, 74.0, 62.0, 52.0, 44.3, 43.4, 31.5, 14.4. HRMS (ESI⁺): m/z calcd for C₂₃H₂₇N₆O₄S [M+H]⁺ 483.1809, found 483.1807.

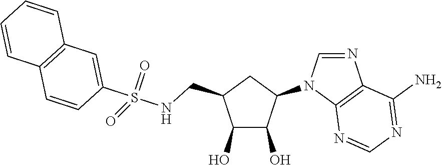 [SMILES: Nc1ncnc2c1ncn2[C@@H]1C[C@H](CNS(=O)(=O)c2ccc3ccccc3c2)[C@H](O)[C@@H]1O]

N-(((1R,2S,3R,4R)-4-(6-Amino-9H-purin-9-yl)-2,3-dihydroxycyclopentyl)methyl)naphthalene-2-sulfonamide (A3). To compound A20 (40 mg, 0.08 mmol) was added a solution of TFA/H₂O (2.5 mL, 4:1) at 0° C. The reaction mixture was stirred at 0° C. for 30 min, allowed to warm to room temperature and stirred for additional 3 h. The reaction mixture was diluted with methanol (5 mL) and the solvents were removed under vacuum. The crude product was purified by column chromatography (20% MeOH in CH₂Cl₂) to give compound A3 as a white solid (12 mg, 33%). ¹H NMR (400 MHz, CD₃OD) δ 8.45 (d, J=1.8 Hz, 1H), 8.21 (s, 1H), 8.16 (s, 1H), 8.10-8.01 (m, 2H), 7.97 (dd, J=8.2, 1.5 Hz, 1H), 7.87 (dd, J=8.7, 1.9 Hz, 1H), 7.72-7.57 (m, 2H), 4.98 (td, J=8.9, 7.3 Hz, 1H), 4.21 (dd, J=7.4, 4.1 Hz, 1H), 4.13 (dd, J=5.3, 4.1 Hz, 1H), 3.25 (dd, J=12.9, 6.7 Hz, 1H), 3.11 (dd, J=12.9, 7.5 Hz, 1H), 2.37-2.29 (m, 1H), 2.26-2.15 (m, 1H), 1.96 (ddd, J=12.8, 11.0, 9.3 Hz, 1H). ¹³C NMR (100 MHz, CD₃OD) δ 156.9, 152.8, 143.6, 138.8, 136.2, 133.7, 130.5, 130.2, 129.8, 129.1, 129.0, 128.7, 123.5, 120.7, 119.6, 73.7, 73.5, 55.6, 44.3, 41.0, 34.5. HRMS (ESI⁺): m/z calcd for C₂₁H₂₃N₆O₄S [M+H]⁺ 455.1496, found 455.1495.

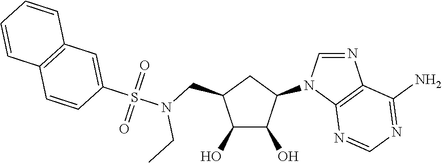 [SMILES: CCN(C[C@H]1C[C@@H](n2cnc3c(N)ncnc32)[C@@H](O)[C@H]1O)S(=O)(=O)c1ccc2ccccc2c1]

N-(((1R,2S,3R,4R)-4-(6-Amino-9H-purin-9-yl)-2,3-dihydroxycyclopentyl)methyl)-N-ethylnaphthalene-2-sulfonamide (A4). To compound A21 (20 mg, 0.04 mmol) was added a solution of TFA/H₂O (1.5 mL, 4:1) at 0° C. The reaction mixture was stirred at 0° C. for 30 min, allowed to warm to room temperature and stirred for additional 3 h. The reaction mixture was diluted with methanol (5 mL) and the solvents were removed under vacuum. The crude product was purified by column chromatography (15% MeOH in CH₂Cl₂) to give compound A4 as a white solid (6 mg, 33%). 4H NMR (400 MHz, CD₃OD) δ 8.45 (d, J=1.8 Hz, 1H), 8.31 (s, 1H), 8.20 (s, 1H), 8.10-7.99 (m, 2H), 7.96 (d, J=8.0 Hz, 1H), 7.85 (dd, J=8.6, 1.9 Hz, 1H), 7.75-7.60 (m, 2H), 5.09 (q, J=8.3 Hz, 1H), 4.29 (dd, J=8.0, 3.9 Hz, 1H), 4.15 (d, J=4.1 Hz, 1H), 3.62 (dd, J=14.2, 7.9 Hz, 1H), 3.59-3.36 (m, 3H), 2.39 (p, J=7.6 Hz, 2H), 2.17-1.95 (m, 1H), 1.13 (t, J=7.1 Hz, 3H). ¹³C NMR (100 MHz, CD₃OD) δ 156.5, 152.2, 150.1, 144.1, 138.3, 136.2, 133.7, 130.6, 130.2, 129.9, 129.4, 128.9, 128.7, 123.6, 119.6, 73.9, 73.7, 55.5, 48.9, 44.5, 40.3, 35.1, 14.4. HRMS (ESI⁺): m/z calcd for C₂₃H₂₇N₆O₄S [M+H]⁺ 483.1809, found 483.1809.

### REFERENCES FOR EXAMPLE 2

(1) Bockman, M. R.; Kalinda, A. S.; Petrelli, R.; De la Mora-Rey.; Tiwari, D.; Feng Liu, F.; Dawadi, S.; Nandakumar, M.; Rhee, K. Y.; Schnappinger, D.; Finzel, B. C.; Aldrich. C. C. J. Med. Chem. 2015, 58, 7349-7369.
(2) Xu, C.-P.; Xiao, Z.-H.; Zhuo, B.-Q.; Wang, Y.-H.; Huang, P.-Q. Chem. Commun. 2010, 46, (41), 7834-7836.

### Example 3

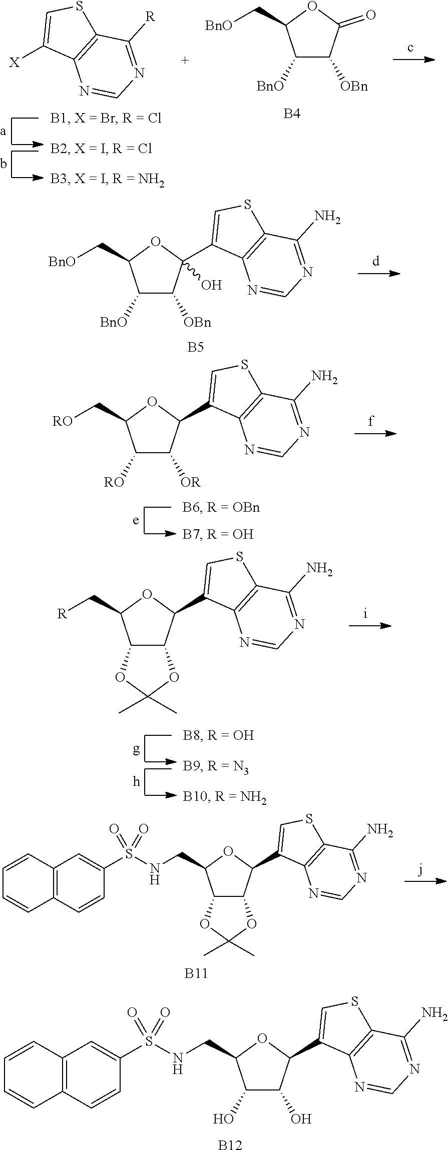

Scheme 6. Reagents and conditions: (a) i. ⁱPrMgCl, THF, 0° C.; ii. I₂, 0° C.; (b) aqueous ammonia, 1,4-dioxane, 60° C.; (c) i. TMSCl, PhMgCl, THF, rt to 0° C.; ii. ⁱPrMgCl·LiCl, THF, −20° C.; (d) BF₃·OEt₂, Et₃SiH, CH₂Cl_{2, 0}° C.; (e) BCl₃, CH₂Cl_{2, -78}° C.; (f) 2,2-dimethoxypropane, pTsOH, acetone, rt; (g) i. DPPA, DBU, 1,4-dioxane, rt; ii. NaN₃, TBAI, 15-crown-5, reflux; (h) H₂, Pd/C, MeOH; (i) 2-naphthalenesulfonyl chloride, Et₃N, DMAP, CH₂Cl₂, rt; (j) TFA/H₂O (4:1), 0° C. to rt.

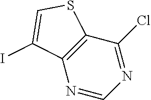 [SMILES: Clc1ncnc2c(I)csc12]

4-Chloro-7-iodothieno[3,2-d]pyrimidine (B2). To a suspension of 7-bromo-4-chlorothieno[3,2-d]pyrimidine (1B1, 6.58 g, 26.4 mmol) in anhydrous THF (160 mL) at 0° C. was added ⁱPrMgCl (2.0 M in THF, 19.8 mL, 39.6 mmol) dropwise via an additional funnel. The resulting clear mixture was allowed to stir at 0° C. for 1 h and iodine (10.0 g, 39.4 mmol) in anhydrous THE (70 mL) was added dropwise via an additional funnel. After the resulting mixture was allowed to stir at 0° C. for 2 h, the reaction mixture was poured in to a saturated NH₄Cl solution (330 mL) and THE was removed in vacuo. After addition of EtOAc (500 mL) and water (100 mL), the organic layer was separated, washed with saturated Na₂SO₃ (330 mL) and dried over Na₂SO₄. After filtration, the filtrate was concentrated, and the resulting dark tan solid was treated with a mixture of MeOH (80 mL) and water (10 mL). After the mixture was allowed to stir vigorously for 30 min, the solid was filtered, washed with MeOH, suction-dried, and dried in vacuo to give compound B2 as a tan solid (3.67 g, 47%). ¹H NMR (400 MHz, DMSO-d6) δ 9.14 (s, 1H), 8.82 (s, 1H). ¹³C NMR (100 MHz, DMSO-d6) δ 161.2, 154.9, 153.7, 140.6, 129.1, 83.0.

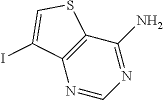 [SMILES: Nc1ncnc2c(I)csc12]

7-Iodothieno[3,2-d]pyrimidin-4-amine (B3). A suspension of compound B2 in 1,4-dioxane (130 mL) and strong aqueous ammonia (130 mL) in a sealed tube was heated at 60° C. for 70 h. After being allowed to cool to rt, the reaction mixture was concentrated to a volume of about 50 mL. The solid formed was filtered, washed with water (about 20 mL), suction-dried, and dried in vacuo to give compound B3 as a tan solid (3.11 g, 91%). ¹H NMR (400 MHz, DMSO-d6) δ 8.44 (s, 1H), 8.38 (s, 1H), 7.60 (brs, 2H). ¹³C NMR (100 MHz, DMSO-d6) δ 158.8, 158.3, 155.5, 134.8, 112.7, 82.7. HRMS (ESI): m/z calcd for C₆H₅IN₃S [M+H]⁺ 277.9243, found 277.9243.

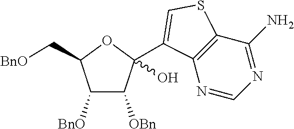 [SMILES: Nc1ncnc2c(C3(O)O[C@H](COCc4ccccc4)[C@@H](OCc4ccccc4)[C@H]3OCc3ccccc3)csc12]

(3R,4R,5R)-2-(4-Aminothieno[3,2-d]pyrimidin-7-yl)-3,4-bis(benzyloxy)-5-((benzyloxy)methyl)tetrahydrofuran-2-ol (B5). To a solution of compound B3 (1.66 g, 5.99 mmol) in anhydrous THE (30 mL) was added TMSCl (1.53 mL, 12.0 mmol) and the resulting mixture was allowed to stir at rt for 20 min. The mixture was then cooled to 0° C., and PhMgCl (2.0 M in THE, 6.0 mL, 12.0 mmol) was added dropwise. The reaction mixture was allowed to stir for 20 min and ⁱPrMgCl·LiCl (1.3 M in THF, 4.7 mL, 6.11 mmol) was added. After 40 min, the reaction mixture was cooled to approximately −20° C. and a solution of 2,3,5-tri-O-benzyl-D-ribono-1,4-lactone (B4, 2.52 g, 6.02 mmol) in anhydrous THE (6+2 mL) was added dropwise. The reaction mixture was allowed to stir at approximately −20° C. overnight and was then quenched sequentially with methanol (6 mL), acetic acid (6 mL) and water (6 mL). After being allowed to warm to rt, the reaction mixture was diluted with EtOAc (150 mL), and the resulting mixture was washed with 1 N HCl (30 mL), saturated NaHCO₃ (30 mL), and brine (150 mL). The organic layer was dried over Na₂SO₄ and filtered. The filtrate was concentrated, and the residue was purified by flash column chromatography (50% to 100% EtOAc/hexanes) to give compound B5 as a yellowish foam solid (1.09 g, 32%). HRMS (APCI⁺): m/z calcd for C₃₂H₃₂N₃O₅S [M+H]⁺ 570.2057, found 570.2079.

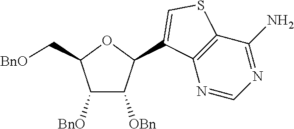 [SMILES: Nc1ncnc2c([C@@H]3O[C@H](COCc4ccccc4)[C@@H](OCc4ccccc4)[C@H]3OCc3ccccc3)csc12]

7-((2S,3S,4R,5R)-3,4-Bis(benzyloxy)-5-((benzyloxy)methyl)tetrahydrofuran-2-yl)thieno[3,2-d]pyrimidin-4-amine (B6). To a solution of compound B5 (1.08 g, 1.90 mmol) in anhydrous CH₂Cl₂ (20 mL) at 0° C. was added triethylsilane (1.52 mL, 9.52 mmol), followed by slow addition of BF₃·OEt₂ (0.71 mL, 5.75 mmol). The resulting solution was allowed to stir at 0° C. for 3 h. Additional triethylsilane (0.92 mL, 5.76 mmol) and BF₃·OEt₂ (0.48 mL, 3.89 mmol) were added. The reaction mixture was allowed to stir at 0° C. for additional 7 h and placed in a refrigerator overnight. The mixture was cooled at 0° C. and quenched with saturated NaHCO₃ (30 mL). After separation, the aqueous layer was extracted with CH₂Cl₂ (20 mL). The combined organic layer was washed with brine (40 mL) and dried over Na₂SO₄. After filtration, the filtrate was concentrated, and the residue was purified by flash column chromatography (0% to 10% MeOH/CH₂Cl₂) to give compound B6 as a pale solid (892 mg, 85%). ¹H NMR (400 MHz, CDCl₃) δ 8.63 (s, 1H), 7.93 (d, J=1.2 Hz, 1H), 7.48-7.42 (m, 2H), 7.38-7.17 (m, 13H), 5.66 (t, J=1.4 Hz, 1H), 5.25 (brs, 2H), 4.96 (s, 2H), 4.62 (d, J=11.7 Hz, 1H), 4.54 (d, J=11.8 Hz, 1H), 4.46-4.37 (m, 2H), 4.26 (d, J=11.9 Hz, 1H), 4.22 (dd, J=4.6, 1.8 Hz, 1H), 4.12 (dd, J=8.4, 4.6 Hz, 1H), 3.96 (dd, J=10.8, 2.6 Hz, 1H), 3.72 (dd, J=10.8, 3.2 Hz, 1H). ¹³C NMR (100 MHz, CDCl₃) δ 158.1, 157.9, 154.8, 138.5, 138.4, 138.1, 137.4, 129.6, 128.7 (×2), 128.6 (×2), 128.5 (×2), 128.4 (×2), 127.9, 127.9 (×2), 127.8 (×2), 127.8, 115.8, 80.1, 79.9, 79.4, 76.4, 73.6, 72.1, 71.3, 69.2. HRMS (ESI⁺): m/z calcd for C₃₂H₃₂N₃O₄S [M+H]⁺ 554.2108, found 554.2109.

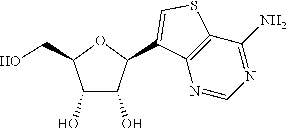 [SMILES: Nc1ncnc2c([C@@H]3O[C@H](CO)[C@@H](O)[C@H]3O)csc12]

(2S,3R,4S,5R)-2-(4-Aminothieno[3,2-d]pyrimidin-7-yl)-5-(hydroxymethyl)tetrahydrofuran-3,4-diol (B7). To a solution of compound B6 (734 mg, 1.32 mmol) in anhydrous CH₂Cl₂ (13 mL) at −78° C., was added BCl₃ (1.0 M in CH₂Cl_{2, 6.0} mL, 6.0 mmol). The resulting mixture was allowed to stir at −78° C. for 3.5 h. The reaction was quenched with slow addition of methanol (4 mL), allowed to warn to rt, and concentrated. The residue was co-evaporated with MeOH (26×3 mL) and then dissolved in MeOH (13 mL). The mixture was neutralized with strong aqueous ammonia and concentrated. The residue was purified by flash column chromatography (0% to 20% MeOH/CH₂Cl₂) to give compound B7 as a white foam solid (306 mg, 82%). ¹H NMR (400 MHz, CD₃OD) δ 8.34 (s, 1H), 8.00 (d, J=0.4 Hz, 1H), 5.07 (d, J=7.6 Hz, 1H), 4.48 (dd, J=7.6, 5.2 Hz, 1H), 4.22 (dd, J=5.1, 2.6 Hz, 1H), 4.12 (q, J=2.6 Hz, 1H), 3.86 (dd, J=12.3, 2.5 Hz, 1H), 3.72 (dd, J=12.3, 2.6 Hz, 1H). ¹³C NMR (100 MHz, CD₃OD) δ 160.6, 157.9, 155.0, 136.8, 132.7, 117.5, 87.7, 82.0, 76.6, 74.1, 64.2. HRMS (ESI): m/z calcd for C₁₁H₁₄N₃O₄S [M+H]⁺ 284.0700, found 284.0698.

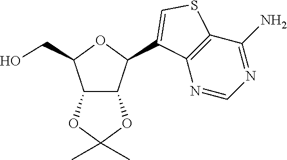 [SMILES: CC1(C)O[C@@H]2[C@H](O1)[C@@H](CO)O[C@H]2c1csc2c(N)ncnc12]

((3aR,4R,6S,6aS)-6-(4-Aminothieno[3,2-d]pyrimidin-7-yl)-2,2-dimethyltetrahydrofuro[3,4-d][1,3]dioxol-4-yl)methanol (B8). To a solution of compound B7 (340 mg, 1.20 mmol) in acetone (12 mL) were added 2,2-dimethoxypropane (0.82 mL, 6.61 mmol) and pTsOH (253 mg, 1.33 mmol). The resulting mixture was allowed to stir overnight and then quenched with NaHCO₃ (134 mg, 1.60 mmol) and water (1.4 mL). After 30 min, the reaction mixture was concentrated, and the residue was purified by flash column chromatography (0% to 10% MeOH/CH₂Cl₂) to give compound B8 as a pale solid (333 mg, 86%). ¹H NMR (400 MHz, CD₃OD) δ 8.35 (s, 1H), 8.03 (d, J=0.4 Hz, 1H), 5.16 (d, J=5.4 Hz, 1H), 4.94 (t, J=5.8 Hz, 1H), 4.90 (dd, J=6.2, 2.5 Hz, 1H), 4.29 (q, J=2.8 Hz, 1H), 3.83 (dd, J=12.1, 2.8 Hz, 1H), 3.73 (dd, J=12.1, 3.1 Hz, 1H), 1.61 (s, 3H), 1.35 (s, 3H). BC NMR (100 MHz, CD₃OD) δ 160.5, 158.0, 155.3, 136.2, 132.8, 117.5, 115.3, 86.2, 85.9, 84.2, 84.1, 64.2, 28.2, 25.9. HRMS (ESI⁺): m/z calcd for C₁₄H₁₈N₃O₄S [M+H]⁺ 324.1013, found 324.1012.

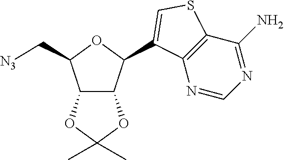 [SMILES: CC1(C)O[C@@H]2[C@H](O1)[C@@H](CN=[N+]=[N-])O[C@H]2c1csc2c(N)ncnc12]

7-((3aS,4S,6R,6aR)-6-(Azidomethyl)-2,2-dimethyltetrahydrofuro[3,4-d][1,3]dioxol-4-yl)thieno[3,2-d]pyrimidin-4-amine (B9). To a solution of compound B8 (320 mg, 0.990 mmol) in anhydrous 1,4-dioxane (6 mL) were added diphenylphosphoryl azide (0.43 mL, 1.99 mmol) and DBU (0.45 mL, 2.98 mmol). After the resulting mixture was allowed to stir at rt for 4.5 h, sodium azide (323 mg, 4.97 mmol), tetra-n-butylammonium iodide (37 mg, 0.10 mmol) and 15-crown-5 (20 μL, 0.10 mmol) were added, and the reaction mixture was heated at reflux for 5 h. After being allowed to cool to rt, the mixture was diluted with 5% MeOH/EtOAc (18 mL), filtered through a pad of Celite, and washed with 5% MeOH/EtOAc (54 mL). The filtrate was washed with water (18×2 mL) and brine (80 mL). The organic layer was dried over Na₂SO₄ and filtered. The filtrate was concentrated, and the residue was purified by flash column chromatography (0% to 10% MeOH/CH₂Cl₂) to give compound B9 as a yellowish solid (107 mg, 31%). ¹H NMR (400 MHz, CD₃OD) δ 8.41 (s, 1H), 8.02 (d, J=0.6 Hz, 1H), 5.32 (d, J=4.2 Hz, 1H), 5.02 (dd, J=6.6, 4.2 Hz, 1H), 4.78 (dd, J=6.6, 4.6 Hz, 1H), 4.18 (ddd, J=5.6, 4.4, 4.4 Hz, 1H), 3.58 (dd, J=13.2, 4.1 Hz, 1H), 3.49 (dd, J=13.2, 5.7 Hz, 1H), 1.59 (s, 3H), 1.35 (s, 3H). ¹³C NMR (100 MHz, CD₃OD) δ 160.3, 158.6, 155.7, 136.4, 132.0, 117.1, 116.1, 86.5, 85.0, 83.8, 82.2, 53.5, 27.9, 25.9. HRMS (ESI⁺): m/z calcd C₁₄H₁₇N₆O₃S [M+H]⁺ 349.1077, found 349.1078.

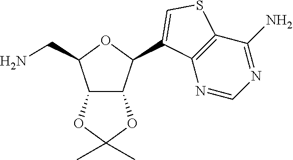 [SMILES: CC1(C)O[C@@H]2[C@H](O1)[C@@H](CN)O[C@H]2c1csc2c(N)ncnc12]

7-((3aS,4S,6R,6aR)-6-(Aminomethyl)-2,2-dimethyltetrahydrofuro[3,4-d][1,3]dioxol-4-yl)thieno[3,2-d]pyrimidin-4-amine (B10). To a solution of compound B9 (103 mg, 0.296 mmol) in MeOH (3 mL) was added 10% Pd/C (21 mg, 20% w/w), and the mixture was allowed to stir under an atmosphere of hydrogen for 15 h. The mixture was filtered through a pad of Celite and the filtrate was concentrated. The residue was purified by flash column chromatography (10% MeOH/CHCl₃ with 1% strong aqueous ammonia) to give compound B10 as a clear syrup (77 mg, 81%). ¹H NMR (400 MHz, CD₃OD) δ 839 (s, 1H), 8.02 (s, 1H), 5.20 (d, J=4.6 Hz, 1H), 5.05 (dd, J=6.6, 4.8 Hz, 1H), 4.76 (dd, J=6.6, 4.4 Hz, 1H), 4.09 (ddd, J=5.8, 4.4, 4.4 Hz, 1H), 2.93 (dd, J=13.4, 4.2 Hz, 1H), 2.87 (dd, J=13.4, 6.0 Hz, 1H), 1.58 (s, 3H), 1.34 (s, 3H). ¹³C NMR (100 MHz, CD₃OD) δ 160.3, 158.7, 155.6, 136.3, 132.4, 117.2, 116.0, 86.6, 86.2, 84.1, 82.4, 44.8, 28.0, 25.9. HRMS (ESI⁺): m/z calcd C₁₄H₁₉N₄O₃S [M+H]⁺ 323.1172, found 323.1161.

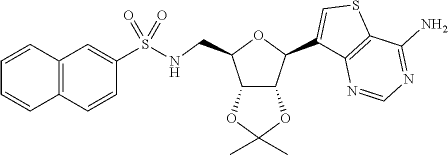 [SMILES: CC1(C)O[C@@H]2[C@H](O1)[C@@H](CNS(=O)(=O)c1ccc3ccccc3c1)O[C@H]2c1csc2c(N)ncnc12]

N-(((3aR,4R,6S,6aS)-6-(4-Aminothieno[3,2-d]pyrimidin-7-yl)-2,2-dimethyltetrahydrofuro[3,4-d][1,3]dioxol-4-yl)methyl)naphthalene-2-sulfonamide (B11). To compound B10 (39 mg, 0.12 mmol) in anhydrous CH₂Cl₂ (2.5 mL) at ° C. were added triethylamine (34 μL, 0.24 mmol), 4-dimethylaminopyridine (3.0 mg, 0.024 mmol), and 2-naphthalenesulfonyl chloride (30 mg, 0.13 mmol). The reaction mixture was allowed to stir at rt for 4 h and diluted with EtOAc (15 mL). The organic layer was washed with water (5×3 mL) and brine (20 mL) and then dried over Na₂SO₄. After filtration, the filtrate was concentrated, and the residue was purified by flash column chromatography (0% to 10% MeOH/CH₂Cl₂) to give compound B11 as a white solid (50 mg, 81%). ¹H NMR (400 MHz, CDCl₃) δ 9.56 (d, J=9.8 Hz, 1H), 9.01 (s, 1H), 8.44 (s, 1H), 7.95-7.75 (m, 4H), 7.69 (s, 1H), 7.63-7.50 (m, 2H), 5.94 (brs, 2H), 5.10 (t, J=6.4 Hz, 1H), 4.94 (d, J=6.6 Hz, 1H), 4.80 (dd, J=6.3, 1.7 Hz, 1H), 4.43 (d, J=1.7 Hz, 1H), 3.56-3.44 (m, 1H), 3.19 (dd, J=12.5, 2.6 Hz, 1H), 1.57 (s, 3H), 1.18 (s, 3H). ¹³C NMR (100 MHz, CDCl₃) δ 158.8, 157.1, 155.2, 137.3, 134.8, 134.3, 132.4, 131.5, 129.7, 129.4, 128.7, 128.2, 128.0, 127.6, 122.4, 116.6, 114.5, 84.0, 83.7, 83.3, 82.0, 45.8, 27.9, 25.4. HRMS (ESI⁺): m/z calcd for C₂₄H₂₅N₄O₅S2 [M+H]⁺ 513.1261, found 513.1263.

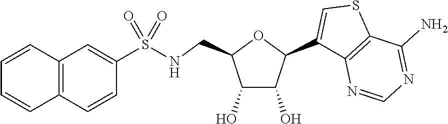 [SMILES: Nc1ncnc2c([C@@H]3O[C@H](CNS(=O)(=O)c4ccc5ccccc5c4)[C@@H](O)[C@H]3O)csc12]

N-(((2R,3S,4R,5S)-5-(4-Aminothieno[3,2-d]pyrimidin-7-yl)-3,4-dihydroxytetrahydrofuran-2-yl)methyl)naphthalene-2-sulfonamide (B12). To compound B11 (40 mg, 0.078 mmol) at 0° C. was added a solution of TFA/H₂O (3 mL, 4:1). The resulting solution was allowed to stir at 0° C. for 30 min, warm to rt, and stir for additional 1 h. The reaction mixture was concentrated, and the residue was co-evaporated with MeOH (6 mL). The residue was then dissolved in MeOH (3 mL) and neutralized with strong aqueous ammonia (three drops). After concentration, the residue was purified by flash column chromatography (0% to 15% MeOH/CH₂Cl₂) to give compound B12 as a white solid (32 mg, 87%). ¹H NMR (400 MHz, CD₃OD) δ 8.62 (s, 1H), 8.43 (d, J=1.5 Hz, 1H), 8.03-7.95 (m, 2H), 7.94-7.88 (m, 2H), 7.82 (dd, J=8.7, 1.9 Hz, 1H), 7.66-7.55 (m, 2H), 4.94 (d, J=8.0 Hz, 1H), 4.61 (dd, J=8.0, 5.5 Hz, 1H), 4.14 (q, J=2.8 Hz, 1H), 4.07 (dd, J=5.5, 2.5 Hz, 1H), 3.34 (dd, J=13.0, 2.9 Hz, 1H), 3.15 (dd, J=12.9, 3.2 Hz, 1H). ¹³C NMR (100 MHz, CD₃OD) δ 160.5, 158.0, 155.5, 138.7, 136.3, 136.0, 133.8, 133.4, 130.8, 130.3, 129.9, 129.2, 129.1, 128.8, 123.4, 117.5, 85.5, 82.2, 75.8, 74.5, 46.7. HRMS (ESI⁺): m/z calcd for C₂₁H₂₁N₄O₅S2 [M+H]⁺ 473.0948, found 473.0946.

### Example 4

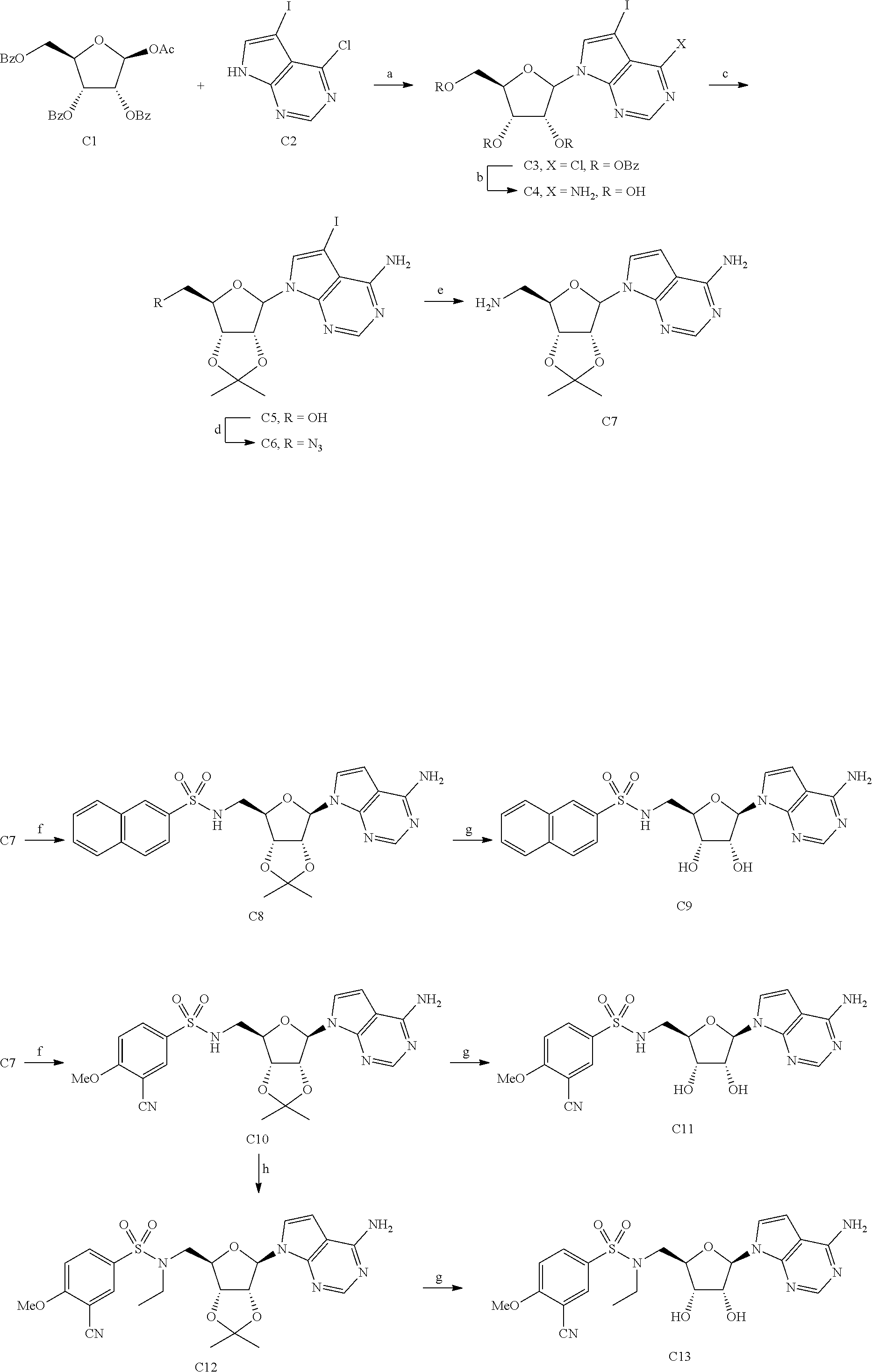

Scheme 7. Reagents and conditions: (a) i. BSA, CH₃CN, rt; ii. TMSOTf, 80° C.; (b) aqueous ammonia, 1,4-dioxane, 60° C.; (c) 2,2-dimethoxypropane, pTsOH, acetone, rt; (d) i. DPPA, DBU, 1,4-dioxane, rt; ii. NaN₃, TBAI, 15-crown-5, reflux; (e) H₂, Pd/C, Et₃N, DMF; (f) arylsulfonyl chloride, Et₃N, DMAP, CH₂Cl₂, rt; (g) TFA/H₂O (4:1), 0° C. to rt; (h) EtOTs, Cs₂CO₃, DMF, 40° C.

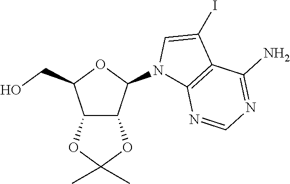 [SMILES: CC1(C)O[C@@H]2[C@H](O1)[C@@H](CO)O[C@H]2n1cc(I)c2c(N)ncnc21]

((3aR,4R,6R,6aR)-6-(4-Amino-5-iodo-7H-pyrrolo[2,3-d]pyrimidin-7-yl)-2,2-dimethyltetrahydrofuro[3,4-d][1,3]dioxol-4-yl)methanol (C₅). To a suspension of known nucleoside C₄ (1.85 g, 4.74 mmol) in acetone (50 mL) were added 2,2-dimethoxypropane (3.00 mL, 24.2 mmol) and pTsOH (993 mg, 5.22 mmol). The resulting mixture was allowed to stir overnight and quenched with saturated NaHCO₃ (25 mL). The solid formed was filtered and washed with acetone. The filtrate was concentrated to remove acetone, and the aqueous layer was extracted with EtOAc (50×3 mL). The combined organic layer was washed with brine and dried over Na₂SO₄. After filtration, the filtrate was concentrated, and the residue was purified by flash column chromatography (0% to 10% MeOH/CH₂Cl₂) to give compound C5 as a white solid (1.48 g, 72%). ¹H NMR (400 MHz, DMSO-d6) δ 8.12 (s, 1H), 7.69 (s, 1H), 6.71 (brs, 2H), 6.18 (d, J=3.4 Hz, 1H), 5.14 (t, J=5.5 Hz, 1H), 5.11 (dd, J=6.4, 3.5 Hz, 1H), 4.89 (dd, J=6.3, 2.9 Hz, 1H), 4.16-4.09 (m, 1H), 3.60-3.47 (m, 2H), 1.53 (s, 3H), 1.30 (s, 3H). ¹³C NMR (100 MHz, DMSO-d6) δ 157.2, 152.1, 149.7, 127.3, 113.1, 103.1, 88.7, 85.4, 83.5, 80.9, 61.5, 52.3, 27.1, 25.2. HRMS (ESI⁺): m/z calcd for C₁₄H₁₈IN₄O₄ [M+H]⁺ 433.0367, found 433. 0375.

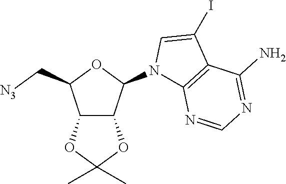 [SMILES: CC1(C)O[C@@H]2[C@H](O1)[C@@H](CN=[N+]=[N-])O[C@H]2n1cc(I)c2c(N)ncnc21]

7-((3aR,4R,6R,6aR)-6-(Azidomethyl)-2,2-dimethyltetrahydrofuro[3,4-d][1,3]dioxol-4-yl)-5-iodo-7H-pyrrolo[2,3-d]pyrimidin-4-amine (C₆). To a solution of compound C5 (708 mg, 1.64 mmol) in anhydrous 1,4-dioxane (10 mL) were added diphenylphosphoryl azide (0.71 mL, 3.28 mmol) and DBU (0.74 mL, 4.91 mmol). After the resulting mixture was allowed to stir at rt for 4 h, sodium azide (533 mg, 8.20 mmol), tetra-n-butylammonium iodide (60 mg, 0.16 mmol) and 15-crown-5 (32 μL, 0.16 mmol) were added, and the reaction mixture was heated at reflux for 4 h. After being allowed to cool to rt, the mixture was diluted with 5% MeOH/EtOAc (30 mL), filtered through a pad of Celite, and washed with 5% MeOH/EtOAc (90 mL). The filtrate was washed with water (30×2 mL) and brine (120 mL). The organic layer was dried over Na₂SO₄ and filtered. The filtrate was concentrated, and the residue was purified by flash column chromatography (0% to 5% MeOH/CH₂Cl₂) to give compound C6 as a yellowish solid (608 mg, 81%). ¹H NMR (400 MHz, DMSO-d6) δ 8.14 (s, 1H), 7.67 (s, 1H), 6.72 (brs, 2H), 6.21 (d, J=3.0 Hz, 1H), 5.29 (dd, J=6.4, 3.0 Hz, 1H), 4.92 (dd, J=6.4, 3.5 Hz, 1H), 4.25-4.15 (m, 1H), 3.64-3.50 (m, 1H), 1.53 (s, 3H), 1.31 (s, 3H). ¹³C NMR (100 MHz, DMSO-d6) δ 157.3, 152.2, 149.7, 127.4, 113.7, 103.2, 88.5, 83.5, 83.1, 81.1, 52.8, 51.6, 26.9, 25.2. HRMS (ESI⁺): m/z calcd for C₁₄H₁₇IN₇O₃ [M+H]⁺ 458.0432, found 458.0440.

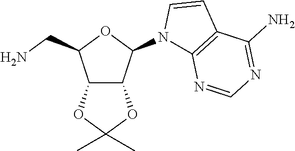 [SMILES: CC1(C)O[C@@H]2[C@H](O1)[C@@H](CN)O[C@H]2n1ccc2c(N)ncnc21]

7-((3aR,4R,6R,6aR)-6-(Aminomethyl)-2,2-dimethyltetrahydrofuro[3,4-d][1,3]dioxol-4-yl)-7H-pyrrolo[2,3-d]pyrimidin-4-amine (C₇). To a solution of compound C6 (312 mg, 0.682 mmol) in DMF (7 mL) was added Et₃N (0.95 mL, 6.82 mmol) and 10% Pd/C (78 mg, 25% w/w), and the mixture was allowed to stir under an atmosphere of hydrogen for 24 h. The mixture was filtered through a pad of Celite and the filtrate was concentrated. The residue was purified by flash column chromatography (10% MeOH/CHCl₃ with 1% strong aqueous ammonia) to give compound C7 as a pale solid (181 mg, 87%). ¹H NMR (400 MHz, CD₃OD) δ 8.10 (s, 1H), 7.25 (d, J=3.7 Hz, 1H), 6.63 (d, J=3.6 Hz, 1H), 6.19 (d, J=3.3 Hz, 1H), 5.28 (dd, J=6.5, 3.3 Hz, 1H), 4.91 (dd, J=6.5, 3.8 Hz, 1H), 4.18-4.08 (m, 1H), 2.94-2.77 (m, 2H), 1.59 (s, 3H), 1.37 (s, 3H). ¹³C NMR (100 MHz, CD₃OD) δ 159.2, 152.7, 151.3, 124.2, 115.9, 105.1, 101.6, 91.2, 87.4, 85.4, 83.2, 44.9, 27.7, 25.8. HRMS (ESI⁺): m/z calcd for C₁₄H₂₀N₅O₃ [M+H]⁺ 306.1561, found 306.1555.

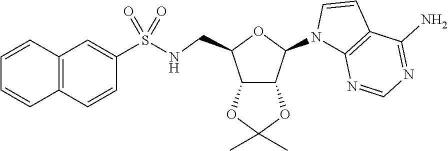 [SMILES: CC1(C)O[C@@H]2[C@H](O1)[C@@H](CNS(=O)(=O)c1ccc3ccccc3c1)O[C@H]2n1ccc2c(N)ncnc21]

N-(((3aR,4R,6R,6aR)-6-(4-Amino-7H-pyrrolo[2,3-d]pyrimidin-7-yl)-2,2-dimethyltetrahydrofuro[3,4-d][1,3]dioxol-4-yl)methyl)naphthalene-2-sulfonamide (C₈). To compound C7 (51 mg, 0.17 mmol) in anhydrous CH₂Cl₂ (3 mL) at ° C. were added triethylamine (47 μL, 0.34 mmol), 4-dimethylaminopyridine (4.3 mg, 0.035 mmol), and 2-naphthalenesulfonyl chloride (45 mg, 0.20 mmol). The reaction mixture was allowed to stir at rt for 3 h and diluted with EtOAc (15 mL). The organic layer was washed with water (5×3 mL) and brine (15 mL) and then dried over Na₂SO₄. After filtration, the filtrate was concentrated, and the residue was purified by flash column chromatography (0% to 10% MeOH/CH₂Cl₂) to give compound C8 as a white solid (65 mg, 78%). ¹H NMR (400 MHz, CDCl₃) δ 9.48 (d, J=9.6 Hz, 1H), 8.65 (s, 1H), 8.42 (d, J=0.9 Hz, 1H), 7.96-7.82 (m, 3H), 7.79 (dd, J=8.7, 1.8 Hz, 1H), 7.64-7.52 (m, 2H), 6.95 (d, J=3.6 Hz, 1H), 6.33 (d, J=3.6 Hz, 1H), 5.64 (d, J=5.0 Hz, 1H), 5.52 (brs, 2H), 5.34 (t, J=5.7 Hz, 1H), 4.92 (dd, J=6.3, 1.9 Hz, 1H), 4.44 (d, J=2.0 Hz, 1H), 3.56-3.45 (m, 1H), 3.19 (dd, J=12.8, 2.4 Hz, 1H), 1.57 (s, 3H), 1.22 (s, 3H). ¹³C NMR (100 MHz, CDCl₃) δ 157.6, 152.4, 149.0, 137.3, 134.9, 132.4, 129.7, 129.4, 128.8, 128.3, 128.0, 127.6, 125.6, 122.4, 114.6, 105.4, 98.3, 95.7, 82.5 (×2), 81.8, 45.5, 27.7, 25.3. HRMS (ESI⁺): m/z calcd for C₂₄H₂₆N₅O₅S [M+H]⁺ 496.1649, found 496.1652.

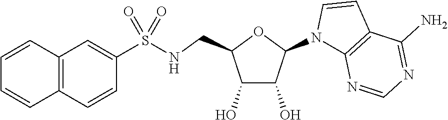 [SMILES: Nc1ncnc2c1ccn2[C@@H]1O[C@H](CNS(=O)(=O)c2ccc3ccccc3c2)[C@@H](O)[C@H]1O]

N-(((2R,3S,4R,5R)-5-(4-Amino-7H-pyrrolo[2,3-d]pyrimidin-7-yl)-3,4-dihydroxytetrahydrofuran-2-yl)methyl)naphthalene-2-sulfonamide (C₉). To compound C8 (51 mg, 0.10 mmol) at 0° C. was added a solution of TFA/H₂O (3 mL, 4:1). The resulting solution was allowed to stir at 0° C. for 30 min, warm to rt, and stir for additional 1 h. The reaction mixture was concentrated, and the residue was co-evaporated with MeOH (5 mL). The residue was then dissolved in MeOH (3 mL) and neutralized with strong aqueous ammonia (two drops). After concentration, the residue was purified by flash column chromatography (0% to 15% MeOH/CH₂Cl₂) to give compound C9 as a white solid (37 mg, 79%). ¹H NMR (400 MHz, CD₃OD) δ 8.41 (s, 1H), 8.24 (s, 1H), 8.04-7.85 (m, 3H), 7.80 (d, J=8.5 Hz, 1H), 7.65-7.51 (m, 2H), 7.16 (d, J=3.3 Hz, 1H), 6.56 (d, J=3.3 Hz, 1H), 5.80 (d, J=6.6 Hz, 1H), 4.85-4.75 (m, 1H, partially buried under solvent peaks), 4.24-4.06 (m, 2H), 3.40-3.25 (m, 1H), 3.18 (dd, J=13.2, 2.6 Hz, 1H). ¹³C NMR (100 MHz, CD₃OD) δ 159.3, 152.3, 150.5, 138.6, 136.3, 133.7, 130.7, 130.3, 129.9, 129.2, 129.1, 128.8, 125.8, 123.4, 106.0, 100.6, 92.5, 85.1, 74.3, 73.1, 46.4. HRMS (ESI⁺): m/z calcd for C₂₁H₂₂N₅O₅S [M+H]⁺ 456.1336, found 456.1333.

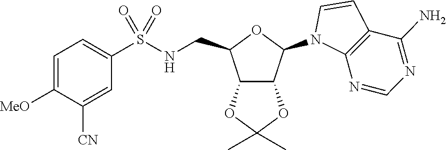 [SMILES: COc1ccc(S(=O)(=O)NC[C@H]2O[C@@H](n3ccc4c(N)ncnc43)[C@@H]3OC(C)(C)O[C@@H]32)cc1C#N]

N-(((3aR,4R,6R,6aR)-6-(4-Amino-7H-pyrrolo[2,3-d]pyrimidin-7-yl)-2,2-dimethyltetrahydrofuro[3,4-d][1,3]dioxol-4-yl)methyl)-3-cyano-4-methoxybenzenesulfonamide (C10). Amine C₇ (108 mg, 0.354 mmol) was treated with 3-cyano-4-methoxybenzenesulfonyl chloride (98 mg, 0.423 mmol) to give compound C10 as a white solid (136, 77%). ¹H NMR (400 MHz, CDCl₃) δ 9.44 (d, J=9.1 Hz, 1H), 8.53 (s, 1H), 7.98-7.90 (m, 2H), 7.04-6.93 (m, 2H), 6.36 (d, J=3.6 Hz, 1H), 5.67 (d, J=4.8 Hz, 1H), 5.55 (brs, 2H), 5.33 (dd, J=6.3, 4.8 Hz, 1H), 5.04 (dd, J=6.4, 2.2 Hz, 1H), 4.46 (d, J=2.2 Hz, 1H), 3.96 (s, 3H), 3.53-3.42 (m, 1H), 3.13 (dd, J=12.5, 2.5 Hz, 1H), 1.60 (s, 3H), 1.34 (s, 3H). ¹³C NMR (100 MHz, CDCl₃) δ 163.8, 157.7, 152.2, 148.9, 133.4, 133.2, 133.1, 125.6, 115.0, 114.8, 112.0, 105.4, 102.8, 98.5, 95.5, 82.7, 82.5, 81.6, 56.9, 45.5, 27.6, 25.4. HRMS (ESI⁺): m/z calcd for C₂₂H₂₅N₆O₆S [M+H]⁺ 501.1551, found 501.1548.

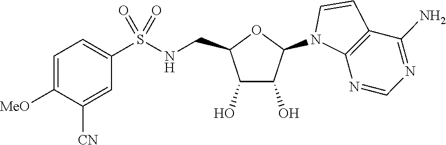 [SMILES: COc1ccc(S(=O)(=O)NC[C@H]2O[C@@H](n3ccc4c(N)ncnc43)[C@H](O)[C@@H]2O)cc1C#N]

N-(((2R,3S,4R,5R)-5-(4-Amino-7H-pyrrolo[2,3-d]pyrimidin-7-yl)-3,4-dihydroxytetrahydrofuran-2-yl)methyl)-3-cyano-4-methoxybenzenesulfonamide (C11). Compound C10 was deprotected to give compound Cl₁ as a pale solid (15 mg, 71%). ¹H NMR (400 MHz, CD₃OD) δ 8.19 (s, 1H), 8.07-7.97 (m, 2H), 7.23 (d, J=8.9 Hz, 1H), 7.17 (d, J=3.7 Hz, 1H), 6.60 (d, J=3.6 Hz, 1H), 5.83 (d, J=6.3 Hz, 1H), 4.70 (t, J=6.0 Hz, 1H), 4.18 (dd, J=5.6, 3.3 Hz, 1H), 4.10 (q, J=3.5 Hz, 1H), 3.98 (s, 3H), 3.32-3.25 (m, 1H, partially buried under solvent peaks), 3.20 (dd, J=13.4, 3.3 Hz, 1H). ¹³C NMR (100 MHz, CD₃OD) δ 165.3, 159.1, 152.1, 150.5, 134.8, 134.6, 134.0, 125.6, 116.0, 113.6, 105.8, 103.2, 100.8, 92.2, 84.7, 74.5, 73.0, 57.6, 46.3. HRMS (ESI⁺): m/z calcd for C₁₉H₂₁N₆O₆S [M+H]⁺ 461.1238, found 461.1243.

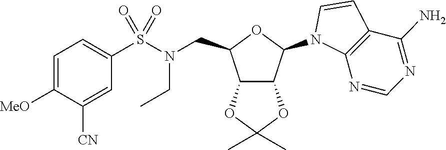 [SMILES: CCN(C[C@H]1O[C@@H](n2ccc3c(N)ncnc32)[C@@H]2OC(C)(C)O[C@@H]21)S(=O)(=O)c1ccc(OC)c(C#N)c1]

N-(((3aR,4R,6R,6aR)-6-(4-Amino-7H-pyrrolo[2,3-d]pyrimidin-7-yl)-2,2-dimethyltetrahydrofuro[3,4-d][1,3]dioxol-4-yl)methyl)-3-cyano-N-ethyl-4-methoxybenzenesulfonamide (C12). To a solution of compound C10 (48 mg, 0.096 mmol) were added Cs₂CO₃ (47 mg, 0.14 mmol) and EtOTs (29 mg, 0.14 mmol), and the resulting mixture was heated at 40° C. for 16 h. Since TLC showed that the reaction was not complete, additional Cs₂CO₃ (31 mg, 0.095 mmol) and EtOTs (20 mg, 0.10 mmol) were added, and the mixture was heated at 40° C. for 18 h. Subsequentially, additional Cs₂CO₃ (16 mg, 0.049 mmol) and EtOTs (10 mg, 0.050 mmol) were added, and the mixture was heated at 40° C. for another 18 h. The reaction mixture was diluted with EtOAc (15 mL), and the organic layer was washed with water (5×2 mL) and brine (15 mL) and then dried over Na₂SO₄. After filtration, the filtrate was concentrated, and the residue was purified by flash column chromatography (0% to 10% MeOH/CH₂Cl₂) to give compound C12 as a pale solid (26 mg, 51%). ¹H NMR (400 MHz, CDCl₃) δ 8.30 (s, 1H), 7.91 (d, J=2.3 Hz, 1H), 7.83 (dd, J=8.9, 2.3 Hz, 1H), 7.04 (d, J=3.6 Hz, 1H), 6.88 (d, J=9.0 Hz, 1H), 6.45 (d, J=3.6 Hz, 1H), 6.11 (d, J=1.8 Hz, 1H), 5.44 (brs, 2H), 5.40 (dd, J=6.4, 1.8 Hz, 1H), 5.14 (dd, J=6.4, 3.4 Hz, 1H), 4.37-4.27 (m, 1H), 3.95 (s, 3H), 3.56 (dd, J=14.7, 6.0 Hz, 1H), 3.37-3.20 (m, 2H), 3.18-3.06 (m, 1H), 1.59 (s, 3H), 1.39 (s, 3H), 1.01 (t, J=7.1 Hz, 3H). ¹³C NMR (100 MHz, CDCl₃) δ 163.7, 157.1, 152.3, 150.2, 133.7, 133.3, 132.9, 124.1, 115.1, 114.5, 111.6, 104.2, 102.6, 99.3, 91.6, 85.1, 84.4, 82.7, 56.8, 48.9, 43.6, 27.3, 25.5, 13.7. HRMS (ESI⁺): m/z calcd for C₂₄H₂₉N₆O₆S [M+H]⁺ 529.1864, found 529.1875.

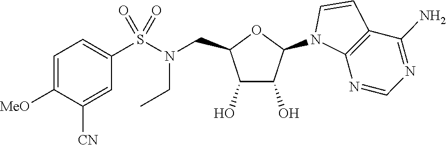 [SMILES: CCN(C[C@H]1O[C@@H](n2ccc3c(N)ncnc32)[C@H](O)[C@@H]1O)S(=O)(=O)c1ccc(OC)c(C#N)c1]

N-(((2R,3S,4R,5R)-5-(4-Amino-7H-pyrrolo[2,3-d]pyrimidin-7-yl)-3,4-dihydroxytetrahydrofuran-2-yl)methyl)-3-cyano-N-ethyl-4-methoxybenzenesulfonamide (C₁₃). Compound C₁₂ (24 mg, 0.045 mmol) was deprotected to give compound C13 as a pale solid (20 mg, 90%). ¹H NMR (400 MHz, C_{D}-OD) δ 8.14 (s, 1H), 8.05-7.94 (m, 2H), 7.32 (d, J=3.8 Hz, 1H), 7.12 (d, J=9.0 Hz, 1H), 6.73 (d, J=3.7 Hz, 1H), 6.06 (d, J=4.8 Hz, 1H), 4.50 (t, J=5.2 Hz, 1H), 4.22 (t, J=5.4 Hz, 1H), 4.15-4.06 (m, 1H), 3.95 (s, 3H), 3.66 (dd, J=15.0, 3.6 Hz, 1H), 3.55 (dd, J=15.0, 8.4 Hz, 1H), 3.47-3.35 (m, 1H), 3.28-3.18 (m, 1H), 1.11 (t, J=7.1 Hz, 3H). ¹³C NMR (100 MHz, CD₃OD) δ 165.3, 156.8, 150.6, 149.0, 135.3, 134.5, 134.4, 124.9, 115.9, 113.3, 104.7, 103.1, 102.4, 90.3, 82.9, 75.2, 73.2, 57.6, 50.6, 44.3, 14.2. HRMS (ESI⁺): m/z calcd for C₂₁H₂₅N₆O₆S [M+H]⁺ 489.1551, found 489.1547.

### TABLES

TABLE 1
Bi-substrate Inhibitors of SARS-CoV-2 Nsp14 MTase

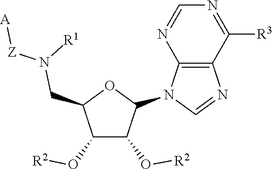

1-9

| | | | | | MTase | | |
|---|---|---|---|---|---|---|---|
| | | | | | IC₅₀ | EC₅₀ | CC₅₀ |
| Cmpd | A—Z* | R¹ | R¹ | R³ | (μM) | (μM) | (μM) |
| 1 | 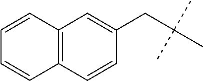 | H | H | NH₂ | 9.6 | NA^{a} | NT^{b} |
| 2 | 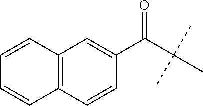 | H | H | NH₂ | 2.7 | NA^{a} | NT^{b} |
| 3 | 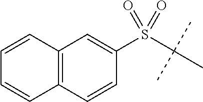 | H | H | NH₂ | 0.061 | NA^{a} | NT^{b} |
| 4 | 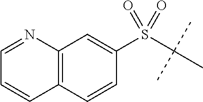 | H | H | NH₂ | 0.069 | NA^{a} | NT^{b} |
| 5 | 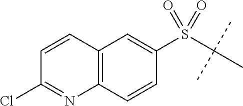 | H | H | NH₂ | 0.44 | NA^{a} | NT^{b} |
| 6 | 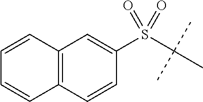 | H | H | NHC(O)CH₃ | 0.048 | NA^{a} | >100 |
| 7 | 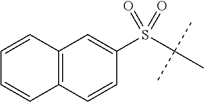 | H | 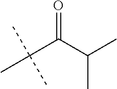 | NH₂ | — | ca. 97 | >100 |
| 8 | 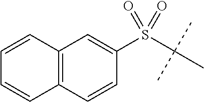 | H | Ac | NH₂ | — | 53 | >100 |
| 9 | 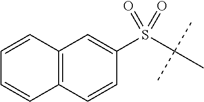 | H | Ac | NHC(O)CH₃ | — | 61 | >100 |

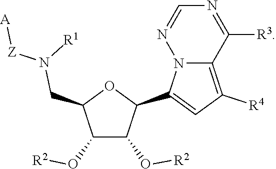

10-14, 39, 41, 43, 45, 47, 49, 51 and 53

| | | | | | | MTase | | |
|---|---|---|---|---|---|---|---|---|
| | | | | | | IC₅₀ | EC₅₀ | CC₅₀ |
| Cmpd | A—Z* | R¹ | R² | R³ | R⁴ | (μM) | (μM) | (μM) |
| 10 | 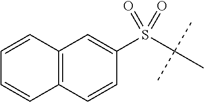 | H | H | NH₂ | H | 0.093 | 0.72 | >100 |
| 11 | 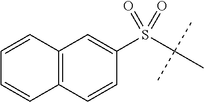 | H | Ac | NH₂ | H | — | 4.3 | 63 |
| 12 | 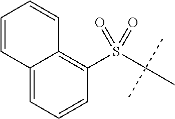 | H | H | NH₂ | H | 7.3 | 3.0 | >100 |
| 13 | 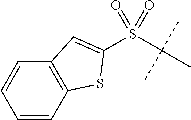 | H | H | NH₂ | H | 0.042 | 1.6 | >100 |
| 14 | 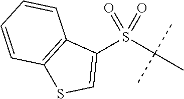 | H | H | NH₂ | H | 2.0 | 13 | >100 |
| 39 | 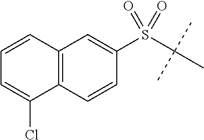 | H | H | NH₂ | H | 0.049 μM^{c} | | |
| 41 | 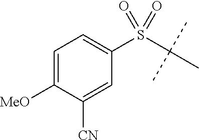 | H | H | NH₂ | H | 94% inhibition at 10 μM^{c} | | |
| 43 | 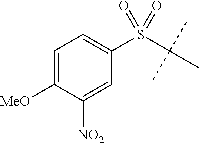 | H | H | NH₂ | H | 79% inhibition at 10 μM^{c} | | |
| 45 | 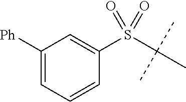 | H | H | NH₂ | H | 85% inhibition at 10 μM^{c} | | |
| 47 | 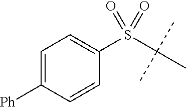 | H | H | NH₂ | H | 85% inhibition at 10 μM^{c} | | |
| 49 | 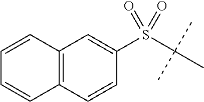 | H | H | NH₂ | I | 0.046 | 11 | 28 |
| 51 | 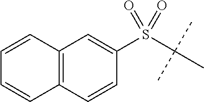 | H | H | NH₂ | F | 0.032 μM^{c} | | |
| 53 | 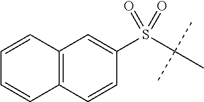 | Et | H | NH₂ | H | 0.156 μM^{c} | | |

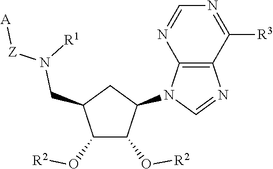

A1-A2

| | | | | | MTase | | |
|---|---|---|---|---|---|---|---|
| | | | | | IC₅₀ | EC₅₀ | CC₅₀ |
| Cmpd | A—Z* | R¹ | R² | R³ | (μM) | (μM) | (μM) |
| A1 | 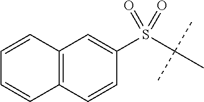 | H | H | NH₂ | 0.78 | 33% inhibition at 10 μM | >100 |
| A2 | 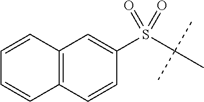 | Et | H | NH₂ | 0.26 | 29% inhibition at 10 μM | >100 |

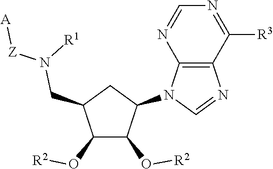

A3-A4

| | | | | | MTase | | |
|---|---|---|---|---|---|---|---|
| | | | | | IC₅₀ | EC₅₀ | CC₅₀ |
| Cmpd | A—Z* | R¹ | R² | R³ | (μM) | (μM) | (μM) |
| A3 | 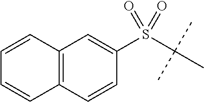 | H | H | NH₂ | 103 | 41% inhibition at 10 μM | >100 |
| A4 | 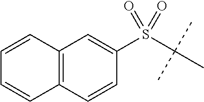 | Et | H | NH₂ | 5.1 | 34% inhibition at 10 μM | >100 |

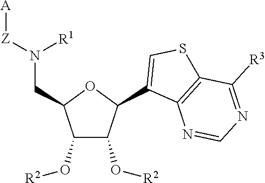

B12

| | | | | | MTase | | |
|---|---|---|---|---|---|---|---|
| | | | | | IC₅₀ | EC₅₀ | CC₅₀ |
| Cmpd | A—Z* | R¹ | R² | R³ | (μM) | (μM) | (μM) |
| B12 | 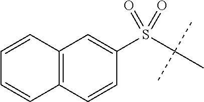 | H | H | NH₂ | 91% inhibition at 10 μM^{c} | | |

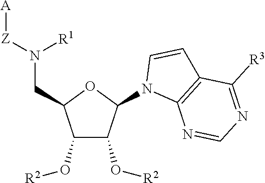

C9, C11, and C13

| | | | | | MTase | | |
|---|---|---|---|---|---|---|---|
| | | | | | IC₅₀ | EC₅₀ | CC₅₀ |
| Cmpd | A—Z* | R¹ | R² | R³ | (μM) | (μM) | (μM) |
| C9 | 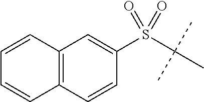 | H | H | NH₂ | 0.019^{c} | | |
| C11 | 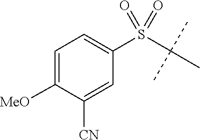 | H | H | NH₂ | 98% inhibition at 10 μM^{c} | | |
| C13 | 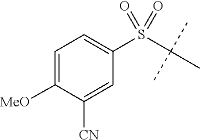 | Et | H | NH₂ | 100% inhibition at 10 μM^{c} | | |

Comparative Example

| | | | | | |
|---|---|---|---|---|---|
| sinefungin | — | — | 0.26 | NA^{a} | NT^{b} |
| remdesivir | — | — | — | 0.093 | 43 |

^{a}NA, not active, <15% inhibition at 50 μM.
^{b}NT, nontoxic, >95% cell viability at 50 μM;
^{c}Methylation reaction detected by MTase-Glo ™ methyltransferase assay kit.

TABLE 2
In vitro metabolic stabilities and
permeability of selected compounds

| | Plasma Stability | | Human Liver S9 Stability | |
|---|---|---|---|---|
| | t_{1/2} (h), n = 3 | | t_{1/2} (min), n = 2 | |
| Cmpd | Human | Mouse | Phase I^{b} | Phase II^{c} |
| 3 | >24^{d} | >24^{d} | 674 ± 136 | >45^{e} |
| 10 | >24^{d} | >24^{d} | 842 ± 210 | >45^{e} |
| Verapamil | | | 19.2 ± 0.8 | |
| Umbelliferone | | | | 3.0 ± 0.05 |

| | Mouse Liver S9 Stability | | |
|---|---|---|---|
| | t_{1/2} (min), n = 2 | | PAMPA Permeability |
| Cmpd | Phase I^{b} | Phase II^{c} | Pe^{a} (10⁻⁶ cm/s), n = 6 |
| 3 | 273 ± 23 | >45^{e} | 0.2 ± 0.1 |
| 10 | 28.9 ± 0.3 | >45^{e} | 0.2 ± 0.1 |
| Verapamil | 5.7 ± 0.1 | | |
| Umbelliferone | | 0.8 ± 0.005 | |

Data are presented as mean±SD. ^{a}Pe, Apparent permeability coefficient. ^{b}CYP enzyme cofactor: NADPH. ^{c}Cofactor: UDPGA. ^{d}No decrease in remaining percentage was observed at the end of incubation (24 h). ^{e}No decrease in remaining percentage was observed at the end of incubation (45 min).

TABLE 3
Screening of compound 3 against a panel of 10 human MTases.

| Human | % Inhibition | Reference |
|---|---|---|
| MTase | at 10 μM | compound |
| G9a | 11 | UNC0646 |
| DOT1L | 10 | SGC0946 |
| MLL1 Complex | 1 | SAH |
| PRMT1 | 5 | MS023 |
| PRMT3 | 0 | SGC707 |
| SUV39H1 | 11 | Chaetocin |
| METTL3/METTL14 | 10 | SAH |
| DNMT1 | 5 | SAH |
| DNMT3A/DNMT3L | 4 | SAH |
| EZH1 | 2 | GSK343 |

1. A compound of formula 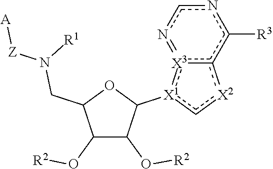
or a pharmaceutically acceptable salt or prodrug thereof,
wherein—
(i) X¹ is C, X² is C—R⁴, and X³ is N, or (ii) X¹ is N, X² is N, and X³ is C, or (iii) X¹ is C, X² is S, X³ is C, or (iv) X¹ is N, X² is C—R⁴, and X³ is C;
R¹ is H or an alkyl;
each R² is independently selected from H, an alkyl, and a —C(O)-alkyl;
R³ is an amino or an amido;
R⁴ is H or a halo;
Q is O or —CH₂—.
Z is a sulfonyl, a carbonyl, or an alkylene; and
A is an aryl, a heteroaryl, or a biaryl, wherein the aryl or heteroaryl is optionally substituted in one or more positions with a halo, an alkyl optionally substituted at one or more position with a halo, an alkoxyl optionally substituted at one or more position with a halo, nitro, or cyano; and
wherein when X¹ is N, X² is N, and X³ is C; R² is H; R³ is amino; and Q is O, then A is not a monocyclic aryl.

2. The compound of claim 1, wherein X¹ is C, X² is C—R⁴, and X³ is N.

3. The compound or claim 2, wherein the compound has a formula of

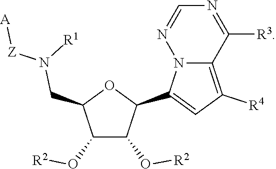

4. The compound of claim 1, wherein X¹ is N, X² is N, and X³ is C.

5. The compound of claim 4, the compound has a formula of

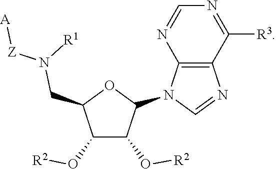

6. The compound of claim 4, wherein the compound has a formula of

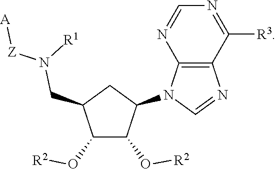

7. The compound of claim 4, wherein the compound has a formula of

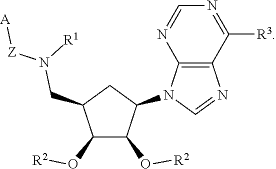

8. The compound of claim 1, wherein X¹ is C, X² is S, and X³ is C.

9. The compound of claim 8, wherein the compound has a formula of

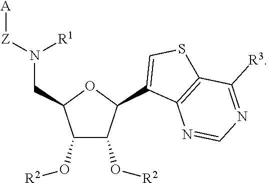

10. The compound of claim 1, wherein X¹ is N, X² is C—R⁴, and X³ is C.

11. The compound of claim 10, wherein the compound has a formula of

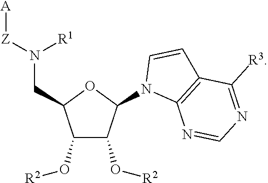

12. The compound of claim 1,
wherein A is a naphthyl, a quinolinyl, or a benzothiophenyl, wherein the naphthyl, quinolinyl, or the benzothiophenyl is optionally substituted in one or more positions with a halo; or
wherein A is phenyl optionally substituted with an alkoxyl, nitro, cyano, or any combination thereof; or
wherein A is biphenyl.

13. The compound of claim 12, wherein A is naphthyl, halo-substituted naphthyl, or benzothiophenyl.

14. The compound of claim 1, wherein Z is —S(O)₂—, —C(O)—, or —CH₂—.

15. The compound of claim 1, wherein R³ is —NH₂ or —NHC(O)CH₃.

16. The compound of claim 1, wherein R² is H, —C(O)CH₃, or —C(O)CH(CH₃)₂.

17. The compound of claim 1, wherein R¹ is H or ethyl.

18. The compound of claim 1, wherein the compound is selected from:
(2R,3R,4S,5R)-2-(6-Amino-9H-purin-9-yl)-5-(((naphthalen-2-ylmethyl)amino)methyl)tetrahydrofuran-3,4-diol (1);
N-(((2R,3S,4R,5R)-5-(6-Amino-9H-purin-9-yl)-3,4-dihydroxytetrahydrofuran-2-yl)methyl)-2-naphthamide (2);
N-(((2R,3S,4R,5R)-5-(6-Amino-9H-purin-9-yl)-3,4-dihydroxytetrahydrofuran-2-yl)methyl)naphthalene-2-sulfonamide (3);
N-(((2R,3 S,4R,5R)-5-(6-Amino-9H-purin-9-yl)-3,4-dihydroxytetrahydrofuran-2-yl)methyl)quinoline-7-sulfonamide (4);
N-(((2R,3 S,4R,5R)-5-(6-Amino-9H-purin-9-yl)-3,4-dihydroxytetrahydrofuran-2-yl)methyl)-2-chloroquinoline-6-sulfonamide (5);
N-(9-((2R,3R,4S,5R)-3,4-Dihydroxy-5-((naphthalene-2-sulfonamido)methyl)tetrahydrofuran-2-yl)-9H-purin-6-yl)acetamide (6);
(2R,3R,4R,5R)-2-(6-Amino-9H-purin-9-yl)-5-((naphthalene-2-sulfonamido)methyl)tetrahydrofuran-3,4-diyl bis(2-methylpropanoate) (7)
(2R,3R,4R,5R)-2-(6-Amino-9H-purin-9-yl)-5-((naphthalene-2-sulfonamido)methyl)tetrahydrofuran-3,4-diyl diacetate (8);
(2R,3R,4R,5R)-2-(6-Acetamido-9H-purin-9-yl)-5-((naphthalene-2-sulfonamido)methyl)tetrahydrofuran-3,4-diyl diacetate (9);
N-(((2R,3S,4R,5S)-5-(4-Aminopyrrolo[2,1-f][1,2,4]triazin-7-yl)-3,4-dihydroxytetrahydrofuran-2-yl)methyl)naphthalene-2-sulfonamide (10);
(2S,3S,4R,5R)-2-(4-Aminopyrrolo[2,1-f][1,2,4]triazin-7-yl)-5-((naphthalene-2-sulfonamido)methyl)tetrahydrofuran-3,4-diyl diacetate (11);
N-(((2R,3S,4R,5S)-5-(4-Aminopyrrolo[2,1-f][1,2,4]triazin-7-yl)-3,4-dihydroxytetrahydrofuran-2-yl)methyl)naphthalene-1-sulfonamide (12);
N-(((2R,3S,4R,5S)-5-(4-aminopyrrolo[2,1-f][1,2,4]triazin-7-yl)-3,4-dihydroxytetrahydrofuran-2-yl)methyl)benzo[b]thiophene-2-sulfonamide (13);
N-(((2R,3S,4R,5S)-5-(4-Aminopyrrolo[2,1-f][1,2,4]triazin-7-yl)-3,4-dihydroxytetrahydrofuran-2-yl)methyl)benzo[b]thiophene-3-sulfonamide (14);
N-(((1R,2R,3 S,4R)-4-(6-Amino-9H-purin-9-yl)-2,3-dihydroxycyclopentyl)methyl)naphthalene-2-sulfonamide (A1);
N-(((1R,2R,3 S,4R)-4-(6-Amino-9H-purin-9-yl)-2,3-dihydroxycyclopentyl)methyl)-N-ethylnaphthalene-2-sulfonamide (A2);
N-(((1R,2S,3R,4R)-4-(6-Amino-9H-purin-9-yl)-2,3-dihydroxycyclopentyl)methyl)naphthalene-2-sulfonamide (A3);
N-(((1R,2S,3R,4R)-4-(6-Amino-9H-purin-9-yl)-2,3-dihydroxycyclopentyl)methyl)-N-ethylnaphthalene-2-sulfonamide (A4);
N-(((2R,3S,4R,5S)-5-(4-aminopyrrolo[2,1-f][1,2,4]triazin-7-yl)-3,4-dihydroxytetrahydrofuran-2-yl)methyl)-5-chloronaphthalene-2-sulfonamide (39);
N-(((2R,3S,4R,5S)-5-(4-aminopyrrolo[2,1-f][1,2,4]triazin-7-yl)-3,4-dihydroxytetrahydrofuran-2-yl)methyl)-3-cyano-4-methoxybenzenesulfonamide (41);
N-(((2R,3S,4R,5S)-5-(4-aminopyrrolo[2,1-f][1,2,4]triazin-7-yl)-3,4-dihydroxytetrahydrofuran-2-yl)methyl)-4-methoxy-3-nitrobenzenesulfonamide (43);
N-(((2R,3S,4R,5S)-5-(4-Aminopyrrolo[2,1-f][1,2,4]triazin-7-yl)-3,4-dihydroxytetrahydrofuran-2-yl)methyl)-[1,1′-biphenyl]-3-sulfonamide (45);
N-(((3aR,4R,6S,6aS)-6-(4-Aminopyrrolo[2,1-f][1,2,4]triazin-7-yl)-2,2-dimethyltetrahydrofuro[3,4-d][1,3]dioxol-4-yl)methyl)-[1,1′-biphenyl]-4-sulfonamide (47);
N-(((2R,3S,4R,5S)-5-(4-Amino-5-iodopyrrolo[2,1-f][1,2,4]triazin-7-yl)-3,4-dihydroxytetrahydrofuran-2-yl)methyl)naphthalene-2-sulfonamide (49);
N-(((2R,3S,4R,5S)-5-(4-Amino-5-fluoropyrrolo[2,1-f][1,2,4]triazin-7-yl)-3,4-dihydroxytetrahydrofuran-2-yl)methyl)naphthalene-2-sulfonamide (51);
N-(((2R,3S,4R,5S)-5-(4-Aminopyrrolo[2,1-f][1,2,4]triazin-7-yl)-3,4-dihydroxytetrahydrofuran-2-yl)methyl)-N-ethylnaphthalene-2-sulfonamide (53);
N-(((2R,3S,4R,5S)-5-(4-Aminothieno[3,2-d]pyrimidin-7-yl)-3,4-dihydroxytetrahydrofuran-2-yl)methyl)naphthalene-2-sulfonamide (B12);
N-(((2R,3S,4R,5R)-5-(4-Amino-7H-pyrrolo[2,3-d]pyrimidin-7-yl)-3,4-dihydroxytetrahydrofuran-2-yl)methyl)naphthalene-2-sulfonamide (C9);
N-(((2R,3S,4R,5R)-5-(4-Amino-7H-pyrrolo[2,3-d]pyrimidin-7-yl)-3,4-dihydroxytetrahydrofuran-2-yl)methyl)-3-cyano-4-methoxybenzenesulfonamide (C11); and
N-(((2R,3S,4R,5R)-5-(4-Amino-7H-pyrrolo[2,3-d]pyrimidin-7-yl)-3,4-dihydroxytetrahydrofuran-2-yl)methyl)-3-cyano-N-ethyl-4-methoxybenzenesulfonamide (C13);
or a pharmaceutically acceptable salt thereof.

19-20. (canceled)

21. A pharmaceutical composition comprising the compound according to claim 1 and a pharmaceutically acceptable excipient, carrier, or diluent.

22-24. (canceled)

25. A method for treating a subject in need of a treatment for a viral infection, the method comprising administering an effective among of the compound according to claim 1 to treat the viral infection.

26-27. (canceled)

28. A method of inhibiting viral replication, the method comprising contacting a cellular medium having a virus therein with an effective amount of the compound according to claim 1 to inhibit replication of the virus.

29-32. (canceled)
